(19) United States
(12) Patent Application Publication
(10) Pub. No.: US 2026/0201476 A1
(43) Pub. Date: Jul. 16, 2026

(54) METHODS FOR IDENTIFYING RESISTANCE OR RESPONSE TO VENETOCLAX/HYPOMETHYLATING AGENT/ANTI-CD70 AGENT TREATMENT FOR ACUTE MYELOID LEUKEMIA

(71) Applicants: Clayton Smith, Denver, CO (US); Mohammad Islam, Victoria, MN (US); Justin Dale, Edina, MN (US); Craig T. Jordan, Aurora, CA (US); James Coates, North Ridgeville, OH (US); Nazmul Islam, Denver, CO (US); Maura Gasparetto, Denver, CO (US); Jamie Reuben, Denver, CO (US); Karan Sapiah, Denver, CO (US); The Regents of the University of Colorado, A Body Corporate, Denver, CO (US)

(72) Inventors: Clayton Smith, Denver, CO (US); Md Nazmul Islam, Victoria, MN (US); Justin Dale, Edina, MN (US); Craig T. Jordan, Aurora, CO (US); James Coates, North Ridgeville, OH (US); Maura Gasparetto, Denver, CO (US); Jamie Reuben, Denver, CO (US); Karan Sapiah, Denver, CO (US)

(73) Assignee: The Regents of the University of Colorado, A Body Corporate, Denver, CO (US)

(21) Appl. No.: 19/136,706

(22) PCT Filed: Dec. 8, 2023
(86) PCT No.: PCT/US2023/083087

§ 371 Date: Jun. 6, 2025

Related U.S. Application Data
(60) Provisional application No. 63/386,506, filed on Dec. 8, 2022.
(60) Provisional application No. 63/490,276, filed on Mar. 15, 2023.

(51) Int. Cl. C12Q 1/6886; C12Q 1/6827

(52) U.S. Cl. CPC C12Q 1/6886; C12Q 1/6827; C12Q 2600/106; C12Q 2600/156; C12Q 2600/158

(57) ABSTRACT

The present disclosure provides methods of treating acute myeloid leukemia (AML) and methods of determining responsiveness to AML treatment regimens, including regimens comprising the administration of a BCL-2 inhibitor, a hypomethylating agent, a CD70-targeting agent, or any combination thereof, the methods comprising identifying the presence or absence of one or more biomarkers described herein.

### RELATED APPLICATIONS

This application claims priority to, and the benefit of, U.S. Provisional Application No. 63/386,506 filed on Dec. 8, 2022, and U.S. Provisional Application No. 63/490,276 filed on Mar. 15, 2023. The contents of each of the aforementioned patent applications are incorporated herein by reference in their entireties.

### BACKGROUND

Acute myeloid leukemia (AML) is a blood cancer in which the bone marrow of a subject makes abnormal myeloblasts, red blood cells, or platelets. AML is one of the most common forms of acute leukemia in adults. The build-up of AML cells in bone marrow and blood can rapidly lead to infection, anemia, excessive bleeding and death. BCL-2 inhibitor venetoclax has recently emerged as an important component of therapy for acute myeloid leukemia (AML). In combination with a number of backbone chemotherapy treatments, venetoclax can induce responses in approximately 60-70% of older previously untreated AML patients, many of whom are unfit for conventional induction therapy. Accordingly, treatment with venetoclax in combination with a hypomethylating agent (HMA), such as azacitidine or decitabine has emerged as the standard of care for AML, particularly within elderly patients.

However, resistance to these venetoclax-based therapies has been documented, as well as relapse following initial response. Thus, additional therapies have recently been developed for treating AML, including antibodies, cellular immunotherapies and other agents directed at CD70 or CD70 biologic processes. Accordingly, there is a need a need in the art for methods of predicting a patient's response to particular treatments, including treatment with a combination venetoclax and an HMA, optionally in combination with a CD70-targeting agent. There is also a need in the art for methods of treating AML in subjects who exhibit specific cellular biomarkers.

### SUMMARY

The present disclosure provides methods of identifying that a subject having AML will be responsive to an AML treatment comprising the administration of a combination of at least one BCL-2 inhibitor and at least one hypomethylating agent, the methods comprising: a) determining the mutational status of at least one of BCORL1, EZH2, IDH1, IDH2, NPM1, RAD21, SF3B1, SH2B3 and KIT in a plurality of cells in a sample from the subject; b) identifying that the subject will be responsive to the AML treatment when the presence of at least one mutation in at least one BCORL1, EZH2, IDH1, IDH2, NPM1, RAD21, SF3B1, SH2B3 and KIT is identified.

The present disclosure provides methods of identifying if a subject having AML will be responsive to an AML treatment comprising the administration of a combination of at least one BCL-2 inhibitor and at least one hypomethylating agent, the methods comprising: a) determining the cytogenetic status of a plurality of cells in a sample from the subject; and b) identifying that the subject will be responsive to the AML treatment when the presence of at least cell exhibiting at least one of: i) a Del5q phenotype; ii) a CBFB Loss phenotype; and iii) a Normal Cytogenetics phenotype is identified.

The present disclosure provides methods of identifying that a subject having AML will be responsive to an AML treatment comprising the administration of a combination of at least one BCL-2 inhibitor and at least one hypomethylating agent, the methods comprising: a) measuring the expression of at least CD10 in a plurality of cells in a sample from the subject; b) identifying the presence of at least one CD10+ cell based on the expression measured in step (a); c) identifying that the subject will be responsive to the AML treatment when the presence of at CD10+ cell is identified.

In some aspects, the methods further comprise administering to the subject identified as being responsive to the AML treatment at least one amount of at least one BCL-2 inhibitor and at least one amount of at least one hypomethylating agent.

The present disclosure provides methods of identifying that a subject having AML will not be responsive to an AML treatment comprising the administration of a combination of at least one BCL-2 inhibitor and at least one hypomethylating agent, the methods comprising: a) determining the mutational status of at least one of KRAS, PTPN11, SMC1A, WT1, SETPB1, JAK2, and p53 in a plurality of cells in a sample from the subject; b) identifying that the subject will not be responsive to the AML treatment when the presence of at least one mutation in at least one of KRAS, PTPN11, SMC1A, WT1. SETPB1, JAK2, and p53 is identified.

The present disclosure provides methods of identifying that a subject having AML will not be responsive to an AML treatment comprising the administration of a combination of at least one BCL-2 inhibitor and at least one hypomethylating agent, the methods comprising: a) measuring the expression of at least one of CD5. CD7, CD16, CD22, CD34, CD36, CD56, and CD71 in a plurality of cells in a sample from the subject; b) identifying the presence of at least one cell that expresses at least one of CD5, CD7, CD16, CD22, CD34, CD36, CD56, and CD71 based on the expression measured in step (a); c) identifying that the subject will not be responsive to the AML treatment when the presence of at cell expressing at least one of CD5, CD7, CD16, CD22, CD34, CD36, CD56, and CD71 is identified.

The present disclosure provides methods of identifying if a subject having AML will not be responsive to an AML treatment comprising the administration of a combination of at least one BCL-2 inhibitor and at least one hypomethylating agent, the methods comprising: a) determining the cytogenetic status of a plurality of cells in a sample from the subject; and b) identifying that the subject will not be responsive to the AML treatment when the presence of at least cell exhibiting at least one of: i) a Del7q phenotype; ii) a Inv3 phenotype; iii) a Complex Cytogenetics phenotype; iv) a translocation t(9; 11) phenotype; v) a 7 centromere loss phenotype; vi) an 8 centromere loss phenotype; vii) a MECOM rearrangement phenotype; and viii) a MLL rearrangement phenotype is identified.

In some aspects, the methods further comprising administering to the subject identified as not being responsive to AML treatment at least one alternative AML treatment, wherein the at least one alternative AML treatment comprises: i) does not comprise a BCL-2 inhibitor in combination with an HMA; or ii) comprises the administration of a combination of at least one BCL-2 inhibitor, at least one HMA, and at least one additional agent.

In some aspects, the at least one additional agent is a CD70-targeting agent.

In some aspects, the at least one CD70-targeting agent comprises: i) an anti-CD70 antibody, preferably wherein the anti-CD70 antibody is cusatuzumab; ii) an anti-CD70 immunotherapy, preferably wherein the immunotherapy comprises CAR-T and/or NK Cells that are directed specifically at CD70; iii) an agent that blocks CD70 signaling, preferably wherein the agent that blocks CD70 signaling prevents binding of CD27 and CD70.

In some aspects, the at least one hypomethylating agent is selected from azacitidine and decitabine.

In some aspects, the at least one BCL-2 inhibitor is selected from venetoclax and navitoclax.

In some aspects, step (a) comprises performing cytogenic analysis, routine karyotyping, fluorescent in situ hybridization (FISH), comparative genomic hybridization (CGH), array comparative genomic hybridization (aCGH). PCR, high-throughput sequencing, next generation sequencing (NGS), Northern Blot, reverse transcription PCR (RT-PCR), real-time PCR (qPCR), quantitative PCR, qRT-PCR, flow cytometry, mass spectrometry, microarray analysis, digital droplet PCR, Western Blot, Cellular Indexing of Transcriptomes and Epitopes by Sequencing (CITE-SEQ), any single-cell omics technology or any combination thereof.

In some aspects, the subject is: i) a subject having AML who has not received any treatment for AML; or ii) a subject having AML who has received previously received at least one AML treatment, preferably wherein the at least one AML treatment comprises a combination of at least one BCL-2 inhibitor and at least one hypomethylating agent.

In some aspects, the subject is: i) a subject that has not received a stem cell transplant; or ii) a subject that has previously received at least one stem cell transplant.

In some aspects, identifying that a subject will be responsive to the AML treatment comprises identifying that the subject will have a durable remission after receiving the AML treatment.

In some aspects, identifying that a subject will not be responsive to the AML treatment comprises identifying that the subject will be refractory to the AML treatment and/or that the subject will suffer a relapse after receiving the AML treatment.

In some aspects, the biological sample comprises blood, a bone marrow biopsy, a bone marrow aspirate, a biopsy of a chloroma, a tissue biopsy, cerebrospinal fluid or any combination thereof.

In some aspects, the plurality of cells comprises acute myeloid leukemia cells.

In some aspects, the acute myeloid leukemia cells comprise acute myeloid leukemia blast cells, leukemia stem cells, or any combination thereof. Any of the above aspects, or any of the aspects described herein, can be combined with any other aspect.

Unless otherwise defined, all technical and scientific terms used herein have the same meaning as commonly understood by one of ordinary skill in the art to which this disclosure belongs. In the Specification, the singular forms also include the plural unless the context clearly dictates otherwise; as examples, the terms “a,” “an,” and “the” are understood to be singular or plural and the term “or” is understood to be inclusive. By way of example, “an element” means one or more element. Throughout the specification the word “comprising,” or variations such as “comprises” or “comprising,” will be understood to imply the inclusion of a stated element, integer or step, or group of elements, integers or steps, but not the exclusion of any other element, integer or step, or group of elements, integers or steps. About can be understood as within 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.” Unless specifically stated or obvious from context, as used herein, the term “or” is understood to be inclusive and covers both “or” and “and”.

Although methods and materials similar or equivalent to those described herein can be used in the practice or testing of the present disclosure, suitable methods and materials are described below. All publications, patent applications, patents, and other references mentioned herein are incorporated by reference in their entirety. The references cited herein are not admitted to be prior art to the claimed invention. In the case of conflict, the present Specification, including definitions, will control. In addition, the materials, methods, and examples are illustrative only and are not intended to be limiting. Other features and advantages of the disclosure will be apparent from the following detailed description and claim.

### BRIEF DESCRIPTION OF THE DRAWINGS

The above and further features will be more clearly appreciated from the following detailed description when taken in conjunction with the accompanying drawings.

FIG. 1 is a graph showing the survival probability of AML subjects exhibiting normal LDH levels or elevated LDH levels after receiving a combination of venetoclax and azacitidine. Elevated LDH levels in FIG. 1 correspond to LDH levels that are greater than the Upper Limit of Normal (ULN) for the laboratory in which the LDH levels were measured.

FIG. 2 is a graph showing the survival probability of AML subjects exhibiting normal LDH levels or elevated LDH levels after receiving a combination of venetoclax, azacitidine and cusatuzumab. Elevated LDH levels in FIG. 2 correspond to LDH levels that are greater than the Upper Limit of Normal (ULN) for the laboratory in which the LDH levels were measured.

FIG. 3 is a graph showing the survival probability of AML subjects exhibiting normal LDH levels or elevated LDH levels after a treatment that includes at least cusatuzumab. Elevated LDH levels in FIG. 3 correspond to LDH levels that are greater than the Upper Limit of Normal (ULN) for the laboratory in which the LDH levels were measured.

FIG. 4 shows the demographics for the subjects used in the study described in Example 2 of the present disclosure.

FIG. 5 shows a flow diagram that summarizes the patient populations in the CU Ven/Aza data set used in Example 2 of the present disclosure and how they were utilized in generating the Ven/aza risk stratification model.

FIG. 6A and FIG. 6B show a graphical representation of statistical methodology discussed in Example 2 of the present disclosure.

FIG. 7A and FIG. 7B show the definitions of risk factor and logic for the Ven/Aza risk model described in Example 2 of the present disclosure, as well as genetic features associated with AML risk groups for Overall Survival (OS) and Event Free Survival (EFS).

FIG. 8A, FIG. 8B, FIG. 8C, FIG. 8D and FIG. 8E show Kaplan Meir analysis of genetic features associated with OS in Ven/Aza treated patients, as described in Example 2 of the present disclosure. FIG. 8A and FIG. 8B show the application of the Ven/Aza specific risk stratification model to the Ven/Aza treated cohort for OS. FIG. 8C shows the application of the ELN 2022 criteria to the Ven/Aza treated cohort. FIG. 8D shows the pairwise comparison between the Ven/Aza specific risk stratification model and the ELN 2022 mode for the adverse, intermediate and adverse risk groups. FIG. 8E shows the comparison of predictive performance stratification of the Ven/Aza and ELN 2022 models for overall survival.

FIG. 9A, FIG. 9B, FIG. 9C, and FIG. 9D show Kaplan Meir analysis of genetic features associated with EFS in Ven/Aza treated patients, as described in Example 2 of the present disclosure. FIG. 9A shows the application of the Ven/Aza specific risk stratification model to the Ven/Aza treated cohort for EFS. FIG. 9B shows the application of the ELN 2022 criteria for EFS. FIG. 9C shows a pairwise comparison between the Ven/Aza specific risk stratification model and the ELN 2022 mode for the adverse, intermediate and adverse risk groups. FIG. 9D shows a comparison of predictive performance stratification of the Ven/Aza and ELN 2022 models for event free survival.

FIG. 10A shows risk stratification of genetic plus flow cytometric features based on ven/aza risk model with respect to OS and EFS under an ensemble approach

FIG. 10B and FIG. 10C show overall survival (FIG. 10B) and event free survival (FIG. 10C) based on favorable, intermediate, and adverse among patients undergoing an SCT.

FIG. 11A and FIG. 11B show overall survival and event free survival based on favorable, intermediate, and adverse genetic features (FIG. 11A) and genetic features plus phenotypic features (FIG. 11B).

FIG. 12 is a series of graphs showing the survival probability of AML subjects exhibiting either a CD2+ AML phenotype or a CD2− AML phenotype after receiving a combination of venetoclax, azacitidine and cusatuzumab.

FIG. 13 is a series of graphs showing the survival probability of AML subjects exhibiting mutated NF1 or wildtype NF1 after receiving a combination of venetoclax, azacitidine and cusatuzumab.

FIG. 14 is a series of graphs showing the survival probability of AML subjects exhibiting mutated TP53 or wildtype TP53 after receiving a combination of venetoclax, azacitidine and cusatuzumab.

FIG. 15 is a series of graphs showing the survival probability of AML subjects exhibiting either a normal chromosome 7 phenotype or a deleted chromosome 7 phenotype after receiving a combination of venetoclax, azacitidine and cusatuzumab.

FIG. 16 is series of graphs showing the survival probability of AML subjects exhibiting a CD7+ AML phenotype or a CD7− AML phenotype after receiving either a combination of venetoclax and azacitidine (top panels) or a combination of venetoclax, azacitidine and cusatuzumab (bottom panels).

FIG. 17 is series of graphs showing the survival probability of AML subjects exhibiting mutated KRAS or wildtype KRAS after receiving either a combination of venetoclax and azacitidine (top panels) or a combination of venetoclax, azacitidine and cusatuzumab (bottom panels).

FIG. 18 is series of graphs showing the survival probability of AML subjects exhibiting mutated PTPN11 or wildtype PTPN11 after receiving either a combination of venctoclax and azacitidine (top panels) or a combination of venctoclax, azacitidine and cusatuzumab (bottom panels).

### DETAILED DESCRIPTION

Acute myeloid leukemia is diagnosed in ~15,000-20,000 persons each year in the US. Treatment for patients who are young and fit typically involves aggressive initial induction with intensive chemotherapy (IC) such as an anthracycline plus cytosine arabinoside followed by additional consolidative chemotherapy or allogeneic stem cell transplant (AlloSCT) for appropriate candidates. For older and less fit patients, the BCL-2 directed agent venetoclax combined with a hypomethylating agent (ven/IMA) such as azacytidine or decitabine has become a new standard of care. For IC type therapies, a variety of prognostic schemes have been developed based on patient and AML features that are associated with longer versus shorter overall survival and other outcomes. The most recent European Leukemia Network 2022 prognostic model divides patients into favorable, intermediate and adverse-risk groups based on AML cytogenetic, fluorescence in situ hybridization (FISH) and next generation sequencing (NGS) features (see Dohner, H., et al. Blood 140, 2022, 1345-1377. Favorable risk factors in AML at diagnosis include t(8; 21)(q22; q22.1)/RUNX1::RUNX1 T1, inv(16)(p13.1q22) or t(16; 16)(p13.1; q22)/CBFB::MYH11, mutated NPM1 without FLT3-ITD and bZIP in-frame mutated CEBPA. Adverse risk factors include t(6; 9)(p23; q34.1)/DEK::NUP214, t(v; 11q23.3)/KMT2A-rearranged, t(9; 22)(q34.1; q11.2)/BCR::ABL1, t(8; 16)(p11; p13)/KAT6A::CREBBP, inv(3)(q21.3q26.2) or t(3; 3)(q21.3; q26.2)/GATA2. MECOM(EVI1), t(3q26.2; v)/MECOM(EVI1)-rearranged, −5 or del(5q); −7; −17/abn(17p), complex karyotype, monosomal karyotype, mutated ASXL1, BCOR, EZH2, RUNX1, SF3B1, SRSF2, STAG2, U2AF1, orZRSR2 and mutated TP53. Approximately 15%, 27%, and 56% of patients were classified as favorable, moderate, or unfavorable risk using these criteria in one study (see da Rosa, S. E. A., et al. Clin Transl Oncol. 2023) and 32%, 27% and 41% respectively in a second study (see Rausch, C., et al. Leukemia, 2023). This second study also analyzed outcomes of 1118 newly diagnosed AML patients receiving cytarabine based induction therapy between 1999-2012 who were classified according to the ELN 2022 prognostic scheme. Associations for the favorable, intermediate and adverse risk groups and 5-year OS of 55%, 34% and 15% were found respectively. A survival predictive model for older patients treated with lower intensity chemotherapy has also been developed using primarily patient features and cytogenetics with 3-year survival rates were 52%, 24%, 10%, and 1% in favorable, intermediate, poor, and very poor risk groups, respectively (see Sasaki, K., et al. Cancer, 129, 2023, 1017-1029). A subset of these patients whose AML underwent mutational profiling identified NPM1 and IDH2 as favorable genetic features and TP53 was found to be unfavorable.

To date, while multiple reports have identified individual patient and AML features associated with outcomes following ven/HMA, no systematic risk stratification model based on AML genetic, phenotypic or other features has been published specifically for elderly/unfit patients treated with ven/HMA as front-line therapy.

Additionally, researchers have also sought to develop additional treatments that can be used in addition to ven/HMA to help patients achieve complete remission. Specifically, antibodies, cellular immunotherapies and other agents directed at CD70 or CD70 biologic processes have recently been developed as an additional therapy to be administered in combination with venetoclax/HMA therapy regimen. These new therapies include cusatuzumab, an anti-CD70 monoclonal antibody that is currently being tested in clinical trials for the treatment of AML in combination with ven/aza.

Thus, there is a need in the art for methods of identifying patients that will not be responsive to treatment with a combination of a BCL-2 inhibitor (e.g. venetoclax) and a HMA (e.g. azacitidine), as well as a need for methods of identifying patients who will benefit from treatment with a combination of a BCL-2 inhibitor (e.g. venetoclax), an HMA (e.g. azacitidine) and a CD70-targeting agent (e.g. cusatuzumab) respond to treatment with Ven/aza. The ability to identify these patients, prior to treatment, would allow clinicians to avoid the toxicity, expense and negative quality of life associated with an ineffective therapy.

The present disclosure is also based on, inter alia, the discovery of several different biomarkers that can be used to predict a subject's response to treatment with a combination of a BCL-2 inhibitor and an HMA.

The present disclosure is also based on, inter alia, the discovery of several different biomarkers that can be used to predict a subject's response to treatment with a CD70-targeting agent, including treatment with a combination of a BCL-2 inhibitor, a hypomethylating agent and a CD70-targeting agent.

Accordingly, the present disclosure provides, inter alia, methods of determining if a subject having AML will be responsive to treatments comprising the administration of a CD-70 targeting agent (including treatments comprising the administration of a combination of at least one BCL-2 inhibitor, at least one hypomethylating agent and at least one CD-70 targeting agent) based on the biomarkers described herein. The present disclosure also provides methods of determining if a subject having AML will be responsive to treatment with a combination of at least one BCL-2 inhibitor and at least one hypomethylating agent based on the biomarkers herein. The subjects identified as not being responsive to the combination of at least one BCL-2 inhibitor and at least one hypomethylating agent can be further identified for future treatment comprising at least one CD-70 targeting agent.

Methods Based on BCORL1, EZH2, IDH1, IDH2, NPM1, RAD21, SF3B1, SH2B3 and/or KIT

The following methods are based, inter alia, on the discovery (presented herein in the experimental examples) that AML subjects having at least one mutation in at least one BCORL1, EZH2, IDH1, IDH2, NPM1, RAD21, SF3B1, SH2B3 and KIT may respond favorably to treatment comprising the administration of a combination of at least one BCL-2 inhibitor and at least one hypomethylating agent. That is, AML subjects having at least one mutation in at least one BCORL1, EZH2, IDH1, IDH2, NPM1, RAD21, SF3B1, SH2B3 and KIT exhibited longer overall survival times after treatment with at least one BCL-2 inhibitor and at least one hypomethylating agent as compared to other AML subjects.

The present disclosure provides methods of identifying that a subject having AML will be responsive to an AML treatment comprising the administration of a combination of at least one BCL-2 inhibitor and at least one hypomethylating agent, the methods comprising: a) determining the mutational status of one or more genes in a plurality of cells in a sample from the subject; b) identifying that the subject will be responsive to the AML treatment when the presence of at least one mutation in at least one of the one or more genes is identified.

The present disclosure provides methods of identifying that a subject having AML will be responsive to an AML treatment comprising the administration of a combination of at least one BCL-2 inhibitor and at least one hypomethylating agent, the methods comprising: a) determining the mutational status of one or more genes in a plurality of cells in a sample from the subject; b) determining the number and/or percentage of cells in the plurality of cells that exhibit at least one mutation in at least one of the one or more genes based on step (a); c) comparing the number and/or percentage identified in step (b) to a predetermined cutoff value; and d) identifying that the subject will be responsive to the AML treatment when the number and/or percentage of cells that exhibit at least one mutation in at least one of the one or more genes is equal to or greater than the predetermined cutoff value.

The present disclosure provides methods of treating AML in a subject, the methods comprising: a) determining the mutational status of one or more genes in a plurality of cells in a sample from the subject; b) administering to the subject a combination comprising at least one BCL-2 inhibitor and at least one hypomethylating agent when the presence of at least one mutation in at least one of the one or more genes is identified.

The present disclosure provides methods of treating AML in a subject, the methods comprising: a) determining the mutational status of one or more genes in a plurality of cells in a sample from the subject; b) determining the number and/or percentage of cells in the plurality of cells that exhibit at least one mutation in at least one of the one or more genes based on step (a); c) comparing the number and/or percentage identified in step (b) to a predetermined cutoff value; and d) administering to the subject a combination comprising at least one BCL-2 inhibitor and at least one hypomethylating agent when the number and/or percentage of cells that exhibit at least one mutation in at least one of the one or more genes is equal to or greater than the predetermined cutoff value.

In some aspects of the preceding methods, the one or more genes can be BCORL1, EZH2, IDH1, IDH2, NPM1, RAD21, SF3B1, SH2B3 and KIT.

In some aspects, the one or more genes can be BCORL1.

In some aspects, the one or more genes can be EZH2.

In some aspects, the one or more genes can be IDH1.

In some aspects, the one or more genes can be IDH2.

In some aspects, the one or more genes can be NPM1.

In some aspects, the one or more genes can be RAD21.

In some aspects, the one or more genes can be SF3B1.

In some aspects, the one or more genes can be SH2B3.

In some aspects, the one or more genes can be KIT.

Methods based on IDH1, IDH2, NPM1, RAD21 and/or KIT

The following methods are based, inter alia, on the discovery (presented herein in the experimental examples) that AML subjects having at least one mutation in at least one IDH1, IDH2, NPM1, RAD21, and KIT may respond favorably to treatment comprising the administration of a combination of at least one BCL-2 inhibitor and at least one hypomethylating agent. That is, AML subjects having at least one mutation in at least one IDH1, IDH2, NPM1, RAD21, and KIT exhibited longer even free survival times after treatment with at least one BCL-2 inhibitor and at least one hypomethylating agent as compared to other AML subjects.

The present disclosure provides methods of identifying that a subject having AML will be responsive to an AML treatment comprising the administration of a combination of at least one BCL-2 inhibitor and at least one hypomethylating agent, the methods comprising: a) determining the mutational status of one or more genes in a plurality of cells in a sample from the subject; b) identifying that the subject will be responsive to the AML treatment when the presence of at least one mutation in at least one of the one or more genes is identified.

The present disclosure provides methods of identifying that a subject having AML will be responsive to an AML treatment comprising the administration of a combination of at least one BCL-2 inhibitor and at least one hypomethylating agent, the methods comprising: a) determining the mutational status of one or more genes in a plurality of cells in a sample from the subject; b) determining the number and/or percentage of cells in the plurality of cells that exhibit at least one mutation in at least one of the one or more genes based on step (a); c) comparing the number and/or percentage identified in step (b) to a predetermined cutoff value; and d) identifying that the subject will be responsive to the AML treatment when the number and/or percentage of cells that exhibit at least one mutation in at least one of the one or more genes is equal to or greater than the predetermined cutoff value.

The present disclosure provides methods of treating AML in a subject, the methods comprising: a) determining the mutational status of one or more genes in a plurality of cells in a sample from the subject; b) administering to the subject a combination comprising at least one BCL-2 inhibitor and at least one hypomethylating agent when the presence of at least one mutation in at least one of the one or more genes is identified.

The present disclosure provides methods of treating AML in a subject, the methods comprising: a) determining the mutational status of one or more genes in a plurality of cells in a sample from the subject; b) determining the number and/or percentage of cells in the plurality of cells that exhibit at least one mutation in at least one of the one or more genes based on step (a); c) comparing the number and/or percentage identified in step (b) to a predetermined cutoff value; and d) administering to the subject a combination comprising at least one BCL-2 inhibitor and at least one hypomethylating agent when the number and/or percentage of cells that exhibit at least one mutation in at least one of the one or more genes is equal to or greater than the predetermined cutoff value.

In some aspects, the one or more genes can be IDH1, IDH2, NPM1, RAD21, and KIT.

In some aspects, the one or more genes can be IDH1.

In some aspects, the one or more genes can be IDH2.

In some aspects, the one or more genes can be NPM1.

In some aspects, the one or more genes can be RAD21.

In some aspects of the preceding methods, the one or more genes can be KIT.

Methods based on KRAS, PTPN11, SMC1A, WT1 and/or p53

The following methods are based, inter alia, on the discovery (presented herein in the experimental examples) that AML subjects having at least one mutation in at least one KRAS, PTPN11, SMC1A, WT1 and p53 may not respond favorably to treatment comprising the administration of a combination of at least one BCL-2 inhibitor and at least one hypomethylating agent. That is, AML subjects having at least one mutation in at least one KRAS, PTPN11, SMC1A, WT1 and p53 exhibited shorter event free survival times after treatment with at least one BCL-2 inhibitor and at least one hypomethylating agent as compared to other AML subjects.

The present disclosure provides methods of identifying that a subject having AML will not be responsive to an AML treatment comprising the administration of a combination of at least one BCL-2 inhibitor and at least one hypomethylating agent, the methods comprising: a) determining the mutational status of one or more genes in a plurality of cells in a sample from the subject; b) identifying that the subject will not be responsive to the AML treatment when the presence of at least one mutation in at least one of the one or more genes is identified.

The present disclosure provides methods of identifying that a subject having AML will not be responsive to an AML treatment comprising the administration of a combination of at least one BCL-2 inhibitor and at least one hypomethylating agent, the methods comprising: a) determining the mutational status of one or more genes in a plurality of cells in a sample from the subject; b) determining the number and/or percentage of cells in the plurality of cells that exhibit at least one mutation in at least one of the one or more genes based on step (a); c) comparing the number and/or percentage identified in step (b) to a predetermined cutoff value; and d) identifying that the subject will not be responsive to the AML treatment when the number and/or percentage of cells that exhibit at least one mutation in at least one of the one or more genes is equal to or greater than the predetermined cutoff value.

The present disclosure provides methods of treating AML in a subject, the methods comprising: a) determining the mutational status of one or more genes in a plurality of cells in a sample from the subject; b) administering to the subject an alternative AML treatment when the presence of at least one mutation in at least one of the one or more genes is identified.

The present disclosure provides methods of treating AML in a subject, the methods comprising: a) determining the mutational status of one or more genes in a plurality of cells in a sample from the subject; b) determining the number and/or percentage of cells in the plurality of cells that exhibit at least one mutation in at least one of the one or more genes based on step (a); c) comparing the number and/or percentage identified in step (b) to a predetermined cutoff value; and d) administering to the subject an alternative AML treatment when the number and/or percentage of cells that exhibit at least one mutation in at least one of the one or more genes is equal to or greater than the predetermined cutoff value.

In some aspects of the preceding methods, an alternative AML treatment is a treatment that does not comprise a BCL-2 inhibitor in combination with an HMA.

In some aspects of the preceding methods, an alternative AML treatment is a treatment that comprises the administration of a combination of at least one BCL-2 inhibitor, at least one HMA, and at least one additional agent. In some aspects, the at least one additional agent is a CD70-targeting agent.

In some aspects, the one or more genes can be KRAS, PTPN11, SMC1A, WT1 and p53.

In some aspects, the one or more genes can be KRAS.

In some aspects, the one or more genes can be PTPN11.

In some aspects, the one or more genes can be SMC1A.

In some aspects, the one or more genes can be WT1.

In some aspects, the one or more genes can be p53.

Methods based on KRAS, PTPN11, SMC1A, SETPB1, JAK2 and/or p53m

The following methods are based, inter alia, on the discovery (presented herein in the experimental examples) that AML subjects having at least one mutation in at least one KRAS, PTPN11, SMC1A, SETPB1, JAK2 and p53 may not respond favorably to treatment comprising the administration of a combination of at least one BCL-2 inhibitor and at least one hypomethylating agent. That is, AML subjects having at least one mutation in at least one KRAS, PTPN11, SMC1A, WT1 and p53 exhibited shorter overall survival times after treatment with at least one BCL-2 inhibitor and at least one hypomethylating agent as compared to other AML subjects.

The present disclosure provides methods of identifying that a subject having AML will not be responsive to an AML treatment comprising the administration of a combination of at least one BCL-2 inhibitor and at least one hypomethylating agent, the methods comprising: a) determining the mutational status of one or more genes in a plurality of cells in a sample from the subject; b) identifying that the subject will not be responsive to the AML treatment when the presence of at least one mutation in at least one of the one or more genes is identified.

The present disclosure provides methods of identifying that a subject having AML will not be responsive to an AML treatment comprising the administration of a combination of at least one BCL-2 inhibitor and at least one hypomethylating agent, the methods comprising: a) determining the mutational status of one or more genes in a plurality of cells in a sample from the subject; b) determining the number and/or percentage of cells in the plurality of cells that exhibit at least one mutation in at least one of the one or more genes based on step (a); c) comparing the number and/or percentage identified in step (b) to a predetermined cutoff value; and d) identifying that the subject will not be responsive to the AML treatment when the number and/or percentage of cells that exhibit at least one mutation in at least one of the one or more genes is equal to or greater than the predetermined cutoff value.

The present disclosure provides methods of treating AML in a subject, the methods comprising: a) determining the mutational status of one or more genes in a plurality of cells in a sample from the subject; b) administering to the subject an alternative AML treatment when the presence of at least one mutation in at least one of the one or more genes is identified.

The present disclosure provides methods of treating AML in a subject, the methods comprising: a) determining the mutational status of one or more genes in a plurality of cells in a sample from the subject; b) determining the number and/or percentage of cells in the plurality of cells that exhibit at least one mutation in at least one of the one or more genes based on step (a); c) comparing the number and/or percentage identified in step (b) to a predetermined cutoff value; and d) administering to the subject an alternative AML treatment when the number and/or percentage of cells that exhibit at least one mutation in at least one of the one or more genes is equal to or greater than the predetermined cutoff value.

In some aspects of the preceding methods, an alternative AML treatment is a treatment that does not comprise a BCL-2 inhibitor in combination with an HMA.

In some aspects of the preceding methods, an alternative AML treatment is a treatment that comprises the administration of a combination of at least one BCL-2 inhibitor, at least one HMA, and at least one additional agent. In some aspects, the at least one additional agent is a CD70-targeting agent.

In some aspects of the preceding methods, the one or more genes can be KRAS, PTPN11, SMC1A, SETPB1, JAK2 and p53.

In some aspects, the one or more genes can be KRAS.

In some aspects, the one or more genes can be PTPN11.

In some aspects, the one or more genes can be SMC1A.

In some aspects, the one or more genes can be SETPB1.

In some aspects, the one or more genes can be JAK2.

In some aspects, the one or more genes can p53.

### Methods Based on CD10

The following methods are based, inter alia, on the discovery (presented herein in the experimental examples) that AML subjects having CD10+ phenotypes may respond to treatment comprising the administration of a combination of at least one BCL-2 inhibitor and at least one hypomethylating agent. That is, AML subjects having a CD10+ phenotype exhibited longer overall survival times and longer event free survival times after treatment with at least one BCL-2 inhibitor and at least one hypomethylating agent as compared to other AML subjects.

The present disclosure provides methods of identifying that a subject having AML will be responsive to an AML treatment comprising the administration of a combination of at least one BCL-2 inhibitor and at least one hypomethylating agent, the methods comprising: a) measuring the expression of at least CD10 in a plurality of cells in a sample from the subject; b) identifying the presence of at least one CD10+ cell based on the expression measured in step (a); c) identifying that the subject will be responsive to the AML treatment when the presence of at CD10+ cell is identified.

The present disclosure provides methods of identifying that a subject having AML will be responsive to an AML treatment comprising the administration of a combination of at least one BCL-2 inhibitor and at least one hypomethylating agent, the methods comprising: a) measuring the expression of at least CD10 in a plurality of cells in a sample from the subject; b) identifying the number and/or percentage of CD10+ cells in the plurality of cells based on the expression measured in step (a); c) comparing the number and/or percentage of CD10+ cells identified in step (b) to a predetermined cutoff value; and d) identifying that the subject will be responsive to the AML treatment when the number and/or percentage CD10+ cells is equal to or greater than the predetermined cutoff value.

The present disclosure provides methods of treating AML in a subject, the methods comprising: a) measuring the expression of at least CD10 in a plurality of cells in a sample from the subject; b) identifying the presence of at least one CD10+ cell based on the expression measured in step (a); c) administering to the subject a combination comprising at least one BCL-2 inhibitor and at least one hypomethylating agent when at least one CD10+ cell is identified.

The present disclosure provides methods of identifying that a subject having AML will be responsive to an AML treatment comprising the administration of a combination of at least one BCL-2 inhibitor and at least one hypomethylating agent, the methods comprising: a) measuring the expression of at least CD10 in a plurality of cells in a sample from the subject; b) identifying the number and/or percentage of CD10+ cells in the plurality of cells based on the expression measured in step (a); c) comparing the number and/or percentage of CD10+ cells identified in step (b) to a predetermined cutoff value; and d) administering to the subject a combination comprising at least one BCL-2 inhibitor and at least one hypomethylating agent when the number and/or percentage CD10+ cells is equal to or greater than the predetermined cutoff value.

Methods Based on CD5, CD7, CD16, CD36, CD56 and/or CD71

The following methods are based, inter alia, on the discovery (presented herein in the experimental examples) that AML subjects having CD5+, CD7+, CD16+, CD36+, CD56+ and/or CD71+ phenotypes may not respond favorably to treatment comprising the administration of a combination of at least one BCL-2 inhibitor and at least one hypomethylating agent. That is, AML subjects having a CD5+, CD7+, CD16+, CD36+, CD56+ and/or CD71+ phenotypes exhibited shorter overall survival times after treatment with at least one BCL-2 inhibitor and at least one hypomethylating agent as compared to other AML subjects.

The present disclosure provides methods of identifying that a subject having AML will not be responsive to an AML treatment comprising the administration of a combination of at least one BCL-2 inhibitor and at least one hypomethylating agent, the methods comprising: a) measuring the expression of at least one cell surface biomarker in a plurality of cells in a sample from the subject; b) identifying the presence of at least one cell that expresses at least one of the cell surface biomarkers based on the expression measured in step (a); c) identifying that the subject will not be responsive to the AML treatment when the presence of at cell expressing at least one of the cell surface biomarkers is identified.

The present disclosure provides methods of identifying that a subject having AML will not be responsive to an AML treatment comprising the administration of a combination of at least one BCL-2 inhibitor and at least one hypomethylating agent, the methods comprising: a) measuring the expression of at least one cell surface biomarker in a plurality of cells in a sample from the subject; b) identifying the number and/or percentage of cells in the plurality of cells that express at least one of the cell surface biomarkers based on the expression measured in step (a); c) comparing the number and/or percentage of cells identified in step (b) to a predetermined cutoff value; and d) identifying that the subject will not be responsive to the AML treatment when the number and/or percentage cells identified in step (b) is equal to or greater than the predetermined cutoff value.

The present disclosure provides methods of treating AML in a subject, the methods comprising: a) measuring the expression of at least one cell surface biomarker in a plurality of cells in a sample from the subject; b) identifying the presence of at least one cell that expresses at least one of the cell surface biomarkers based on the expression measured in step (a); c) administering to the subject an alternative AML treatment when the presence of at cell expressing at least one of the cell surface biomarkers is identified.

The present disclosure provides methods treating AML in a subject, the methods comprising: a) measuring the expression of at least one cell surface biomarker in a plurality of cells in a sample from the subject; b) identifying the number and/or percentage of cells in the plurality of cells that express at least one of the cell surface biomarkers based on the expression measured in step (a); c) comparing the number and/or percentage of cells identified in step (b) to a predetermined cutoff value; and d) administering to the subject an alternative AML treatment when the number and/or percentage cells identified in step (b) is equal to or greater than the predetermined cutoff value.

In some aspects of the preceding methods, the at least one cell surface biomarker comprises CD5, CD7, CD16, CD36, CD56, and CD71.

In some aspects of the preceding methods, the at least one cell surface biomarker is CD5.

In some aspects of the preceding methods, the at least one cell surface biomarker is CD7.

In some aspects of the preceding methods, the at least one cell surface biomarker is CD16.

In some aspects of the preceding methods, the at least one cell surface biomarker is CD36.

In some aspects of the preceding methods, the at least one cell surface biomarker is CD56.

In some aspects of the preceding methods, the at least one cell surface biomarker is CD71.

In some aspects of the preceding methods, an alternative AML treatment is a treatment that does not comprise a BCL-2 inhibitor in combination with an HMA.

In some aspects of the preceding methods, an alternative AML treatment is a treatment that comprises the administration of a combination of at least one BCL-2 inhibitor, at least one HMA, and at least one additional agent. In some aspects, the at least one additional agent is a CD70-targeting agent.

Methods Based on CD16, CD22, CD34, CD36, CD56 and/or CD71

The following methods are based, inter alia, on the discovery (presented herein in the experimental examples) that AML subjects having CD16+, CD22+, CD34+, CD36+, CD56+ and/or CD71+ phenotypes may not respond favorably to treatment comprising the administration of a combination of at least one BCL-2 inhibitor and at least one hypomethylating agent. That is, AML subjects having a CD16+, CD22+, CD34+, CD36+, CD56+ and/or CD71+ phenotypes exhibited shorter event free survival times after treatment with at least one BCL-2 inhibitor and at least one hypomethylating agent as compared to other AML subjects.

The present disclosure provides methods of identifying that a subject having AML will not be responsive to an AML treatment comprising the administration of a combination of at least one BCL-2 inhibitor and at least one hypomethylating agent, the methods comprising: a) measuring the expression of at least one cell surface biomarker in a plurality of cells in a sample from the subject; b) identifying the presence of at least one cell that expresses at least one of the cell surface biomarkers based on the expression measured in step (a); c) identifying that the subject will not be responsive to the AML treatment when the presence of at cell expressing at least one of the cell surface biomarkers is identified.

The present disclosure provides methods of identifying that a subject having AML will not be responsive to an AML treatment comprising the administration of a combination of at least one BCL-2 inhibitor and at least one hypomethylating agent, the methods comprising: a) measuring the expression of at least one cell surface biomarker in a plurality of cells in a sample from the subject; b) identifying the number and/or percentage of cells in the plurality of cells that express at least one of the cell surface biomarkers based on the expression measured in step (a); c) comparing the number and/or percentage of cells identified in step (b) to a predetermined cutoff value; and d) identifying that the subject will not be responsive to the AML treatment when the number and/or percentage cells identified in step (b) is equal to or greater than the predetermined cutoff value.

The present disclosure provides methods of treating AML in a subject, the methods comprising: a) measuring the expression of at least one cell surface biomarker in a plurality of cells in a sample from the subject; b) identifying the presence of at least one cell that expresses at least one of the cell surface biomarkers based on the expression measured in step (a); c) administering to the subject an alternative AML treatment when the presence of at cell expressing at least one of the cell surface biomarkers is identified.

The present disclosure provides methods of treating AML in a subject, the methods comprising: a) measuring the expression of at least one cell surface biomarker in a plurality of cells in a sample from the subject; b) identifying the number and/or percentage of cells in the plurality of cells that express at least one of the cell surface biomarkers based on the expression measured in step (a); c) comparing the number and/or percentage of cells identified in step (b) to a predetermined cutoff value; and d) administering to the subject an alternative AML treatment when the number and/or percentage cells identified in step (b) is equal to or greater than the predetermined cutoff value.

In some aspects of the preceding methods, the at least one cell surface biomarker comprises CD16, CD22, CD34, CD36, CD56 and CD71.

In some aspects, the at least one cell surface biomarker is CD16.

In some aspects, the at least one cell surface biomarker is CD22.

In some aspects, the at least one cell surface biomarker is CD34.

In some aspects, the at least one cell surface biomarker is CD36.

In some aspects, the at least one cell surface biomarker is CD56.

In some aspects, the at least one cell surface biomarker is CD71.

In some aspects of the preceding methods, an alternative AML treatment is a treatment that does not comprise a BCL-2 inhibitor in combination with an HMA.

In some aspects of the preceding methods, an alternative AML treatment is a treatment that comprises the administration of a combination of at least one BCL-2 inhibitor, at least one HMA, and at least one additional agent. In some aspects, the at least one additional agent is a CD70-targeting agent.

Methods based on Del5q, CBFB Loss, and/or Normal Cytogenetic Phenotypes

The following methods are based, inter alia, on the discovery (presented herein in the experimental examples) that AML subjects having Del5q, CBFB Loss, and/or Normal Cytogenetic phenotypes may respond favorably to treatment comprising the administration of a combination of at least one BCL-2 inhibitor and at least one hypomethylating agent. That is, AML subjects having a Del5q, CBFB Loss, and/or Normal Cytogenetic phenotype exhibited longer event free survival times after treatment with at least one BCL-2 inhibitor and at least one hypomethylating agent as compared to other AML subjects.

The present disclosure also provides methods of identifying if a subject having AML will be responsive to an AML treatment comprising the administration of a combination of at least one BCL-2 inhibitor and at least one hypomethylating agent, the methods comprising: a) determining the cytogenetic status of a plurality of cells in a sample from the subject; and b) identifying that the subject will be responsive to the AML treatment when the presence of at least cell exhibiting at least one of: i) a Del5q phenotype; ii) a CBFB Loss phenotype; and iii) a Normal Cytogenetics phenotype is identified.

The present disclosure also provides methods of identifying if a subject having AML will be responsive to an AML treatment comprising the administration of a combination of at least one BCL-2 inhibitor and at least one hypomethylating agent, the methods comprising: a) determining the cytogenetic status of a plurality of cells in a sample from the subject; b) determining the number and/or percentage of cells in the plurality of cells that exhibit at least one of: i) a Del5q phenotype; ii) a CBFB Loss phenotype; and iii) a Normal Cytogenetics phenotype, based on step (a); c) comparing the number and/or percentage identified in step (b) to a predetermined cutoff value; and d) identifying that the subject will be responsive to the AML treatment when the number and/or percentage of cells identified in step (b) is equal to or greater than the predetermined cutoff value.

The present disclosure also provides methods of treating AML in a subject, the methods comprising: a) determining the cytogenetic status of a plurality of cells in a sample from the subject; and b) administering to the subject a combination of at least one BCL-2 inhibitor and at least one hypomethylating agent when the presence of at least cell exhibiting at least one of: i) a Del5q phenotype; ii) a CBFB Loss phenotype; and iii) a Normal Cytogenetics phenotype is identified.

The present disclosure also provides methods of treating AML in a subject, the methods comprising: a) determining the cytogenetic status of a plurality of cells in a sample from the subject; b) determining the number and/or percentage of cells in the plurality of cells that exhibit at least one of: i) a Del5q phenotype; ii) a CBFB Loss phenotype; and iii) a Normal Cytogenetics phenotype, based on step (a); c) comparing the number and/or percentage identified in step (b) to a predetermined cutoff value; and d) administering to the subject a combination of at least one BCL-2 inhibitor and at least one hypomethylating agent when the number and/or percentage of cells identified in step (b) is equal to or greater than the predetermined cutoff value.

Methods based on Del7q, Inv3, and/or Complex Cytogenetic Phenotypes

The following methods are based, inter alia, on the discovery (presented herein in the experimental examples) that AML subjects having Del7q, Inv3, and/or Complex Cytogenetic phenotypes may not respond favorably to treatment comprising the administration of a combination of at least one BCL-2 inhibitor and at least one hypomethylating agent. That is, AML subjects having a Del7q, Inv3, and/or Complex Cytogenetic phenotype exhibited shorter overall survival times after treatment with at least one BCL-2 inhibitor and at least one hypomethylating agent as compared to other AML subjects.

The present disclosure also provides methods of identifying if a subject having AML will not be responsive to an AML treatment comprising the administration of a combination of at least one BCL-2 inhibitor and at least one hypomethylating agent, the methods comprising: a) determining the cytogenetic status of a plurality of cells in a sample from the subject; and b) identifying that the subject will not be responsive to the AML treatment when the presence of at least cell exhibiting at least one of: i) a Del7q phenotype; ii) a Inv3 phenotype; and iii) a Complex Cytogenetics phenotype is identified.

The present disclosure also provides methods of identifying if a subject having AML will not be responsive to an AML treatment comprising the administration of a combination of at least one BCL-2 inhibitor and at least one hypomethylating agent, the methods comprising: a) determining the cytogenetic status of a plurality of cells in a sample from the subject; b) determining the number and/or percentage of cells in the plurality of cells that exhibit at least one of: i) a Del7q phenotype; ii) a Inv3 phenotype; and iii) a Complex Cytogenetics phenotype, based on step (a); c) comparing the number and/or percentage identified in step (b) to a predetermined cutoff value; and d) identifying that the subject will not be responsive to the AML treatment when the number and/or percentage of cells identified in step (b) is equal to or greater than the predetermined cutoff value.

The present disclosure also provides methods of treating AML in a subject, the methods comprising: a) determining the cytogenetic status of a plurality of cells in a sample from the subject; and b) administering to the subject an alternative AML treatment when the presence of at least cell exhibiting at least one of: i) a Del7q phenotype; ii) a Inv3 phenotype; and iii) a Complex Cytogenetics phenotype is identified.

The present disclosure also provides methods of treating AML in a subject, the methods comprising: a) determining the cytogenetic status of a plurality of cells in a sample from the subject; b) determining the number and/or percentage of cells in the plurality of cells that exhibit at least one of: i) a Del7q phenotype; ii) a Inv3 phenotype; and iii) a Complex Cytogenetics phenotype, based on step (a); c) comparing the number and/or percentage identified in step (b) to a predetermined cutoff value; and d) administering to the subject an alternative AML treatment when the number and/or percentage of cells identified in step (b) is equal to or greater than the predetermined cutoff value.

In some aspects of the preceding methods, an alternative AML treatment is a treatment that does not comprise a BCL-2 inhibitor in combination with an HMA.

In some aspects of the preceding methods, an alternative AML treatment is a treatment that comprises the administration of a combination of at least one BCL-2 inhibitor, at least one HMA, and at least one additional agent. In some aspects, the at least one additional agent is a CD70-targeting agent.

Methods Based on Del7q, Inv3, Translocation t(9; 11), Complex Cytogenetic, 7 Centromere Loss, 8 Centromere Loss, MECOM Rearrangement and/or MLL Rearrangement Phenotypes

The following methods are based, inter alia, on the discovery (presented herein in the experimental examples) that AML subjects having Del7q, Inv3, translocation t(9; 11), Complex Cytogenetic, 7 centromere loss, 8 centromere loss, MECOM rearrangement and/or MLL rearrangement phenotypes may not respond favorably to treatment comprising the administration of a combination of at least one BCL-2 inhibitor and at least one hypomethylating agent. That is, AML subjects having a Del7q, Inv3, translocation t(9; 11), Complex Cytogenetic, 7 centromere loss, 8 centromere loss, MECOM rearrangement and/or MLL rearrangement phenotypes exhibited shorter event free survival times after treatment with at least one BCL-2 inhibitor and at least one hypomethylating agent as compared to other AML subjects.

The present disclosure also provides methods of identifying if a subject having AML will not be responsive to an AML treatment comprising the administration of a combination of at least one BCL-2 inhibitor and at least one hypomethylating agent, the methods comprising: a) determining the cytogenetic status of a plurality of cells in a sample from the subject; and b) identifying that the subject will not be responsive to the AML treatment when the presence of at least cell exhibiting at least one of: i) a Del7q phenotype; ii) a Inv3 phenotype; iii) a Complex Cytogenetics phenotype; iv) a translocation t(9; 11) phenotype; v) a 7 centromere loss phenotype; vi) an 8 centromere loss phenotype; vii) a MECOM rearrangement phenotype; and viii) a MLL rearrangement phenotype is identified.

The present disclosure also provides methods of identifying if a subject having AML will not be responsive to an AML treatment comprising the administration of a combination of at least one BCL-2 inhibitor and at least one hypomethylating agent, the methods comprising: a) determining the cytogenetic status of a plurality of cells in a sample from the subject; b) determining the number and/or percentage of cells in the plurality of cells that exhibit at least one of: i) a Del7q phenotype; ii) a Inv3 phenotype; iii) a Complex Cytogenetics phenotype; iv) a translocation t(9:11) phenotype; v) a 7 centromere loss phenotype; vi) an 8 centromere loss phenotype; vii) a MECOM rearrangement phenotype; and viii) a MLL rearrangement phenotype, based on step (a); c) comparing the number and/or percentage identified in step (b) to a predetermined cutoff value; and d) identifying that the subject will not be responsive to the AML treatment when the number and/or percentage of cells identified in step (b) is equal to or greater than the predetermined cutoff value.

The present disclosure also provides methods of treating AML in a subject, the methods comprising: a) determining the cytogenetic status of a plurality of cells in a sample from the subject; and b) administering to the subject an alternative AML treatment when the presence of at least cell exhibiting at least one of: i) a Del7q phenotype; ii) a Inv3 phenotype; iii) a Complex Cytogenetics phenotype; iv) a translocation t(9; 11) phenotype; v) a 7 centromere loss phenotype; vi) an 8 centromere loss phenotype; vii) a MECOM rearrangement phenotype; and viii) a MLL rearrangement phenotype is identified.

The present disclosure also provides methods of treating AML in a subject, the methods comprising: a) determining the cytogenetic status of a plurality of cells in a sample from the subject; b) determining the number and/or percentage of cells in the plurality of cells that exhibit at least one of: i) a Del7q phenotype; ii) a Inv3 phenotype; iii) a Complex Cytogenetics phenotype; iv) a translocation t(9; 11) phenotype; v) a 7 centromere loss phenotype; vi) an 8 centromere loss phenotype; vii) a MECOM rearrangement phenotype; and viii) a MLL rearrangement phenotype, based on step (a); c) comparing the number and/or percentage identified in step (b) to a predetermined cutoff value; and d) administering to the subject an alternative AML treatment when the number and/or percentage of cells identified in step (b) is equal to or greater than the predetermined cutoff value.

In some aspects of the preceding methods, an alternative AML treatment is a treatment that does not comprise a BCL-2 inhibitor in combination with an HMA.

In some aspects of the preceding methods, an alternative AML treatment is a treatment that comprises the administration of a combination of at least one BCL-2 inhibitor, at least one HMA, and at least one additional agent. In some aspects, the at least one additional agent is a CD70-targeting agent.

In some aspects of the methods of the present disclosure, a subject that is predicted to be responsive to a particular treatment is a subject that is predicted to have: i) a complete remission (CR) following treatment; ii) a complete remission with partial hematological recovery (CRh) following treatment; or iii) either a CR or a CRh following treatment.

In some aspects of the methods of the present disclosure, a subject that is predicted to not be responsive to a particular treatment is a subject that is predicted to have: i) morphologic leukemia free state (MLSF); ii) partial remission (PR); iii) no response (NR); iv) relapse; or v) any one of MLSF, PR, NR, or relapse.

In methods of the present disclosure wherein a subject is identified as being responsive to a particular treatment (e.g. a combination of a BCL-2 inhibitor and an HMA), the methods can further comprising administering said treatment to the subject.

In methods of the present disclosure wherein a subject is identified as being responsive to a particular treatment (e.g. a combination of a BCL-2 inhibitor and an HMA), the methods can further comprise providing a treatment recommendation to the subject that comprises the administration of said treatment.

In methods of the present disclosure wherein a subject is identified as not responding favorably to a treatment with a combination of a BCL-2 inhibitor and an HMA, the methods can further comprise treating the subject with at least one alternative AML therapy. In some aspects, an alternative AML treatment is a treatment that does not comprise a BCL-2 inhibitor in combination with an HMA. In some aspects, an alternative AML treatment is a treatment that comprises the administration of a combination of at least one BCL-2 inhibitor, at least one HMA, and at least one additional agent. In some aspects, the at least one additional agent is a CD70-targeting agent.

### LDH-Based Methods

The following methods are based, inter alia, on the discovery that AML subjects having elevated LDH levels may respond more favorably to treatment that comprises the administration of at least one CD70-targeting agent as opposed to a treatment that does not comprise the administration of at least one CD70-targeting agent.

Accordingly, the present disclosure provides methods of treating acute myeloid leukemia (AML) in a subject, the methods comprising: a) measuring the LDH level of the subject; b) comparing the subject's LDH level to a predetermined cutoff value; and c) administering to the subject a first treatment when the subject's LDH level is equal to or greater than a predetermined cutoff value, wherein the first treatment comprises at least one CD70-targeting agent. In some aspects, the method further comprises administering to the subject a second treatment when the subject's LDH level is less than the predetermined cutoff value, wherein the second treatment does not comprise a CD70-targeting agent.

The present disclosure also provides methods of identifying if a subject having AML will be responsive to an AML treatment comprising the administration of at least one CD70-targeting agent, the methods comprising: a) measuring the LDH level of the subject; b) comparing the subject's LDH level to a predetermined cutoff value; and c) identifying that the subject will be responsive to the AML treatment when the subject's LDH level is equal to or greater than the predetermined cutoff value.

The present disclosure provides methods of treating AML in a subject in need thereof, the methods comprising administering at least one CD70-targeting agent to the subject, wherein the subject has an LDH level that is equal to or greater than a predetermine cutoff value. In some aspects, the preceding methods can further comprise administering to the subject a combination of at least one BCL-2 inhibitor and at least one hypomethylating agent.

In some aspects, the predetermined cutoff value can be the upper limit of normal for the laboratory which is performing the LDH measurements.

### CD56-Based Methods

The following methods are based, inter alia, on the discovery that AML subjects having CD56+ phenotypes may respond more favorably to treatment that comprises the administration of at least one CD70-targeting agent as opposed to a treatment that does not comprise the administration of at least one CD70-targeting agent.

Accordingly, the present disclosure provides methods of treating acute myeloid leukemia (AML) in a subject, the methods comprising: a) measuring the expression of at least CD56 in a plurality of cells in a sample from the subject; b) identifying the presence of at least one CD56+ cell based on the expression measured in step (a); and c) administering to the subject a first treatment when CD56+ cells are identified, wherein the first treatment comprises at least one CD70-targeting agent. In some aspects, the method further comprises administering to the subject a second treatment when no CD56+ cells are identified, wherein the second treatment does not comprise a CD70-targeting agent.

The present disclosure also provides methods of treating acute myeloid leukemia (AML) in a subject, the methods comprising: a) measuring the expression of at least CD56 in a plurality of cells in a sample from the subject; b) identifying the number and/or percentage of CD56+ cells in the plurality of cells based on the expression measured in step (a); c) comparing the number and/or percentage of CD56+ cells identified in step (b) to a predetermined cutoff value; and d) administering to the subject a first treatment when the number and/or percentage of cells is equal to or greater than the predetermined cutoff value, wherein the first treatment comprises at least one CD70-targeting agent. In some aspects, the method further comprises administering to the subject a second treatment when number and/or percentage of the CD56+ cells is less than the predetermined cutoff value, wherein the second treatment does not comprise a CD70-targeting agent.

The present disclosure also provides methods of identifying if a subject having AML will be responsive to an AML treatment comprising the administration of at least one CD70-targeting agent, the methods comprising: a) measuring the expression of at least CD56 in a plurality of cells in a sample from the subject; b) identifying the presence of at least one CD56+ cell based on the expression measured in step (a); and c) identifying that the subject will be responsive to the AML treatment when CD56+ cells are identified.

The present disclosure also provides methods of identifying if a subject having AML will be responsive to an AML treatment comprising the administration of at least one CD70-targeting agent, the methods comprising: a) measuring the expression of at least CD56 in a plurality of cells in a sample from the subject; b) identifying the number and/or percentage of CD56+ cells in the plurality of cells based on the expression measured in step (a); c) comparing the number and/or percentage of CD56+ cells identified in step (b) to a predetermined cutoff value; and d) identifying that the subject will be responsive to the AML treatment when the number and/or percentage of CD56+ cells is equal to or greater than the predetermined cutoff value.

The present disclosure provides methods of treating AML in a subject in need thereof, the methods comprising administering at least one CD70-targeting agent to the subject, wherein the subject has at least one AML cell that is CD56+. The present disclosure provides methods of treating AML in a subject in need thereof, the methods comprising administering at least one CD70-targeting agent to the subject, wherein the subject has a number and/or percentage of CD56+ AML cells that is equal to or greater than a predetermined cutoff value.

In some aspects, the preceding methods can further comprise administering to the subject a combination of at least one BCL-2 inhibitor and at least one hypomethylating agent.

### NPM1-Based Methods

The following methods are based, inter alia, on the discovery (presented herein in the experimental examples) that AML subjects having mutated NPM1 may respond favorably to treatments that do not comprise the administration of at least one CD70-targeting agent, and therefore said subjects can be excluded from treatment with a CD70-targeting agent.

Accordingly, the present disclosure provides methods of treating acute myeloid leukemia (AML) in a subject, the methods comprising: a) determining the mutational status of NPM1 in a plurality of cells in a sample from the subject; and b) administering to the subject a first treatment when at least one mutation in NPM1 are identified, wherein the first treatment does not comprise at least one CD70-targeting agent. In some aspects, the method can further comprise administering to the subject a second treatment when no mutations in NPM1 are identified, wherein the second treatment comprises at least one CD70-targeting agent.

The present disclosure also provides methods of treating acute myeloid leukemia (AML) in a subject, the methods comprising: a) determining the mutational status of NPM1 in a plurality of cells in a sample from the subject; b) determining the number and/or percentage of cells in the plurality of cells that exhibit at least one mutation in NPM1 based on step (a); c) comparing the number and/or percentage identified in step (b) to a predetermined cutoff value; and d) administering to the subject a first treatment when the number and/or percentage of cells that exhibit at least one mutation in NPM1 is equal to or greater than the predetermined cutoff value, wherein the first treatment does not comprise at least one CD70-targeting agent. In some aspects, the method further comprises administering to the subject a second treatment when the number and/or percentage of cells that exhibit at least one mutation in NPM1 is less than the predetermined cutoff value, wherein the second treatment comprises at least one CD70-targeting agent.

The present disclosure also provides methods of identifying if a subject having AML will be responsive to an AML treatment comprising the administration of at least one CD70-targeting agent, the methods comprising: a) determining the mutational status of NPM1 in a plurality of cells in a sample from the subject; and b) identifying that the subject will be responsive to the AML treatment when no mutations in NPM1 are identified.

The present disclosure also provides methods of identifying if a subject having AML will be responsive to an AML treatment comprising the administration of at least one CD70-targeting agent, the methods comprising: a) determining the mutational status of NPM1 in a plurality of cells in a sample from the subject; b) determining the number and/or percentage of cells in the plurality of cells that exhibit at least one mutation in NPM1 based on step (a); c) comparing the number and/or percentage identified in step (b) to a predetermined cutoff value; and d) identifying that the subject will be responsive to the AML treatment when the number and/or percentage of cells that exhibit at least one mutation in NPM1 is less than the predetermined cutoff value.

The present disclosure provides methods of treating AML in a subject in need thereof, the methods comprising administering at least one CD70-targeting agent to the subject, wherein the subject has no AML cells that exhibit a mutation in NPM1. The present disclosure provides methods of treating AML in a subject in need thereof, the methods comprising administering at least one CD70-targeting agent to the subject, wherein the subject has a number and/or percentage of AML cells that exhibit a mutation in NPM1 that is less than a predetermined cutoff value.

In some aspects, the preceding methods can further comprise administering to the subject a combination of at least one BCL-2 inhibitor and at least one hypomethylating agent. For example, treatments that do not comprise CD-70 targeting agents can comprise at least one BCL-2 inhibitor and at least one hypomethylating agent.

### IDH1-Based Methods

The following methods are based, inter alia, on the discovery (presented herein in the experimental examples) that AML subjects having mutated IDH1 may respond favorably to treatments that do not comprise the administration of at least one CD70-targeting agent, and therefore said subjects can be excluded from treatment with a CD70-targeting agent.

Accordingly, the present disclosure provides methods of treating acute myeloid leukemia (AML) in a subject, the methods comprising: a) determining the mutational status of IDH1 in a plurality of cells in a sample from the subject; and b) administering to the subject a first treatment when at least one mutation in IDH1 are identified, wherein the first treatment does not at least one CD70-targeting agent. In some aspects, the method can further comprise administering to the subject a second treatment when no mutations in IDH1 are identified, wherein the second treatment comprises at least one CD70-targeting agent.

The present disclosure also provides methods of treating acute myeloid leukemia (AML) in a subject, the methods comprising: a) determining the mutational status of IDH1 in a plurality of cells in a sample from the subject; b) determining the number and/or percentage of cells in the plurality of cells that exhibit at least one mutation in IDH1 based on step (a); c) comparing the number and/or percentage identified in step (b) to a predetermined cutoff value; and d) administering to the subject a first treatment when the number and/or percentage of cells that exhibit at least one mutation in IDH1 is equal to or greater than the predetermined cutoff value, wherein the first treatment does not comprise at least one CD70-targeting agent. In some aspects, the method can further comprise administering to the subject a second treatment when the number and/or percentage of cells that exhibit at least one mutation in IDH1 is less than the predetermined cutoff value, wherein the second treatment comprises at least one CD70-targeting agent.

The present disclosure also provides methods of identifying if a subject having AML will be responsive to an AML treatment comprising the administration of at least one CD70-targeting agent, the methods comprising: a) determining the mutational status of IDH1 in a plurality of cells in a sample from the subject; and b) identifying that the subject will be responsive to the AML treatment when no mutations in IDH1 are identified.

The present disclosure also provides methods of identifying if a subject having AML will be responsive to an AML treatment comprising the administration of at least one CD70-targeting agent, the methods comprising: a) determining the mutational status of IDH1 in a plurality of cells in a sample from the subject; b) determining the number and/or percentage of cells in the plurality of cells that exhibit at least one mutation in IDH1 based on step (a); c) comparing the number and/or percentage identified in step (b) to a predetermined cutoff value; and d) identifying that the subject will be responsive to the AML treatment when the number and/or percentage of cells that exhibit at least one mutation in IDH1 is less than the predetermined cutoff value.

The present disclosure provides methods of treating AML in a subject in need thereof, the methods comprising administering at least one CD70-targeting agent to the subject, wherein the subject no AML cells exhibit a mutation in IDH1. The present disclosure provides methods of treating AML in a subject in need thereof, the methods comprising administering at least one CD70-targeting agent to the subject, wherein the subject has a number and/or percentage of AML cells that exhibit a mutation in IDH1 that is less than a predetermined cutoff value.

In some aspects, the preceding methods can further comprise administering to the subject a combination of at least one BCL-2 inhibitor and at least one hypomethylating agent. For example, treatments that do not comprise CD-70 targeting agents can comprise at least one BCL-2 inhibitor and at least one hypomethylating agent.

### IDH2-Based Methods

The following methods are based, inter alia, on the discovery (presented herein in the experimental examples) that AML subjects having mutated IDH2 may respond favorably to treatments that do not comprise the administration of at least one CD70-targeting agent, and therefore said subjects can be excluded from treatment with a CD70-targeting agent.

Accordingly, the present disclosure provides methods of treating acute myeloid leukemia (AML) in a subject, the methods comprising: a) determining the mutational status of IDH2 in a plurality of cells in a sample from the subject; and b) administering to the subject a first treatment when at least one mutation in IDH2 are identified, wherein the first treatment does not comprise at least one CD70-targeting agent. In some aspects, the method can further comprise administering to the subject a second treatment when no mutations in IDH2 are identified, wherein the second treatment does comprises at least one CD70-targeting agent.

The present disclosure also provides methods of treating acute myeloid leukemia (AML) in a subject, the methods comprising: a) determining the mutational status of IDH2 in a plurality of cells in a sample from the subject; b) determining the number and/or percentage of cells in the plurality of cells that exhibit at least one mutation in IDH2 based on step (a); c) comparing the number and/or percentage identified in step (b) to a predetermined cutoff value; and d) administering to the subject a first treatment when the number and/or percentage of cells that exhibit at least one mutation in IDH2 is equal to or greater than the predetermined cutoff value, wherein the first treatment does not comprise at least one CD70-targeting agent. In some aspects, the method can further comprise administering to the subject a second treatment when the number and/or percentage of cells that exhibit at least one mutation in IDH2 is less than the predetermined cutoff value, wherein the second treatment comprises at least one CD70-targeting agent.

The present disclosure also provides methods of identifying if a subject having AML will be responsive to an AML treatment comprising the administration of at least one CD70-targeting agent, the methods comprising: a) determining the mutational status of IDH2 in a plurality of cells in a sample from the subject; and b) identifying that the subject will be responsive to the AML treatment when no mutations in IDH2 are identified.

The present disclosure also provides methods of identifying if a subject having AML will be responsive to an AML treatment comprising the administration of at least one CD70-targeting agent, the methods comprising: a) determining the mutational status of IDH2 in a plurality of cells in a sample from the subject; b) determining the number and/or percentage of cells in the plurality of cells that exhibit at least one mutation in IDH2 based on step (a); c) comparing the number and/or percentage identified in step (b) to a predetermined cutoff value; and d) identifying that the subject will be responsive to the AML treatment when the number and/or percentage of cells that exhibit at least one mutation in IDH2 is less than the predetermined cutoff value.

The present disclosure provides methods of treating AML in a subject in need thereof, the methods comprising administering at least one CD70-targeting agent to the subject, wherein the subject has no AML cells that exhibit a mutation in IDH2. The present disclosure provides methods of treating AML in a subject in need thereof, the methods comprising administering at least one CD70-targeting agent to the subject, wherein the subject has a number and/or percentage of AML cells that exhibit a mutation in IDH2 that is less than a predetermined cutoff value.

In some aspects, the preceding methods can further comprise administering to the subject a combination of at least one BCL-2 inhibitor and at least one hypomethylating agent. For example, treatments that do not comprise CD-70 targeting agents can comprise at least one BCL-2 inhibitor and at least one hypomethylating agent.

### PTPN11-Based Methods

The following methods are based, inter alia, on the discovery (presented herein in the experimental examples) that AML subjects having mutated PTPN11 may respond more favorably to treatment that comprises the administration of at least one CD70-targeting agent as opposed to a treatment that does not comprise the administration of at least one CD70-targeting agent.

Accordingly, the present disclosure provides methods of treating acute myeloid leukemia (AML) in a subject, the methods comprising: a) determining the mutational status of PTPN11 in a plurality of cells in a sample from the subject; and b) administering to the subject a first treatment when at least one mutations in PTPN11 are identified, wherein the first treatment comprises at least one CD70-targeting agent. In some aspects, the method can further comprise administering to the subject a second treatment when no mutations in PTPN11 are identified, wherein the second treatment does not comprise a CD70-targeting agent.

The present disclosure also provides methods of treating acute myeloid leukemia (AML) in a subject, the methods comprising: a) determining the mutational status of PTPN11 in a plurality of cells in a sample from the subject; b) determining the number and/or percentage of cells in the plurality of cells that exhibit at least one mutation in PTPN11 based on step (a); c) comparing the number and/or percentage identified in step (b) to a predetermined cutoff value; and d) administering to the subject a first treatment when the number and/or percentage of cells that exhibit at least one mutation in PTPN11 is equal to or greater than the predetermined cutoff value, wherein the first treatment comprises at least one CD70-targeting agent. In some aspects, the method can further comprise administering to the subject a second treatment when the number and/or percentage of cells that exhibit at least one mutation in PTPN11 is less than the predetermined cutoff value, wherein the second treatment does not comprise a CD70-targeting agent.

The present disclosure also provides methods of identifying if a subject having AML will be responsive to an AML treatment comprising the administration of at least one CD70-targeting agent, the methods comprising: a) determining the mutational status of PTPN11 in a plurality of cells in a sample from the subject; and b) identifying that the subject will be responsive to the AML treatment when the presence of at least one mutation in PTPN11 is identified.

The present disclosure also provides methods of identifying if a subject having AML will be responsive to an AML treatment comprising the administration of at least one CD70-targeting agent, the methods comprising: a) determining the mutational status of PTPN11 in a plurality of cells in a sample from the subject; b) determining the number and/or percentage of cells in the plurality of cells that exhibit at least one mutation in PTPN11 based on step (a); c) comparing the number and/or percentage identified in step (b) to a predetermined cutoff value; and d) identifying that the subject will be responsive to the AML treatment when the number and/or percentage of cells that exhibit at least one mutation in PTPN11 is equal to or greater than the predetermined cutoff value.

The present disclosure provides methods of treating AML in a subject in need thereof, the methods comprising administering at least one CD70-targeting agent to the subject, wherein the subject has at least one AML cell that exhibits a mutation in PTPN11. The present disclosure provides methods of treating AML in a subject in need thereof, the methods comprising administering at least one CD70-targeting agent to the subject, wherein the subject has a number and/or percentage of AML cells that exhibit a mutation in PTPN11 that is equal to or greater than a predetermined cutoff value.

In some aspects, the preceding methods can further comprise administering to the subject a combination of at least one BCL-2 inhibitor and at least one hypomethylating agent.

### BRAF-Based Methods

The following methods are based, inter alia, on the discovery (presented herein in the experimental examples) that AML subjects having mutated BRAF may respond more favorably to treatment that comprises the administration of at least one CD70-targeting agent as opposed to a treatment that does not comprise the administration of at least one CD70-targeting agent.

Accordingly, the present disclosure provides methods of treating acute myeloid leukemia (AML) in a subject, the methods comprising: a) determining the mutational status of BRAF in a plurality of cells in a sample from the subject; and b) administering to the subject a first treatment when at least one mutations in BRAF are identified, wherein the first treatment comprises at least one CD70-targeting agent. In some aspects, the method can further comprise administering to the subject a second treatment when no mutations in BRAF are identified, wherein the second treatment does not comprise a CD70-targeting agent.

The present disclosure also provides methods of treating acute myeloid leukemia (AML) in a subject, the methods comprising: a) determining the mutational status of BRAF in a plurality of cells in a sample from the subject; b) determining the number and/or percentage of cells in the plurality of cells that exhibit at least one mutation in BRAF based on step (a); c) comparing the number and/or percentage identified in step (b) to a predetermined cutoff value; and d) administering to the subject a first treatment when the number and/or percentage of cells that exhibit at least one mutation in BRAF is equal to or greater than the predetermined cutoff value, wherein the first treatment comprises at least one CD70-targeting agent. In some aspects, the method can further comprise administering to the subject a second treatment when the number and/or percentage of cells that exhibit at least one mutation in BRAF is less than the predetermined cutoff value, wherein the second treatment does not comprise a CD70-targeting agent.

The present disclosure also provides methods of identifying if a subject having AML will be responsive to an AML treatment comprising the administration of at least one CD70-targeting agent, the methods comprising: a) determining the mutational status of BRAF in a plurality of cells in a sample from the subject; and b) identifying that the subject will be responsive to the AML treatment when the presence of at least one mutation in BRAF is identified.

The present disclosure also provides methods of identifying if a subject having AML will be responsive to an AML treatment comprising the administration of at least one CD70-targeting agent, the methods comprising: a) determining the mutational status of BRAF in a plurality of cells in a sample from the subject; b) determining the number and/or percentage of cells in the plurality of cells that exhibit at least one mutation in BRAF based on step (a); c) comparing the number and/or percentage identified in step (b) to a predetermined cutoff value, and d) identifying that the subject will be responsive to the AML treatment when the number and/or percentage of cells that exhibit at least one mutation in BRAF is equal to or greater than the predetermined cutoff value.

The present disclosure provides methods of treating AML in a subject in need thereof, the methods comprising administering at least one CD70-targeting agent to the subject, wherein the subject has at least one AML cell that exhibits a mutation in BRAF. The present disclosure provides methods of treating AML in a subject in need thereof, the methods comprising administering at least one CD70-targeting agent to the subject, wherein the subject has a number and/or percentage of AML cells that exhibit a mutation in BRAF that is equal to or greater than a predetermined cutoff value.

In some aspects, the preceding methods can further comprise administering to the subject a combination of at least one BCL-2 inhibitor and at least one hypomethylating agent.

### JAK2-Based Methods

The following methods are based, inter alia, on the discovery (presented herein in the experimental examples) that AML subjects having mutated JAK2 may respond more favorably to treatment that comprises the administration of at least one CD70-targeting agent as opposed to a treatment that does not comprise the administration of at least one CD70-targeting agent.

Accordingly, the present disclosure provides methods of treating acute myeloid leukemia (AML) in a subject, the methods comprising: a) determining the mutational status of JAK2 in a plurality of cells in a sample from the subject; and b) administering to the subject a first treatment when at least one mutations in JAK2 are identified, wherein the first treatment comprises at least one CD70-targeting agent. In some aspects, the method can further comprise administering to the subject a second treatment when no mutations in JAK2 are identified, wherein the second treatment does not comprise a CD70-targeting agent.

The present disclosure also provides methods of treating acute myeloid leukemia (AML) in a subject, the methods comprising: a) determining the mutational status of JAK2 in a plurality of cells in a sample from the subject; b) determining the number and/or percentage of cells in the plurality of cells that exhibit at least one mutation in JAK2 based on step (a); c) comparing the number and/or percentage identified in step (b) to a predetermined cutoff value; and d) administering to the subject a first treatment when the number and/or percentage of cells that exhibit at least one mutation in JAK2 is equal to or greater than the predetermined cutoff value, wherein the first treatment comprises at least one CD70-targeting agent. In some aspects, the method can further comprise administering to the subject a second treatment when the number and/or percentage of cells that exhibit at least one mutation in JAK2 is less than the predetermined cutoff value, wherein the second treatment does not comprise a CD70-targeting agent.

The present disclosure also provides methods of identifying if a subject having AML will be responsive to an AML treatment comprising the administration of at least one CD70-targeting agent, the methods comprising: a) determining the mutational status of JAK2 in a plurality of cells in a sample from the subject; and b) identifying that the subject will be responsive to the AML treatment when the presence of at least one mutation in JAK2 is identified.

The present disclosure also provides methods of identifying if a subject having AML will be responsive to an AML treatment comprising the administration of at least one CD70-targeting agent, the methods comprising: a) determining the mutational status of JAK2 in a plurality of cells in a sample from the subject; b) determining the number and/or percentage of cells in the plurality of cells that exhibit at least one mutation in JAK2 based on step (a); c) comparing the number and/or percentage identified in step (b) to a predetermined cutoff value; and d) identifying that the subject will be responsive to the AML treatment when the number and/or percentage of cells that exhibit at least one mutation in JAK2 is equal to or greater than the predetermined cutoff value.

The present disclosure provides methods of treating AML in a subject in need thereof, the methods comprising administering at least one CD70-targeting agent to the subject, wherein the subject has at least one AML cell that exhibits a mutation in JAK2. The present disclosure provides methods of treating AML in a subject in need thereof, the methods comprising administering at least one CD70-targeting agent to the subject, wherein the subject has a number and/or percentage of AML cells that exhibit a mutation in JAK2 that is equal to or greater than a predetermined cutoff value.

In some aspects, the preceding methods can further comprise administering to the subject a combination of at least one BCL-2 inhibitor and at least one hypomethylating agent.

### Monocytic AML-Based Methods

The following methods are based, inter alia, on the discovery that AML subjects having monocytic subclones may respond more favorably to treatment that comprises the administration of at least one CD70-targeting agent as opposed to a treatment that does not comprise the administration of at least one CD70-targeting agent.

Accordingly, the present disclosure provides methods of treating acute myeloid leukemia (AML) in a subject, the methods comprising: a) determining if the subject has monocytic AML; and b) administering to the subject a first treatment when the subject is identified as having monocytic AML, wherein the first treatment comprises at least one CD70-targeting agent. In some aspects, the method further comprises administering to the subject a second treatment when the subject is identified as not having monocytic AML, wherein the second treatment does not comprise a CD70-targeting agent.

The present disclosure also provides methods of identifying if a subject having AML will be responsive to an AML treatment comprising the administration of at least one CD70-targeting agent, the methods comprising: a) determining if the subject has monocytic AML; b) identifying that the subject will be responsive to the AML treatment when the subject is identified as having monocytic AML.

The present disclosure provides methods of treating AML in a subject in need thereof, the methods comprising administering at least one CD70-targeting agent to the subject, wherein the subject has monocytic AML. In some aspects, the preceding methods can further comprise administering to the subject a combination of at least one BCL-2 inhibitor and at least one hypomethylating agent.

In some aspects, determining if a subject has monocytic AML can comprise any standard method known in the art for identifying monocytic AML. In some aspects, determining if a subject has monocytic AML can comprise determining if the subject has French-American-British (FAB) M5 AML (see Vardiman J W, Harris N L, Brunning R D. The WHO classification of myeloid neoplasms. Blood 2002; 100:2292, which is incorporated herein in its entirety for all purposes).

In some aspects, identifying if a subject has monocytic AML can comprise measuring the expression of at least one of CD11b, CD14, CD36, CD64, CD93 and CLL-1 in a plurality of cells in a sample from the subject; and b) identifying that the subject has monocytic AML when at least about 10% of the cells from the subject are at least one of CD11b+, CD14+, CD36+, CD64+, CD93+ and CLL-1+. In a non-limiting example, identifying if a subject has monocytic AML can comprise measuring the expression of each of CD11b, CD14, CD36, CD64, CD93 and CLL-1 in a plurality of cells in a sample from the subject; and b) identifying that the subject has monocytic AML when at least about 10% of the cells from the subject are CD11b+, CD14+, CD36+, CD64+. CD93+ and CLL-1+.

### BCOR-Based Methods

The following methods are based, inter alia, on the discovery (presented herein in the experimental examples) that AML subjects having mutated BCOR may respond favorably to treatments that do not comprise the administration of at least one CD70-targeting agent, and therefore said subjects can be excluded from treatment with a CD70-targeting agent.

Accordingly, the present disclosure provides methods of treating acute myeloid leukemia (AML) in a subject, the methods comprising: a) determining the mutational status of BCOR in a plurality of cells in a sample from the subject; and b) administering to the subject a first treatment when at least one mutation in BCOR are identified, wherein the first treatment does not comprise at least one CD70-targeting agent. In some aspects, the method can further comprise administering to the subject a second treatment when no mutations in BCOR are identified, wherein the second treatment comprises at least one CD70-targeting agent.

The present disclosure also provides methods of treating acute myeloid leukemia (AML) in a subject, the methods comprising: a) determining the mutational status of BCOR in a plurality of cells in a sample from the subject; b) determining the number and/or percentage of cells in the plurality of cells that exhibit at least one mutation in BCOR based on step (a); c) comparing the number and/or percentage identified in step (b) to a predetermined cutoff value; and d) administering to the subject a first treatment when the number and/or percentage of cells that exhibit at least one mutation in BCOR is equal to or greater than the predetermined cutoff value, wherein the first treatment does not comprise at least one CD70-targeting agent. In some aspects, the method further comprises administering to the subject a second treatment when the number and/or percentage of cells that exhibit at least one mutation in BCOR is less than the predetermined cutoff value, wherein the second treatment comprises at least one CD70-targeting agent.

The present disclosure also provides methods of identifying if a subject having AML will be responsive to an AML treatment comprising the administration of at least one CD70-targeting agent, the methods comprising: a) determining the mutational status of BCOR in a plurality of cells in a sample from the subject; and b) identifying that the subject will be responsive to the AML treatment when no mutations in BCOR are identified.

The present disclosure also provides methods of identifying if a subject having AML will be responsive to an AML treatment comprising the administration of at least one CD70-targeting agent, the methods comprising: a) determining the mutational status of BCOR in a plurality of cells in a sample from the subject; b) determining the number and/or percentage of cells in the plurality of cells that exhibit at least one mutation in BCOR based on step (a); c) comparing the number and/or percentage identified in step (b) to a predetermined cutoff value; and d) identifying that the subject will be responsive to the AML treatment when the number and/or percentage of cells that exhibit at least one mutation in BCOR is less than the predetermined cutoff value.

The present disclosure provides methods of treating AML in a subject in need thereof, the methods comprising administering at least one CD70-targeting agent to the subject, wherein the subject has no AML cells that exhibit a mutation in BCOR The present disclosure provides methods of treating AML in a subject in need thereof, the methods comprising administering at least one CD70-targeting agent to the subject, wherein the subject has a number and/or percentage of AML cells that exhibit a mutation in BCOR that is less than a predetermined cutoff value.

In some aspects, the preceding methods can further comprise administering to the subject a combination of at least one BCL-2 inhibitor and at least one hypomethylating agent. For example, treatments that do not comprise CD-70 targeting agents can comprise at least one BCL-2 inhibitor and at least one hypomethylating agent.

### BCORL1-Based Methods

The following methods are based, inter alia, on the discovery (presented herein in the experimental examples) that AML subjects having mutated BCORL1 may respond favorably to treatments that do not comprise the administration of at least one CD70-targeting agent, and therefore said subjects can be excluded from treatment with a CD70-targeting agent.

Accordingly, the present disclosure provides methods of treating acute myeloid leukemia (AML) in a subject, the methods comprising: a) determining the mutational status of BCORL1 in a plurality of cells in a sample from the subject; and b) administering to the subject a first treatment when at least one mutation in BCORL1 are identified, wherein the first treatment does not comprise at least one CD70-targeting agent. In some aspects, the method can further comprise administering to the subject a second treatment when no mutations in BCORL1 are identified, wherein the second treatment comprises at least one CD70-targeting agent.

The present disclosure also provides methods of treating acute myeloid leukemia (AML) in a subject, the methods comprising: a) determining the mutational status of BCORL1 in a plurality of cells in a sample from the subject; b) determining the number and/or percentage of cells in the plurality of cells that exhibit at least one mutation in BCORL1 based on step (a); c) comparing the number and/or percentage identified in step (b) to a predetermined cutoff value; and d) administering to the subject a first treatment when the number and/or percentage of cells that exhibit at least one mutation in BCORL1 is equal to or greater than the predetermined cutoff value, wherein the first treatment does not comprise at least one CD70-targeting agent. In some aspects, the method further comprises administering to the subject a second treatment when the number and/or percentage of cells that exhibit at least one mutation in BCORL1 is less than the predetermined cutoff value, wherein the second treatment comprises at least one CD70-targeting agent.

The present disclosure also provides methods of identifying if a subject having AML will be responsive to an AML treatment comprising the administration of at least one CD70-targeting agent, the methods comprising: a) determining the mutational status of BCORL1 in a plurality of cells in a sample from the subject; and b) identifying that the subject will be responsive to the AML treatment when no mutations in BCORL1 are identified.

The present disclosure also provides methods of identifying if a subject having AML will be responsive to an AML treatment comprising the administration of at least one CD70-targeting agent, the methods comprising: a) determining the mutational status of BCORL1 in a plurality of cells in a sample from the subject; b) determining the number and/or percentage of cells in the plurality of cells that exhibit at least one mutation in BCORL1 based on step (a); c) comparing the number and/or percentage identified in step (b) to a predetermined cutoff value; and d) identifying that the subject will be responsive to the AML treatment when the number and/or percentage of cells that exhibit at least one mutation in BCORL1 is less than the predetermined cutoff value.

The present disclosure provides methods of treating AML in a subject in need thereof, the methods comprising administering at least one CD70-targeting agent to the subject, wherein the subject has no AML cells that exhibit a mutation in BCORL1. The present disclosure provides methods of treating AML in a subject in need thereof, the methods comprising administering at least one CD70-targeting agent to the subject, wherein the subject has a number and/or percentage of AML cells that exhibit a mutation in BCORL1 that is less than a predetermined cutoff value.

In some aspects, the preceding methods can further comprise administering to the subject a combination of at least one BCL-2 inhibitor and at least one hypomethylating agent. For example, treatments that do not comprise CD-70 targeting agents can comprise at least one BCL-2 inhibitor and at least one hypomethylating agent.

### EZH2-Based Methods

The following methods are based, inter alia, on the discovery (presented herein in the experimental examples) that AML subjects having mutated EZH2 may respond favorably to treatment that does not comprise the administration of at least one CD70-targeting agent, and therefore said subjects can be excluded from treatment with a CD70-targeting agent.

Accordingly, the present disclosure provides methods of treating acute myeloid leukemia (AML) in a subject, the methods comprising: a) determining the mutational status of EZH2 in a plurality of cells in a sample from the subject; and b) administering to the subject a first treatment when at least one mutation in EZH2 are identified, wherein the first treatment does not comprise at least one CD70-targeting agent. In some aspects, the method can further comprise administering to the subject a second treatment when no mutations in EZH2 are identified, wherein the second treatment comprises at least one a CD70-targeting agent.

The present disclosure also provides methods of treating acute myeloid leukemia (AML) in a subject, the methods comprising: a) determining the mutational status of EZH2 in a plurality of cells in a sample from the subject; b) determining the number and/or percentage of cells in the plurality of cells that exhibit at least one mutation in EZ12 based on step (a); c) comparing the number and/or percentage identified in step (b) to a predetermined cutoff value; and d) administering to the subject a first treatment when the number and/or percentage of cells that exhibit at least one mutation in EZH2 is equal to or greater than the predetermined cutoff value, wherein the first treatment does not comprise at least one CD70-targeting agent. In some aspects, the method further comprises administering to the subject a second treatment when the number and/or percentage of cells that exhibit at least one mutation in EZH2 is less than the predetermined cutoff value, wherein the second treatment comprises at least one CD70-targeting agent.

The present disclosure also provides methods of identifying if a subject having AML will be responsive to an AML treatment comprising the administration of at least one CD70-targeting agent, the methods comprising: a) determining the mutational status of EZH2 in a plurality of cells in a sample from the subject; and b) identifying that the subject will be responsive to the AML treatment when no mutations in EZH2 are identified.

The present disclosure also provides methods of identifying if a subject having AML will be responsive to an AML treatment comprising the administration of at least one CD70-targeting agent, the methods comprising: a) determining the mutational status of EZH2 in a plurality of cells in a sample from the subject; b) determining the number and/or percentage of cells in the plurality of cells that exhibit at least one mutation in EZH2 based on step (a); c) comparing the number and/or percentage identified in step (b) to a predetermined cutoff value; and d) identifying that the subject will be responsive to the AML treatment when the number and/or percentage of cells that exhibit at least one mutation in EZH2 is less than the predetermined cutoff value.

The present disclosure provides methods of treating AML in a subject in need thereof, the methods comprising administering at least one CD70-targeting agent to the subject, wherein the subject has no AML cells that exhibit a mutation in EZH2. The present disclosure provides methods of treating AML in a subject in need thereof, the methods comprising administering at least one CD70-targeting agent to the subject, wherein the subject has a number and/or percentage of AML cells that exhibit a mutation in EZH2 that is less than a predetermined cutoff value.

In some aspects, the preceding methods can further comprise administering to the subject a combination of at least one BCL-2 inhibitor and at least one hypomethylating agent. For example, treatments that do not comprise CD-70 targeting agents can comprise at least one BCL-2 inhibitor and at least one hypomethylating agent.

### FLT3-ITD-Based Methods

The following methods are based, inter alia, on the discovery (presented herein in the experimental examples) that AML subjects having FLT3-ITD may respond more favorably to treatment that comprises the administration of at least one CD70-targeting agent as opposed to a treatment that does not comprise the administration of at least one CD70-targeting agent.

Accordingly, the present disclosure provides methods of treating acute myeloid leukemia (AML) in a subject, the methods comprising: a) determining the mutational status of FLT3 in a plurality of cells in a sample from the subject; and b) administering to the subject a first treatment when at least one FLT3-ITD mutation is identified, wherein the first treatment comprises at least one CD70-targeting agent. In some aspects, the method can further comprise administering to the subject a second treatment when no FLT3-ITD mutations are identified, wherein the second treatment does not comprise a CD70-targeting agent.

The present disclosure also provides methods of treating acute myeloid leukemia (AML) in a subject, the methods comprising: a) determining the mutational status of FLT3 in a plurality of cells in a sample from the subject; b) determining the number and/or percentage of cells in the plurality of cells that exhibit the FLT3-ITD mutation based on step (a); c) comparing the number and/or percentage identified in step (b) to a predetermined cutoff value; and d) administering to the subject a first treatment when the number and/or percentage of cells that exhibit the FLT3-ITD mutation is equal to or greater than the predetermined cutoff value, wherein the first treatment comprises at least one CD70-targeting agent. In some aspects, the method further comprises administering to the subject a second treatment when the number and/or percentage of cells that exhibit the FLT3-ITD mutation is less than the predetermined cutoff value, wherein the second treatment does not comprise a CD70-targeting agent.

The present disclosure also provides methods of identifying if a subject having AML will be responsive to an AML treatment comprising the administration of at least one CD70-targeting agent, the methods comprising: a) determining the mutational status of FLT3 in a plurality of cells in a sample from the subject; and b) identifying that the subject will be responsive to the AML treatment when the presence of the FLT3-ITD mutation is identified.

The present disclosure also provides methods of identifying if a subject having AML will be responsive to an AML treatment comprising the administration of at least one CD70-targeting agent, the methods comprising: a) determining the mutational status of FLT3 in a plurality of cells in a sample from the subject; b) determining the number and/or percentage of cells in the plurality of cells that exhibit the FLT3-ITD mutation based on step (a); c) comparing the number and/or percentage identified in step (b) to a predetermined cutoff value; and d) identifying that the subject will be responsive to the AML treatment when the number and/or percentage of cells that exhibit the FLT3-ITD mutation is equal to or greater than the predetermined cutoff value.

The present disclosure provides methods of treating AML in a subject in need thereof, the methods comprising administering at least one CD70-targeting agent to the subject, wherein the subject has at least one AML cell that exhibits the FLT3-ITD mutation. The present disclosure provides methods of treating AML in a subject in need thereof, the methods comprising administering at least one CD70-targeting agent to the subject, wherein the subject has a number and/or percentage of AML cells that exhibit the FLT3-ITD mutation is equal to or greater than a predetermined cutoff value. In some aspects, the preceding methods can further comprise administering to the subject a combination of at least one BCL-2 inhibitor and at least one hypomethylating agent.

In some aspects, a FLT3 mutation can be a FLT3-ITD mutation. In some aspects, a FLT3 mutation is a mutation other than FLT3-ITD.

### KRAS-Based Methods

The following methods are based, inter alia, on the discovery (presented herein in the experimental examples) that AML subjects having mutated KRAS may respond more favorably to treatment that comprises the administration of at least one CD70-targeting agent as opposed to a treatment that does not comprise the administration of at least one CD70-targeting agent.

Accordingly, the present disclosure provides methods of treating acute myeloid leukemia (AML) in a subject, the methods comprising: a) determining the mutational status of KRAS in a plurality of cells in a sample from the subject; and b) administering to the subject a first treatment when at least one mutation in KRAS are identified, wherein the first treatment comprises at least one CD70-targeting agent. In some aspects, the method can further comprise administering to the subject a second treatment when no mutations in KRAS are identified, wherein the second treatment does not comprise a CD70-targeting agent.

The present disclosure also provides methods of treating acute myeloid leukemia (AML) in a subject, the methods comprising: a) determining the mutational status of KRAS in a plurality of cells in a sample from the subject; b) determining the number and/or percentage of cells in the plurality of cells that exhibit at least one mutation in KRAS based on step (a); c) comparing the number and/or percentage identified in step (b) to a predetermined cutoff value; and d) administering to the subject a first treatment when the number and/or percentage of cells that exhibit at least one mutation in KRAS is equal to or greater than the predetermined cutoff value, wherein the first treatment comprises at least one CD70-targeting agent. In some aspects, the method further comprises administering to the subject a second treatment when the number and/or percentage of cells that exhibit at least one mutation in KRAS is less than the predetermined cutoff value, wherein the second treatment does not comprise a CD70-targeting agent.

The present disclosure also provides methods of identifying if a subject having AML will be responsive to an AML treatment comprising the administration of at least one CD70-targeting agent, the methods comprising: a) determining the mutational status of KRAS in a plurality of cells in a sample from the subject; and b) identifying that the subject will be responsive to the AML treatment when the presence of at least one mutation in KRAS is identified.

The present disclosure also provides methods of identifying if a subject having AML will be responsive to an AML treatment comprising the administration of at least one CD70-targeting agent, the methods comprising: a) determining the mutational status of NRAS in a plurality of cells in a sample from the subject; b) determining the number and/or percentage of cells in the plurality of cells that exhibit at least one mutation in KRAS based on step (a); c) comparing the number and/or percentage identified in step (b) to a predetermined cutoff value; and d) identifying that the subject will be responsive to the AML treatment when the number and/or percentage of cells that exhibit at least one mutation in KRAS is equal to or greater than the predetermined cutoff value.

The present disclosure provides methods of treating AML in a subject in need thereof, the methods comprising administering at least one CD70-targeting agent to the subject, wherein the subject has at least one AML cell that exhibits a mutation in KRAS. The present disclosure provides methods of treating AML in a subject in need thereof, the methods comprising administering at least one CD70-targeting agent to the subject, wherein the subject has a number and/or percentage of AML cells that exhibit a mutation in KRAS that is equal to or greater than a predetermined cutoff value. In some aspects, the preceding methods can further comprise administering to the subject a combination of at least one BCL-2 inhibitor and at least one hypomethylating agent.

### RUNX1-Based Methods

The following methods are based, inter alia, on the discovery (presented herein in the experimental examples) that AML subjects having mutated RUNX1 may respond favorably to treatments that do not comprise the administration of at least one CD70-targeting agent, and therefore said subjects can be excluded from treatment with a CD70-targeting agent.

Accordingly, the present disclosure provides methods of treating acute myeloid leukemia (AML) in a subject, the methods comprising: a) determining the mutational status of RUNX1 in a plurality of cells in a sample from the subject; and b) administering to the subject a first treatment when at least one mutation in RUNX1 are identified, wherein the first treatment does not comprise at least one CD70-targeting agent. In some aspects, the method can further comprise administering to the subject a second treatment when no mutations in RUNX1 are identified, wherein the second treatment comprises at least one CD70-targeting agent.

The present disclosure also provides methods of treating acute myeloid leukemia (AML) in a subject, the methods comprising: a) determining the mutational status of RUNX1 in a plurality of cells in a sample from the subject; b) determining the number and/or percentage of cells in the plurality of cells that exhibit at least one mutation in RUNX1 based on step (a); c) comparing the number and/or percentage identified in step (b) to a predetermined cutoff value; and d) administering to the subject a first treatment when the number and/or percentage of cells that exhibit at least one mutation in RUNX1 is equal to or greater than the predetermined cutoff value, wherein the first treatment does not comprise at least one CD70-targeting agent. In some aspects, the method further comprises administering to the subject a second treatment when the number and/or percentage of cells that exhibit at least one mutation in RUNX1 is less than the predetermined cutoff value, wherein the second treatment comprises at least one CD70-targeting agent.

The present disclosure also provides methods of identifying if a subject having AML will be responsive to an AML treatment comprising the administration of at least one CD70-targeting agent, the methods comprising: a) determining the mutational status of RUNX1 in a plurality of cells in a sample from the subject; and b) identifying that the subject will be responsive to the AML treatment when no mutations in RUNX1 are identified.

The present disclosure also provides methods of identifying if a subject having AML will be responsive to an AML treatment comprising the administration of at least one CD70-targeting agent, the methods comprising: a) determining the mutational status of RUNX1 in a plurality of cells in a sample from the subject; b) determining the number and/or percentage of cells in the plurality of cells that exhibit at least one mutation in RUNX1 based on step (a); c) comparing the number and/or percentage identified in step (b) to a predetermined cutoff value; and d) identifying that the subject will be responsive to the AML treatment when the number and/or percentage of cells that exhibit at least one mutation in RUNX1 is less than the predetermined cutoff value.

The present disclosure provides methods of treating AML in a subject in need thereof, the methods comprising administering at least one CD70-targeting agent to the subject, wherein the subject has no AML cells that exhibit a mutation in RUNX1. The present disclosure provides methods of treating AML in a subject in need thereof, the methods comprising administering at least one CD70-targeting agent to the subject, wherein the subject has a number and/or percentage of AML cells that exhibit a mutation in RUNX1 that is less than a predetermined cutoff value.

In some aspects, the preceding methods can further comprise administering to the subject a combination of at least one BCL-2 inhibitor and at least one hypomethylating agent.

For example, treatments that do not comprise CD-70 targeting agents can comprise at least one BCL-2 inhibitor and at least one hypomethylating agent.

### SF3B1-Based Methods

The following methods are based, inter alia, on the discovery (presented herein in the experimental examples) that AML subjects having mutated SF3B1 may respond favorably to treatments that do not comprise the administration of at least one CD70-targeting agent, and therefore said subjects can be excluded from treatment with a CD70-targeting agent.

Accordingly, the present disclosure provides methods of treating acute myeloid leukemia (AML) in a subject, the methods comprising: a) determining the mutational status of SF3B1 in a plurality of cells in a sample from the subject; and b) administering to the subject a first treatment when at least one mutation in SF3B1 are identified, wherein the first treatment does not comprise at least one CD70-targeting agent. In some aspects, the method can further comprise administering to the subject a second treatment when no mutations in SF3B1 are identified, wherein the second treatment comprises at least one CD70-targeting agent.

The present disclosure also provides methods of treating acute myeloid leukemia (AML) in a subject, the methods comprising: a) determining the mutational status of SF3B1 in a plurality of cells in a sample from the subject; b) determining the number and/or percentage of cells in the plurality of cells that exhibit at least one mutation in SF3B1 based on step (a); c) comparing the number and/or percentage identified in step (b) to a predetermined cutoff value; and d) administering to the subject a first treatment when the number and/or percentage of cells that exhibit at least one mutation in SF3B1 is equal to or greater than the predetermined cutoff value, wherein the first treatment does not comprise at least one CD70-targeting agent. In some aspects, the method further comprises administering to the subject a second treatment when the number and/or percentage of cells that exhibit at least one mutation in SF3B1 is less than the predetermined cutoff value, wherein the second treatment comprises at least one CD70-targeting agent.

The present disclosure also provides methods of identifying if a subject having AML will be responsive to an AML treatment comprising the administration of at least one CD70-targeting agent, the methods comprising: a) determining the mutational status of SF3B1 in a plurality of cells in a sample from the subject; and b) identifying that the subject will be responsive to the AML treatment when no mutations in SF3B1 are identified.

The present disclosure also provides methods of identifying if a subject having AML will be responsive to an AML treatment comprising the administration of at least one CD70-targeting agent, the methods comprising: a) determining the mutational status of SF3B1 in a plurality of cells in a sample from the subject; b) determining the number and/or percentage of cells in the plurality of cells that exhibit at least one mutation in SF3B1 based on step (a); c) comparing the number and/or percentage identified in step (b) to a predetermined cutoff value; and d) identifying that the subject will be responsive to the AML treatment when the number and/or percentage of cells that exhibit at least one mutation in SF3B1 is less than the predetermined cutoff value.

The present disclosure provides methods of treating AML in a subject in need thereof, the methods comprising administering at least one CD70-targeting agent to the subject, wherein the subject has no AML cells that exhibit a mutation in SF3B1. The present disclosure provides methods of treating AML in a subject in need thereof, the methods comprising administering at least one CD70-targeting agent to the subject, wherein the subject has a number and/or percentage of AML cells that exhibit a mutation in SF3B1 that is less than a predetermined cutoff value.

In some aspects, the preceding methods can further comprise administering to the subject a combination of at least one BCL-2 inhibitor and at least one hypomethylating agent.

For example, treatments that do not comprise CD-70 targeting agents can comprise at least one BCL-2 inhibitor and at least one hypomethylating agent.

### SMC1A-Based Methods

The following methods are based, inter alia, on the discovery (presented herein in the experimental examples) that AML subjects having mutated SMC1A may respond more favorably to treatment that comprises the administration of at least one CD70-targeting agent as opposed to a treatment that does not comprise the administration of at least one CD70-targeting agent.

Accordingly, the present disclosure provides methods of treating acute myeloid leukemia (AML) in a subject, the methods comprising: a) determining the mutational status of SMC1A in a plurality of cells in a sample from the subject; and b) administering to the subject a first treatment when at least one mutation in SMC1A are identified, wherein the first treatment comprises at least one CD70-targeting agent. In some aspects, the method can further comprise administering to the subject a second treatment when no mutations in SMC1A are identified, wherein the second treatment does not comprise a CD70-targeting agent.

The present disclosure also provides methods of treating acute myeloid leukemia (AML) in a subject, the methods comprising: a) determining the mutational status of SMC1A in a plurality of cells in a sample from the subject; b) determining the number and/or percentage of cells in the plurality of cells that exhibit at least one mutation in SMC1A based on step (a); c) comparing the number and/or percentage identified in step (b) to a predetermined cutoff value; and d) administering to the subject a first treatment when the number and/or percentage of cells that exhibit at least one mutation in SMC1A is equal to or greater than the predetermined cutoff value, wherein the first treatment comprises at least one CD70-targeting agent. In some aspects, the method further comprises administering to the subject a second treatment when the number and/or percentage of cells that exhibit at least one mutation in SMC1A is less than the predetermined cutoff value, wherein the second treatment does not comprise a CD70-targeting agent.

The present disclosure also provides methods of identifying if a subject having AML will be responsive to an AML treatment comprising the administration of at least one CD70-targeting agent, the methods comprising: a) determining the mutational status of SMC1A in a plurality of cells in a sample from the subject; and b) identifying that the subject will be responsive to the AML treatment when the presence of at least one mutation in SMC1A is identified.

The present disclosure also provides methods of identifying if a subject having AML will be responsive to an AML treatment comprising the administration of at least one CD70-targeting agent, the methods comprising: a) determining the mutational status of SMC1A in a plurality of cells in a sample from the subject; b) determining the number and/or percentage of cells in the plurality of cells that exhibit at least one mutation in SMC1A based on step (a); c) comparing the number and/or percentage identified in step (b) to a predetermined cutoff value; and d) identifying that the subject will be responsive to the AML treatment when the number and/or percentage of cells that exhibit at least one mutation in SMC1A is equal to or greater than the predetermined cutoff value.

The present disclosure provides methods of treating AML in a subject in need thereof, the methods comprising administering at least one CD70-targeting agent to the subject, wherein the subject has at least one AML cell that exhibits a mutation in SMC1A. The present disclosure provides methods of treating AML in a subject in need thereof, the methods comprising administering at least one CD70-targeting agent to the subject, wherein the subject has a number and/or percentage of AML cells that exhibit a mutation in SMC1A that is equal to or greater than a predetermined cutoff value.

In some aspects, the preceding methods can further comprise administering to the subject a combination of at least one BCL-2 inhibitor and at least one hypomethylating agent.

### STAG2-Based Methods

The following methods are based, inter alia, on the discovery (presented herein in the experimental examples) that AML subjects having mutated STAG2 may respond more favorably to treatment that comprises the administration of at least one CD70-targeting agent as opposed to a treatment that does not comprise the administration of at least one CD70-targeting agent.

Accordingly, the present disclosure provides methods of treating acute myeloid leukemia (AML) in a subject, the methods comprising: a) determining the mutational status of STAG2 in a plurality of cells in a sample from the subject; and b) administering to the subject a first treatment when at least one mutation in STAG2 are identified, wherein the first treatment comprises at least one CD70-targeting agent. In some aspects, the method can further comprise administering to the subject a second treatment when no mutations in STAG2 are identified, wherein the second treatment does not comprise a CD70-targeting agent.

The present disclosure also provides methods of treating acute myeloid leukemia (AML) in a subject, the methods comprising: a) determining the mutational status of STAG2 in a plurality of cells in a sample from the subject; b) determining the number and/or percentage of cells in the plurality of cells that exhibit at least one mutation in STAG2 based on step (a); c) comparing the number and/or percentage identified in step (b) to a predetermined cutoff value; and d) administering to the subject a first treatment when the number and/or percentage of cells that exhibit at least one mutation in STAG2 is equal to or greater than the predetermined cutoff value, wherein the first treatment comprises at least one CD70-targeting agent. In some aspects, the method further comprises administering to the subject a second treatment when the number and/or percentage of cells that exhibit at least one mutation in STAG2 is less than the predetermined cutoff value, wherein the second treatment does not comprise a CD70-targeting agent.

The present disclosure also provides methods of identifying if a subject having AML will be responsive to an AML treatment comprising the administration of at least one CD70-targeting agent, the methods comprising: a) determining the mutational status of STAG2 in a plurality of cells in a sample from the subject; and b) identifying that the subject will be responsive to the AML treatment when the presence of at least one mutation in STAG2 is identified.

The present disclosure also provides methods of identifying if a subject having AML will be responsive to an AML treatment comprising the administration of at least one CD70-targeting agent, the methods comprising: a) determining the mutational status of STAG2 in a plurality of cells in a sample from the subject; b) determining the number and/or percentage of cells in the plurality of cells that exhibit at least one mutation in STAG2 based on step (a); c) comparing the number and/or percentage identified in step (b) to a predetermined cutoff value; and d) identifying that the subject will be responsive to the AML treatment when the number and/or percentage of cells that exhibit at least one mutation in STAG2 is equal to or greater than the predetermined cutoff value.

The present disclosure provides methods of treating AML in a subject in need thereof, the methods comprising administering at least one CD70-targeting agent to the subject, wherein the subject has at least one AML cell that exhibits a mutation in STAG2. The present disclosure provides methods of treating AML in a subject in need thereof, the methods comprising administering at least one CD70-targeting agent to the subject, wherein the subject has a number and/or percentage of AML cells that exhibit a mutation in STAG2 that is equal to or greater than a predetermined cutoff value.

In some aspects, the preceding methods can further comprise administering to the subject a combination of at least one BCL-2 inhibitor and at least one hypomethylating agent.

### TET2-Based Methods

The following methods are based, inter alia, on the discovery (presented herein in the experimental examples) that AML subjects having mutated TET2 may respond more favorably to treatment that comprises the administration of at least one CD70-targeting agent as opposed to a treatment that does not comprise the administration of at least one CD70-targeting agent.

Accordingly, the present disclosure provides methods of treating acute myeloid leukemia (AML) in a subject, the methods comprising: a) determining the mutational status of TET2 in a plurality of cells in a sample from the subject; and b) administering to the subject a first treatment when at least one mutation in TET2 are identified, wherein the first treatment comprises at least one CD70-targeting agent. In some aspects, the method can further comprise administering to the subject a second treatment when no mutations in TET2 are identified, wherein the second treatment does not comprise a CD70-targeting agent.

The present disclosure also provides methods of treating acute myeloid leukemia (AML) in a subject, the methods comprising: a) determining the mutational status of TET2 in a plurality of cells in a sample from the subject; b) determining the number and/or percentage of cells in the plurality of cells that exhibit at least one mutation in TET2 based on step (a); c) comparing the number and/or percentage identified in step (b) to a predetermined cutoff value; and d) administering to the subject a first treatment when the number and/or percentage of cells that exhibit at least one mutation in TET2 is equal to or greater than the predetermined cutoff value, wherein the first treatment comprises at least one CD70-targeting agent. In some aspects, the method further comprises administering to the subject a second treatment when the number and/or percentage of cells that exhibit at least one mutation in TET2 is less than the predetermined cutoff value, wherein the second treatment does not comprise a CD70-targeting agent.

The present disclosure also provides methods of identifying if a subject having AML will be responsive to an AML treatment comprising the administration of at least one CD70-targeting agent, the methods comprising: a) determining the mutational status of TET2 in a plurality of cells in a sample from the subject; and b) identifying that the subject will be responsive to the AML treatment when the presence of at least one mutation in TET2 is identified.

The present disclosure also provides methods of identifying if a subject having AML will be responsive to an AML treatment comprising the administration of at least one CD70-targeting agent, the methods comprising: a) determining the mutational status of TET2 in a plurality of cells in a sample from the subject; b) determining the number and/or percentage of cells in the plurality of cells that exhibit at least one mutation in TET2 based on step (a); c) comparing the number and/or percentage identified in step (b) to a predetermined cutoff value; and d) identifying that the subject will be responsive to the AML treatment when the number and/or percentage of cells that exhibit at least one mutation in TET2 is equal to or greater than the predetermined cutoff value.

The present disclosure provides methods of treating AML in a subject in need thereof, the methods comprising administering at least one CD70-targeting agent to the subject, wherein the subject has at least one AML cell that exhibits a mutation in TET2. The present disclosure provides methods of treating AML in a subject in need thereof, the methods comprising administering at least one CD70-targeting agent to the subject, wherein the subject has a number and/or percentage of AML cells that exhibit a mutation in TET2 that is equal to or greater than a predetermined cutoff value. In some aspects, the preceding methods can further comprise administering to the subject a combination of at least one BCL-2 inhibitor and at least one hypomethylating agent.

TP53-Based Methods

The following methods are based, inter alia, on the discovery (presented herein in the experimental examples) that AML subjects having mutated TP53 may respond more favorably to treatment that comprises the administration of at least one CD70-targeting agent as opposed to a treatment that does not comprise the administration of at least one CD70-targeting agent.

Accordingly, the present disclosure provides methods of treating acute myeloid leukemia (AML) in a subject, the methods comprising: a) determining the mutational status of TP53 in a plurality of cells in a sample from the subject; and b) administering to the subject a first treatment when at least one mutation in TP53 are identified, wherein the first treatment comprises at least one CD70-targeting agent. In some aspects, the method can further comprise administering to the subject a second treatment when no mutations in TP53 are identified, wherein the second treatment does not comprise a CD70-targeting agent.

The present disclosure also provides methods of treating acute myeloid leukemia (AML) in a subject, the methods comprising: a) determining the mutational status of TP53 in a plurality of cells in a sample from the subject; b) determining the number and/or percentage of cells in the plurality of cells that exhibit at least one mutation in TP53 based on step (a); c) comparing the number and/or percentage identified in step (b) to a predetermined cutoff value; and d) administering to the subject a first treatment when the number and/or percentage of cells that exhibit at least one mutation in TP53 is equal to or greater than the predetermined cutoff value, wherein the first treatment comprises at least one CD70-targeting agent. In some aspects, the method further comprises administering to the subject a second treatment when the number and/or percentage of cells that exhibit at least one mutation in TP53 is less than the predetermined cutoff value, wherein the second treatment does not comprise a CD70-targeting agent.

The present disclosure also provides methods of identifying if a subject having AML will be responsive to an AML treatment comprising the administration of at least one CD70-targeting agent, the methods comprising: a) determining the mutational status of TP53 in a plurality of cells in a sample from the subject; and b) identifying that the subject will be responsive to the AML treatment when the presence of at least one mutation in TP53 is identified.

The present disclosure also provides methods of identifying if a subject having AML will be responsive to an AML treatment comprising the administration of at least one CD70-targeting agent, the methods comprising: a) determining the mutational status of TP53 in a plurality of cells in a sample from the subject; b) determining the number and/or percentage of cells in the plurality of cells that exhibit at least one mutation in TP53 based on step (a); c) comparing the number and/or percentage identified in step (b) to a predetermined cutoff value; and d) identifying that the subject will be responsive to the AML treatment when the number and/or percentage of cells that exhibit at least one mutation in TP53 is equal to or greater than the predetermined cutoff value.

The present disclosure provides methods of treating AML in a subject in need thereof, the methods comprising administering at least one CD70-targeting agent to the subject, wherein the subject has at least one AML cell that exhibits a mutation in TP53. The present disclosure provides methods of treating AML in a subject in need thereof, the methods comprising administering at least one CD70-targeting agent to the subject, wherein the subject has a number and/or percentage of AML cells that exhibit a mutation in TP53 that is equal to or greater than a predetermined cutoff value. In some aspects, the preceding methods can further comprise administering to the subject a combination of at least one BCL-2 inhibitor and at least one hypomethylating agent.

### U2AF1-Based Methods

The following methods are based, inter alia, on the discovery (presented herein in the experimental examples) that AML subjects having mutated U2AF1 may respond more favorably to treatment that comprises the administration of at least one CD70-targeting agent as opposed to a treatment that does not comprise the administration of at least one CD70-targeting agent.

Accordingly, the present disclosure provides methods of treating acute myeloid leukemia (AML) in a subject, the methods comprising: a) determining the mutational status of U2AF1 in a plurality of cells in a sample from the subject; and b) administering to the subject a first treatment when at least one mutation in U2AF1 are identified, wherein the first treatment comprises at least one CD70-targeting agent. In some aspects, the method can further comprise administering to the subject a second treatment when no mutations in U2AF1 are identified, wherein the second treatment does not comprise a CD70-targeting agent.

The present disclosure also provides methods of treating acute myeloid leukemia (AML) in a subject, the methods comprising: a) determining the mutational status of U2AF1 in a plurality of cells in a sample from the subject; b) determining the number and/or percentage of cells in the plurality of cells that exhibit at least one mutation in U2AF1 based on step (a); c) comparing the number and/or percentage identified in step (b) to a predetermined cutoff value; and d) administering to the subject a first treatment when the number and/or percentage of cells that exhibit at least one mutation in U2AF1 is equal to or greater than the predetermined cutoff value, wherein the first treatment comprises at least one CD70-targeting agent. In some aspects, the method further comprises administering to the subject a second treatment when the number and/or percentage of cells that exhibit at least one mutation in U2AF1 is less than the predetermined cutoff value, wherein the second treatment does not comprise a CD70-targeting agent.

The present disclosure also provides methods of identifying if a subject having AML will be responsive to an AML treatment comprising the administration of at least one CD70-targeting agent, the methods comprising: a) determining the mutational status of U2AF1 in a plurality of cells in a sample from the subject; and b) identifying that the subject will be responsive to the AML treatment when the presence of at least one mutation in U2AF1 is identified.

The present disclosure also provides methods of identifying if a subject having AML will be responsive to an AML treatment comprising the administration of at least one CD70-targeting agent, the methods comprising: a) determining the mutational status of U2AF1 in a plurality of cells in a sample from the subject; b) determining the number and/or percentage of cells in the plurality of cells that exhibit at least one mutation in U2AF1 based on step (a); c) comparing the number and/or percentage identified in step (b) to a predetermined cutoff value; and d) identifying that the subject will be responsive to the AML treatment when the number and/or percentage of cells that exhibit at least one mutation in U2AF1 is equal to or greater than the predetermined cutoff value.

The present disclosure provides methods of treating AML in a subject in need thereof, the methods comprising administering at least one CD70-targeting agent to the subject, wherein the subject has at least one AML cell that exhibits a mutation in U2AF. The present disclosure provides methods of treating AML in a subject in need thereof, the methods comprising administering at least one CD70-targeting agent to the subject, wherein the subject has a number and/or percentage of AML cells that exhibit a mutation in U2AF1 that is equal to or greater than a predetermined cutoff value.

In some aspects, the preceding methods can further comprise administering to the subject a combination of at least one BCL-2 inhibitor and at least one hypomethylating agent.

### GATA2-Based Methods

The following methods are based, inter alia, on the discovery (presented herein in the experimental examples) that AML subjects having mutated GATA2 may respond favorably to treatments that do not comprise the administration of at least one CD70-targeting agent, and therefore can be excluded from treatment with a CD70-targeting agent.

Accordingly, the present disclosure provides methods of treating acute myeloid leukemia (AML) in a subject, the methods comprising: a) determining the mutational status of GATA2 in a plurality of cells in a sample from the subject; and b) administering to the subject a first treatment when at least one mutation in GATA2 are identified, wherein the first treatment does not comprise at least one CD70-targeting agent. In some aspects, the method can further comprise administering to the subject a second treatment when no mutations in GATA2 are identified, wherein the second treatment comprises at least one CD70-targeting agent.

The present disclosure also provides methods of treating acute myeloid leukemia (AML) in a subject, the methods comprising: a) determining the mutational status of GATA2 in a plurality of cells in a sample from the subject; b) determining the number and/or percentage of cells in the plurality of cells that exhibit at least one mutation in GATA2 based on step (a); c) comparing the number and/or percentage identified in step (b) to a predetermined cutoff value; and d) administering to the subject a first treatment when the number and/or percentage of cells that exhibit at least one mutation in GATA2 is equal to or greater than the predetermined cutoff value, wherein the first treatment does not comprise at least one CD70-targeting agent. In some aspects, the method further comprises administering to the subject a second treatment when the number and/or percentage of cells that exhibit at least one mutation in GATA2 is less than the predetermined cutoff value, wherein the second treatment comprises at least one CD70-targeting agent.

The present disclosure also provides methods of identifying if a subject having AML will be responsive to an AML treatment comprising the administration of at least one CD70-targeting agent, the methods comprising: a) determining the mutational status of GATA2 in a plurality of cells in a sample from the subject; and b) identifying that the subject will be responsive to the AML treatment when the presence no mutations in GATA2 are identified.

The present disclosure also provides methods of identifying if a subject having AML will be responsive to an AML treatment comprising the administration of at least one CD70-targeting agent, the methods comprising: a) determining the mutational status of GATA2 in a plurality of cells in a sample from the subject; b) determining the number and/or percentage of cells in the plurality of cells that exhibit at least one mutation in GATA2 based on step (a); c) comparing the number and/or percentage identified in step (b) to a predetermined cutoff value; and d) identifying that the subject will be responsive to the AML treatment when the number and/or percentage of cells that exhibit at least one mutation in GATA2 is less than the predetermined cutoff value.

The present disclosure provides methods of treating AML in a subject in need thereof, the methods comprising administering at least one CD70-targeting agent to the subject, wherein the subject has no AML cells that exhibit a mutation in GATA2. The present disclosure provides methods of treating AML in a subject in need thereof, the methods comprising administering at least one CD70-targeting agent to the subject, wherein the subject has a number and/or percentage of AML cells that exhibit a mutation in GATA2 that is less than a predetermined cutoff value.

In some aspects, the preceding methods can further comprise administering to the subject a combination of at least one BCL-2 inhibitor and at least one hypomethylating agent. For example, treatments that do not comprise CD-70 targeting agents can comprise at least one BCL-2 inhibitor and at least one hypomethylating agent.

### Inv(3)-Based Methods

The following methods are based, inter alia, on the discovery (presented herein in the experimental examples) that AML subjects having inv(3) phenotypes may respond more favorably to treatment that comprises the administration of at least one CD70-targeting agent as opposed to a treatment that does not comprise the administration of at least one CD70-targeting agent.

Accordingly, the present disclosure provides methods of treating acute myeloid leukemia (AML) in a subject, the methods comprising: a) determining the cytogenetic status of a plurality of cells in a sample from the subject; and b) administering to the subject a first treatment when at least one cell is identified as having an inv(3) phenotype, wherein the first treatment comprises at least one CD70-targeting agent. In some aspects, the method can further comprise administering to the subject a second treatment when no cells having an inv(3) are identified, wherein the second treatment does not comprise a CD70-targeting agent.

The present disclosure also provides methods of treating acute myeloid leukemia (AML) in a subject, the methods comprising: a) determining the cytogenetic status of a plurality of cells in a sample from the subject; b) determining the number and/or percentage of cells in the plurality of cells that exhibit an inv(3) phenotype based on step (a); c) comparing the number and/or percentage identified in step (b) to a predetermined cutoff value; and d) administering to the subject a first treatment when the number and/or percentage of cells that exhibit an inv(3) phenotype is equal to or greater than the predetermined cutoff value, wherein the first treatment comprises at least one CD70-targeting agent. In some aspects, the method can further comprise administering to the subject a second treatment when the number and/or percentage of cells that exhibit an inv(3) phenotype is less than the predetermined cutoff value, wherein the second treatment does not comprise a CD70-targeting agent.

The present disclosure also provides methods of identifying if a subject having AML will be responsive to an AML treatment comprising the administration of at least one CD70-targeting agent, the methods comprising: a) determining the cytogenetic status of a plurality of cells in a sample from the subject; and b) identifying that the subject will be responsive to the AML treatment when the presence of at least cell exhibiting an inv(3) phenotype is identified.

The present disclosure also provides methods of identifying if a subject having AML will be responsive to an AML treatment comprising the administration of at least one CD70-targeting agent, the methods comprising: a) determining the cytogenetic status of a plurality of cells in a sample from the subject; b) determining the number and/or percentage of cells in the plurality of cells that exhibit an inv(3) phenotype based on step (a); c) comparing the number and/or percentage identified in step (b) to a predetermined cutoff value; and d) identifying that the subject will be responsive to the AML treatment when the number and/or percentage of cells that exhibit an inv(3) phenotype is equal to or greater than the predetermined cutoff value.

The present disclosure provides methods of treating AML in a subject in need thereof, the methods comprising administering at least one CD70-targeting agent to the subject, wherein the subject has at least one AML cell that exhibits an inv(3) phenotype. The present disclosure provides methods of treating AML in a subject in need thereof, the methods comprising administering at least one CD70-targeting agent to the subject, wherein the subject has a number and/or percentage of AML cells that exhibit an inv(3) phenotype that is equal to or greater than a predetermined cutoff value.

In some aspects, the preceding methods can further comprise administering to the subject a combination of at least one BCL-2 inhibitor and at least one hypomethylating agent.

### Del7q-Based Methods

The following methods are based, inter alia, on the discovery (presented herein in the experimental examples) that AML subjects having Del7q phenotypes may respond more favorably to treatment that comprises the administration of at least one CD70-targeting agent as opposed to a treatment that does not comprise the administration of at least one CD70-targeting agent.

Accordingly, the present disclosure provides methods of treating acute myeloid leukemia (AML) in a subject, the methods comprising: a) determining the cytogenetic status of a plurality of cells in a sample from the subject; and b) administering to the subject a first treatment when at least one cell is identified as having a Del7q phenotype, wherein the first treatment comprises at least one CD70-targeting agent. In some aspects, the method can further comprise administering to the subject a second treatment when no cells having a Del7q are identified, wherein the second treatment does not comprise a CD70-targeting agent.

The present disclosure also provides methods of treating acute myeloid leukemia (AML) in a subject, the methods comprising: a) determining the cytogenetic status of a plurality of cells in a sample from the subject; b) determining the number and/or percentage of cells in the plurality of cells that exhibit a Del7q phenotype based on step (a); c) comparing the number and/or percentage identified in step (b) to a predetermined cutoff value; and d) administering to the subject a first treatment when the number and/or percentage of cells that exhibit a Del7q phenotype is equal to or greater than the predetermined cutoff value, wherein the first treatment comprises at least one CD70-targeting agent. In some aspects, the method can further comprise administering to the subject a second treatment when the number and/or percentage of cells that exhibit a Del7q phenotype is less than the predetermined cutoff value, wherein the second treatment does not comprise a CD70-targeting agent.

The present disclosure also provides methods of identifying if a subject having AML will be responsive to an AML treatment comprising the administration of at least one CD70-targeting agent, the methods comprising: a) determining the cytogenetic status of a plurality of cells in a sample from the subject; and b) identifying that the subject will be responsive to the AML treatment when the presence of at least cell exhibiting a Del7q phenotype is identified.

The present disclosure also provides methods of identifying if a subject having AML will be responsive to an AML treatment comprising the administration of at least one CD70-targeting agent, the methods comprising: a) determining the cytogenetic status of a plurality of cells in a sample from the subject; b) determining the number and/or percentage of cells in the plurality of cells that exhibit a Del7q phenotype based on step (a); c) comparing the number and/or percentage identified in step (b) to a predetermined cutoff value; and d) identifying that the subject will be responsive to the AML treatment when the number and/or percentage of cells that exhibit a Del7q phenotype is equal to or greater than the predetermined cutoff value.

The present disclosure provides methods of treating AML in a subject in need thereof, the methods comprising administering at least one CD70-targeting agent to the subject, wherein the subject has at least one AML cell that exhibits a Del7q phenotype. The present disclosure provides methods of treating AML in a subject in need thereof, the methods comprising administering at least one CD70-targeting agent to the subject, wherein the subject has a number and/or percentage of AML cells that exhibit a Del7q phenotype that is equal to or greater than a predetermined cutoff value.

In some aspects, the preceding methods can further comprise administering to the subject a combination of at least one BCL-2 inhibitor and at least one hypomethylating agent.

### 17⁻-Based Methods

The following methods are based, inter alia, on the discovery (presented herein in the experimental examples) that AML subjects having 17⁻ phenotypes may respond more favorably to treatment that comprises the administration of at least one CD70-targeting agent as opposed to a treatment that does not comprise the administration of at least one CD70-targeting agent.

Accordingly, the present disclosure provides methods of treating acute myeloid leukemia (AML) in a subject, the methods comprising: a) determining the cytogenetic status of a plurality of cells in a sample from the subject; and b) administering to the subject a first treatment when at least one cell is identified as having a 17⁻ phenotype, wherein the first treatment comprises at least one CD70-targeting agent. In some aspects, the method can further comprise administering to the subject a second treatment when no cells having a 17⁻ are identified, wherein the second treatment does not comprise a CD70-targeting agent.

The present disclosure also provides methods of treating acute myeloid leukemia (AML) in a subject, the methods comprising: a) determining the cytogenetic status of a plurality of cells in a sample from the subject; b) determining the number and/or percentage of cells in the plurality of cells that exhibit a 17⁻ phenotype based on step (a); c) comparing the number and/or percentage identified in step (b) to a predetermined cutoff value; and d) administering to the subject a first treatment when the number and/or percentage of cells that exhibit a 17⁻ phenotype is equal to or greater than the predetermined cutoff value, wherein the first treatment comprises at least one CD70-targeting agent. In some aspects, the method can further comprise administering to the subject a second treatment when the number and/or percentage of cells that exhibit a 17⁻ phenotype is less than the predetermined cutoff value, wherein the second treatment does not comprise a CD70-targeting agent.

The present disclosure also provides methods of identifying if a subject having AML will be responsive to an AML treatment comprising the administration of at least one CD70-targeting agent, the methods comprising: a) determining the cytogenetic status of a plurality of cells in a sample from the subject; and b) identifying that the subject will be responsive to the AML treatment when the presence of at least cell exhibiting a 17⁻ phenotype is identified.

The present disclosure also provides methods of identifying if a subject having AML will be responsive to an AML treatment comprising the administration of at least one CD70-targeting agent, the methods comprising: a) determining the cytogenetic status of a plurality of cells in a sample from the subject; b) determining the number and/or percentage of cells in the plurality of cells that exhibit a 17⁻ phenotype based on step (a); c) comparing the number and/or percentage identified in step (b) to a predetermined cutoff value; and d) identifying that the subject will be responsive to the AML treatment when the number and/or percentage of cells that exhibit a 17⁻ phenotype is equal to or greater than the predetermined cutoff value.

The present disclosure provides methods of treating AML in a subject in need thereof, the methods comprising administering at least one CD70-targeting agent to the subject, wherein the subject has at least one AML cell that exhibits a 17⁻ phenotype. The present disclosure provides methods of treating AML in a subject in need thereof, the methods comprising administering at least one CD70-targeting agent to the subject, wherein the subject has a number and/or percentage of AML cells that exhibit a T phenotype that is equal to or greater than a predetermined cutoff value. In some aspects, the preceding methods can further comprise administering to the subject a combination of at least one BCL-2 inhibitor and at least one hypomethylating agent.

### 7 Centromere-Based Methods

The following methods are based, inter alia, on the discovery (presented herein in the experimental examples) that AML subjects having 7 centromere phenotypes may respond more favorably to treatment that comprises the administration of at least one CD70-targeting agent as opposed to a treatment that does not comprise the administration of at least one CD70-targeting agent.

In some aspects, a 7 centromere phenotype is a 7 centromere gain phenotype. In some aspects, a 7 centromere phenotype is a 7 centromere loss phenotype.

Accordingly, the present disclosure provides methods of treating acute myeloid leukemia (AML) in a subject, the methods comprising: a) determining the cytogenetic status of a plurality of cells in a sample from the subject; and b) administering to the subject a first treatment when at least one cell is identified as having a 7 centromere phenotype, wherein the first treatment comprises at least one CD70-targeting agent. In some aspects, the method can further comprise administering to the subject a second treatment when no cells having a 7 centromere are identified, wherein the second treatment does not comprise a CD70-targeting agent.

The present disclosure also provides methods of treating acute myeloid leukemia (AML) in a subject, the methods comprising: a) determining the cytogenetic status of a plurality of cells in a sample from the subject; b) determining the number and/or percentage of cells in the plurality of cells that exhibit a 7 centromere phenotype based on step (a); c) comparing the number and/or percentage identified in step (b) to a predetermined cutoff value; and d) administering to the subject a first treatment when the number and/or percentage of cells that exhibit a 7 centromere phenotype is equal to or greater than the predetermined cutoff value, wherein the first treatment comprises at least one CD70-targeting agent. In some aspects, the method can further comprise administering to the subject a second treatment when the number and/or percentage of cells that exhibit a 7 centromere phenotype is less than the predetermined cutoff value, wherein the second treatment does not comprise a CD70-targeting agent.

The present disclosure also provides methods of identifying if a subject having AML will be responsive to an AML treatment comprising the administration of at least one CD70-targeting agent, the methods comprising: a) determining the cytogenetic status of a plurality of cells in a sample from the subject; and b) identifying that the subject will be responsive to the AML treatment when the presence of at least cell exhibiting a 7 centromere phenotype is identified.

The present disclosure also provides methods of identifying if a subject having AML will be responsive to an AML treatment comprising the administration of at least one CD70-targeting agent, the methods comprising: a) determining the cytogenetic status of a plurality of cells in a sample from the subject; b) determining the number and/or percentage of cells in the plurality of cells that exhibit a 7 centromere phenotype based on step (a); c) comparing the number and/or percentage identified in step (b) to a predetermined cutoff value; and d) identifying that the subject will be responsive to the AML treatment when the number and/or percentage of cells that exhibit a 7 centromere phenotype is equal to or greater than the predetermined cutoff value.

The present disclosure provides methods of treating AML in a subject in need thereof, the methods comprising administering at least one CD70-targeting agent to the subject, wherein the subject has at least one AML cell that exhibits a 7 centromere phenotype. The present disclosure provides methods of treating AML in a subject in need thereof, the methods comprising administering at least one CD70-targeting agent to the subject, wherein the subject has a number and/or percentage of AML cells that exhibit a 7 centromere phenotype that is equal to or greater than a predetermined cutoff value. In some aspects, the preceding methods can further comprise administering to the subject a combination of at least one BCL-2 inhibitor and at least one hypomethylating agent.

### EGR1⁻-Based Methods

The following methods are based, inter alia, on the discovery (presented herein in the experimental examples) that AML subjects having EGR1⁻ phenotypes may respond more favorably to treatment that comprises the administration of at least one CD70-targeting agent as opposed to a treatment that does not comprise the administration of at least one CD70-targeting agent.

Accordingly, the present disclosure provides methods of treating acute myeloid leukemia (AML) in a subject, the methods comprising: a) determining the cytogenetic status of a plurality of cells in a sample from the subject; and b) administering to the subject a first treatment when at least one cell is identified as having an EGR1⁻ phenotype, wherein the first treatment comprises at least one CD70-targeting agent. In some aspects, the method can further comprise administering to the subject a second treatment when no cells having an EGR1⁻ are identified, wherein the second treatment does not comprise a CD70-targeting agent.

The present disclosure also provides methods of treating acute myeloid leukemia (AML) in a subject, the methods comprising: a) determining the cytogenetic status of a plurality of cells in a sample from the subject; b) determining the number and/or percentage of cells in the plurality of cells that exhibit an EGR1⁻ phenotype based on step (a); c) comparing the number and/or percentage identified in step (b) to a predetermined cutoff value; and d) administering to the subject a first treatment when the number and/or percentage of cells that exhibit an EGR1⁻ phenotype is equal to or greater than the predetermined cutoff value, wherein the first treatment comprises at least one CD70-targeting agent. In some aspects, the method can further comprise administering to the subject a second treatment when the number and/or percentage of cells that exhibit an EGR1⁻ phenotype is less than the predetermined cutoff value, wherein the second treatment does not comprise a CD70-targeting agent.

The present disclosure also provides methods of identifying if a subject having AML will be responsive to an AML treatment comprising the administration of at least one CD70-targeting agent, the methods comprising: a) determining the cytogenetic status of a plurality of cells in a sample from the subject; and b) identifying that the subject will be responsive to the AML treatment when the presence of at least cell exhibiting an EGR1⁻ phenotype is identified.

The present disclosure also provides methods of identifying if a subject having AML will be responsive to an AML treatment comprising the administration of at least one CD70-targeting agent, the methods comprising: a) determining the cytogenetic status of a plurality of cells in a sample from the subject; b) determining the number and/or percentage of cells in the plurality of cells that exhibit an EGR1⁻ phenotype based on step (a); c) comparing the number and/or percentage identified in step (b) to a predetermined cutoff value; and d) identifying that the subject will be responsive to the AML treatment when the number and/or percentage of cells that exhibit an EGR1⁻ phenotype is equal to or greater than the predetermined cutoff value.

The present disclosure provides methods of treating AML in a subject in need thereof, the methods comprising administering at least one CD70-targeting agent to the subject, wherein the subject has at least one AML cell that exhibits an EGR1⁻ phenotype. The present disclosure provides methods of treating AML in a subject in need thereof, the methods comprising administering at least one CD70-targeting agent to the subject, wherein the subject has a number and/or percentage of AML cells that exhibit an EGR1⁻ phenotype that is equal to or greater than a predetermined cutoff value. In some aspects, the preceding methods can further comprise administering to the subject a combination of at least one BCL-2 inhibitor and at least one hypomethylating agent.

### CD2-Based Methods

The following methods are based, inter alia, on the discovery (presented herein in the experimental examples) that AML subjects having CD2+ phenotypes respond poorly to treatment with CD70-targeting agents, while subjects who are CD2− respond well to treatment with CD70-targeting agents.

Accordingly, the present disclosure provides methods of treating acute myeloid leukemia (AML) in a subject, the methods comprising: a) measuring the expression of at least CD2 in a plurality of cells in a sample from the subject; b) identifying the presence of at least one CD2+ cell based on the expression measured in step (a); and c) administering to the subject a first treatment when no CD2+ cells are identified, wherein the first treatment comprises at least one CD70-targeting agent; or administering to the subject a second treatment when CD2+ cells are identified, wherein the second treatment does not comprise a CD70-targeting agent.

The present disclosure also provides methods of treating acute myeloid leukemia (AML) in a subject, the methods comprising: a) measuring the expression of at least CD2 in a plurality of cells in a sample from the subject; b) identifying the number and/or percentage of CD2+ cells in the plurality of cells based on the expression measured in step (a); c) comparing the number and/or percentage of CD2+ cells identified in step (b) to a predetermined cutoff value; and d) administering to the subject a first treatment when the number and/or percentage of cells is less than the predetermined cutoff value, wherein the first treatment comprises at least one CD70-targeting agent; or administering to the subject a second treatment when number and/or percentage of the CD2+ cells is equal to or greater than the predetermined cutoff value, wherein the second treatment does not comprise a CD70-targeting agent.

The present disclosure also provides methods of identifying if a subject having AML will be responsive to an AML treatment comprising the administration of at least one CD70-targeting agent, the methods comprising: a) measuring the expression of at least CD2 in a plurality of cells in a sample from the subject; b) identifying the presence of at least one CD2+ cell based on the expression measured in step (a); and c) identifying that the subject will not be responsive to the AML treatment when the presence of at least one CD2+ cell is identified; or identifying that the subject will be responsive to the AML treatment when no CD2+ cells are identified.

The present disclosure also provides methods of identifying if a subject having AML will be responsive to an AML treatment comprising the administration of at least one CD70-targeting agent, the methods comprising: a) measuring the expression of at least CD2 in a plurality of cells in a sample from the subject; b) identifying the number and/or percentage of CD2+ cells in the plurality of cells based on the expression measured in step (a); c) comparing the number and/or percentage of CD2+ cells identified in step (b) to a predetermined cutoff value; and d) identifying that the subject will not be responsive to the AML treatment when the number and/or percentage of CD2+ cells is equal to or greater than the predetermined cutoff value; or identifying that the subject will be responsive to the AML treatment when the number and/or percentage of CD2+ cells is less than predetermined cutoff value.

In some aspects of the preceding methods, the first treatment can further comprise a combination of at least one BCL-2 inhibitor and at least one hypomethylating agent.

In some aspects of the preceding methods, the second treatment can further comprise a combination of at least one BCL-2 inhibitor and at least one hypomethylating agent.

In some aspects of the preceding methods, the AML treatment can further comprise the administration of a combination of at least one BCL-2 inhibitor and at least one hypomethylating agent.

The preceding methods can further comprise a step of providing a treatment recommendation to a clinician and/or subject. Accordingly, if a subject is identified as a subject who will not be responsive to a treatment comprising a CD70-targeting agent, the method can further comprise providing a treatment recommendation that comprises administering a treatment that does not include a CD70-targeting agent. If a subject is identified as a subject that will be responsive to treatment with a CD70-targeting agent, then the method can further comprise providing a treatment recommendation comprising administering at least one CD70-targeting agent to the subject.

The present disclosure provides methods of treating AML in a subject in need thereof, the methods comprising administering at least one CD70-targeting agent to the subject, wherein the subject has no AML cells that are CD2+. The present disclosure provides methods of treating AML in a subject in need thereof, the methods comprising administering at least one CD70-targeting agent to the subject, wherein the subject has a number and/or percentage of CD2+ AML cells that is less than a predetermined cutoff value. In some aspects, the preceding methods can further comprise administering to the subject a combination of at least one BCL-2 inhibitor and at least one hypomethylating agent.

### NF1-Based Methods

The following methods are based, inter alia, on the discovery (presented herein in the experimental examples) that AML subjects having mutated NF1 respond poorly to treatment with CD70-targeting agents, while subjects who have wildtype NF1 respond well to treatment with CD70-targeting agents.

Accordingly, the present disclosure provides methods of treating acute myeloid leukemia (AML) in a subject, the methods comprising: a) determining the presence the mutational status of NF1 in a plurality of cells in a sample from the subject; and b) administering to the subject a first treatment when no mutations in NF1 are identified, wherein the first treatment comprises at least one CD70-targeting agent; or administering to the subject a second treatment when at least one mutation in NF1 is identified, wherein the second treatment does not comprise a CD70-targeting agent.

The present disclosure also provides methods of treating acute myeloid leukemia (AML) in a subject, the methods comprising: a) determining the mutational status of NF1 in a plurality of cells in a sample from the subject; b) determining the number and/or percentage of cells in the plurality of cells that exhibit at least one mutation in NF1 based on step (a); c) comparing the number and/or percentage identified in step (b) to a predetermined cutoff value; and d) administering to the subject a first treatment when the number and/or percentage of cells that exhibit at least one mutation in NF1 is less than the predetermined cutoff value, wherein the first treatment comprises at least one CD70-targeting agent; or administering to the subject a second treatment when the number and/or percentage of cells that exhibit at least one mutation in NF1 is equal to or greater than the predetermined cutoff value, wherein the second treatment does not comprise a CD70-targeting agent.

The present disclosure also provides methods of identifying if a subject having AML will be responsive to an AML treatment comprising the administration of at least one CD70-targeting agent, the methods comprising: a) determining the mutational status of NF1 in a plurality of cells in a sample from the subject; and b) identifying that the subject will not be responsive to the AML treatment when the presence of at least one mutation in NF1 is identified; or identifying that the subject will be responsive to the AML treatment when no mutations in NF1 are identified.

The present disclosure also provides methods of identifying if a subject having AML will be responsive to an AML treatment comprising the administration of at least one CD70-targeting agent, the methods comprising: a) determining the mutational status of NF1 in a plurality of cells in a sample from the subject; b) determining the number and/or percentage of cells in the plurality of cells that exhibit at least one mutation in NF1 based on step (a); c) comparing the number and/or percentage identified in step (b) to a predetermined cutoff value; and d) identifying that the subject will not be responsive to the AML treatment when the number and/or percentage of cells that exhibit at least one mutation in NF1 is equal to or greater than the predetermined cutoff value; or identifying that the subject will be responsive to the AML treatment when the number and/or percentage of cells that exhibit at least one mutation in NF1 is less than the predetermined cutoff value.

In some aspects of the preceding methods, the first treatment can further comprise a combination of at least one BCL-2 inhibitor and at least one hypomethylating agent.

In some aspects of the preceding methods, the second treatment can further comprise a combination of at least one BCL-2 inhibitor and at least one hypomethylating agent.

In some aspects of the preceding methods, the AML treatment can further comprise the administration of a combination of at least one BCL-2 inhibitor and at least one hypomethylating agent.

The preceding methods can further comprise a step of providing a treatment recommendation to a clinician and/or subject. Accordingly, if a subject is identified as a subject who will not be responsive to a treatment comprising a CD70-targeting agent, the method can further comprise providing a treatment recommendation that comprises administering a treatment that does not include a CD70-targeting agent. If a subject is identified as a subject that will be responsive to treatment with a CD70-targeting agent, then the method can further comprise providing a treatment recommendation comprising administering at least one CD70-targeting agent to the subject.

The present disclosure provides methods of treating AML in a subject in need thereof, the methods comprising administering at least one CD70-targeting agent to the subject, wherein the subject has no AML cells that exhibit a mutation in NF1. The present disclosure provides methods of treating AML in a subject in need thereof, the methods comprising administering at least one CD70-targeting agent to the subject, wherein the subject has a number and/or percentage of AML cells that exhibit a mutation in NF1 that is less than a predetermined cutoff value. In some aspects, the preceding methods can further comprise administering to the subject a combination of at least one BCL-2 inhibitor and at least one hypomethylating agent.

### TP53-Based Methods

The following methods are based, inter alia, on the discovery (presented herein in the experimental examples) that AML subjects having mutated TP53 respond poorly to treatment with CD70-targeting agents, while subjects who have wildtype TP53 respond well to treatment with CD70-targeting agents.

Accordingly, the present disclosure provides methods of treating acute myeloid leukemia (AML) in a subject, the methods comprising: a) determining the presence the mutational status of TP53 in a plurality of cells in a sample from the subject; and b) administering to the subject a first treatment when no mutations in TP53 are identified, wherein the first treatment comprises at least one CD70-targeting agent; or administering to the subject a second treatment when at least one mutation in TP53 is identified, wherein the second treatment does not comprise a CD70-targeting agent.

The present disclosure also provides methods of treating acute myeloid leukemia (AML) in a subject, the methods comprising: a) determining the mutational status of TP53 in a plurality of cells in a sample from the subject; b) determining the number and/or percentage of cells in the plurality of cells that exhibit at least one mutation in TP53 based on step (a); c) comparing the number and/or percentage identified in step (b) to a predetermined cutoff value; and d) administering to the subject a first treatment when the number and/or percentage of cells that exhibit at least one mutation in TP53 is less than the predetermined cutoff value, wherein the first treatment comprises at least one CD70-targeting agent; or administering to the subject a second treatment when the number and/or percentage of cells that exhibit at least one mutation in TP53 is equal to or greater than the predetermined cutoff value, wherein the second treatment does not comprise a CD70-targeting agent.

The present disclosure also provides methods of identifying if a subject having AML will be responsive to an AML treatment comprising the administration of at least one CD70-targeting agent, the methods comprising: a) determining the mutational status of TP53 in a plurality of cells in a sample from the subject; and b) identifying that the subject will not be responsive to the AML treatment when the presence of at least one mutation in TP53 is identified; or identifying that the subject will be responsive to the AML treatment when no mutations in TP53 are identified.

The present disclosure also provides methods of identifying if a subject having AML will be responsive to an AML treatment comprising the administration of at least one CD70-targeting agent, the methods comprising: a) determining the mutational status of TP53 in a plurality of cells in a sample from the subject; b) determining the number and/or percentage of cells in the plurality of cells that exhibit at least one mutation in TP53 based on step (a); c) comparing the number and/or percentage identified in step (b) to a predetermined cutoff value; and d) identifying that the subject will not be responsive to the AML treatment when the number and/or percentage of cells that exhibit at least one mutation in TP53 is equal to or greater than the predetermined cutoff value; or identifying that the subject will be responsive to the AML treatment when the number and/or percentage of cells that exhibit at least one mutation in TP53 is less than the predetermined cutoff value.

In some aspects of the preceding methods, the first treatment can further comprise a combination of at least one BCL-2 inhibitor and at least one hypomethylating agent.

In some aspects of the preceding methods, the second treatment can further comprise a combination of at least one BCL-2 inhibitor and at least one hypomethylating agent.

In some aspects of the preceding methods, the AML treatment can further comprise the administration of a combination of at least one BCL-2 inhibitor and at least one hypomethylating agent.

The preceding methods can further comprise a step of providing a treatment recommendation to a clinician and/or subject. Accordingly, if a subject is identified as a subject who will not be responsive to a treatment comprising a CD70-targeting agent, the method can further comprise providing a treatment recommendation that comprises administering a treatment that does not include a CD70-targeting agent. If a subject is identified as a subject that will be responsive to treatment with a CD70-targeting agent, then the method can further comprise providing a treatment recommendation comprising administering at least one CD70-targeting agent to the subject.

The present disclosure provides methods of treating AML in a subject in need thereof, the methods comprising administering at least one CD70-targeting agent to the subject, wherein the subject has no AML cells that exhibit a mutation in TP53. The present disclosure provides methods of treating AML in a subject in need thereof, the methods comprising administering at least one CD70-targeting agent to the subject, wherein the subject has a number and/or percentage of AML cells that exhibit a mutation in TP53 that is less than a predetermined cutoff value. In some aspects, the preceding methods can further comprise administering to the subject a combination of at least one BCL-2 inhibitor and at least one hypomethylating agent.

### Chromosome 7-Based Methods

The following methods are based, inter alia, on the discovery (presented herein in the experimental examples) that AML subjects having deleted chromosome 7 status respond poorly to treatment with CD70-targeting agents, while subjects who have normal chromosome 7 status respond well to treatment with CD70-targeting agents.

Accordingly, the present disclosure provides methods of treating acute myeloid leukemia (AML) in a subject, the methods comprising: a) determining the chromosome 7 status in a plurality of cells in a sample from the subject; and b) administering to the subject a first treatment when no chromosome 7 abnormalities are identified, wherein the first treatment comprises at least one CD70-targeting agent; or administering to the subject a second treatment when at least one chromosome 7 abnormality is identified, wherein the second treatment does not comprise a CD70-targeting agent.

The present disclosure also provides methods of treating acute myeloid leukemia (AML) in a subject, the methods comprising: a) determining the chromosome 7 status in a plurality of cells in a sample from the subject; b) determining the number and/or percentage of cells in the plurality of cells that exhibit at least chromosome 7 abnormality based on step (a); c) comparing the number and/or percentage identified in step (b) to a predetermined cutoff value; and d) administering to the subject a first treatment when the number and/or percentage of cells that exhibit at least one chromosome 7 abnormality is less than the predetermined cutoff value, wherein the first treatment comprises at least one CD70-targeting agent; or administering to the subject a second treatment when the number and/or percentage of cells that exhibit at least one chromosome 7 abnormality is equal to or greater than the predetermined cutoff value, wherein the second treatment does not comprise a CD70-targeting agent.

The present disclosure also provides methods of identifying if a subject having AML will be responsive to an AML treatment comprising the administration of at least one CD70-targeting agent, the method comprising: a) determining the chromosome 7 status in a plurality of cells in a sample from the subject; and b) identifying that the subject will not be responsive to the AML treatment when the presence of at least one chromosome 7 abnormality is identified; or identifying that the subject will be responsive to the AML treatment when no chromosome 7 abnormalities are identified.

The present disclosure also provides methods of identifying if a subject having AML will be responsive to an AML treatment comprising the administration of at least one CD70-targeting agent, the method comprising: a) determining the chromosome 7 status in a plurality of cells in a sample from the subject; b) determining the number and/or percentage of cells in the plurality of cells that exhibit at least chromosome 7 abnormality based on step (a); c) comparing the number and/or percentage identified in step (b) to a predetermined cutoff value; and d) identifying that the subject will not be responsive to the AML treatment when the number and/or percentage of cells that exhibit at least one chromosome abnormality is equal to or greater than the predetermined cutoff value; or identifying that the subject will be responsive to the AML treatment when no the number and/or percentage of cells that exhibit chromosome 7 abnormalities is less than the predetermined cutoff value.

In some aspects of the preceding methods, the wherein the at least one chromosome 7 abnormality can be a chromosome 7 deletion.

In some aspects of the preceding methods, the first treatment can further comprise a combination of at least one BCL-2 inhibitor and at least one hypomethylating agent.

In some aspects of the preceding methods, the second treatment can further comprise a combination of at least one BCL-2 inhibitor and at least one hypomethylating agent.

In some aspects of the preceding methods, the AML treatment can further comprise the administration of a combination of at least one BCL-2 inhibitor and at least one hypomethylating agent.

The preceding methods can further comprise a step of providing a treatment recommendation to a clinician and/or subject. Accordingly, if a subject is identified as a subject who will not be responsive to a treatment comprising a CD70-targeting agent, the method can further comprise providing a treatment recommendation that comprises administering a treatment that does not include a CD70-targeting agent. If a subject is identified as a subject that will be responsive to treatment with a CD70-targeting agent, then the method can further comprise providing a treatment recommendation comprising administering at least one CD70-targeting agent to the subject.

The present disclosure provides methods of treating AML in a subject in need thereof, the methods comprising administering at least one CD70-targeting agent to the subject, wherein the subject has no AML cells that exhibit a chromosome 7 abnormality. The present disclosure provides methods of treating AML in a subject in need thereof, the methods comprising administering at least one CD70-targeting agent to the subject, wherein the subject has a number and/or percentage of AML cells that exhibit a chromosome 7 abnormality that is less than a predetermined cutoff value. In some aspects, the preceding methods can further comprise administering to the subject a combination of at least one BCL-2 inhibitor and at least one hypomethylating agent.

### CD7-Based Methods

The following methods are based, inter alia, on the discovery (presented herein in the experimental examples) that AML subjects having CD7+ phenotypes respond poorly to treatment with a combination of at least one BCL-2 inhibitor and at least one hypomethylating agent, while subjects who are CD7− respond well to treatment with the combination, and that the CD7+ subjects can benefit from a treatment regimen that includes the administration of at least one CD70-targeting agent.

Accordingly, the present disclosure provides methods of treating acute myeloid leukemia (AML) in a subject, the methods comprising: a) measuring the expression of at least CD7 in a plurality of cells in a sample from the subject; b) identifying the presence of at least one CD7+ cell based on the expression measured in step (a); and c) administering to the subject a first treatment when no CD7+ cells are identified, wherein the first treatment comprises a combination of at least one BCL-2 inhibitor and at least one hypomethylating agent; or administering to the subject a second treatment when CD7+ cells are identified, wherein the second treatment comprises a combination of at least one BCL-2 inhibitor, at least one hypomethylating agent and at least one additional agent, preferably wherein the additional agent comprises at least one CD70-targeting agent.

The present disclosure also provides methods of treating acute myeloid leukemia (AML) in a subject, the methods comprising: a) measuring the expression of at least CD7 in a plurality of cells in a sample from the subject; b) determining the number and/or percentage of CD7+ cells in the plurality of cells based on the expression measured in step (a); c) comparing the number and/or percentage to a predetermined cutoff value; and d) administering to the subject a first treatment when the number and/or percentage of CD7+ cells is less than the predetermined cutoff value, wherein the first treatment comprises a combination of at least one BCL-2 inhibitor and at least one hypomethylating agent; or administering to the subject a second treatment when the number and/or percentage of CD7+ cells is equal to or greater than the predetermined cutoff value, wherein the second treatment comprises a combination of at least one BCL-2 inhibitor, at least one hypomethylating agent and at least one additional agent, preferably wherein the additional agent comprises at least one CD70-targeting agent.

The present disclosure also provides methods of identifying if a subject having AML will be responsive to an AML treatment comprising the administration of a combination of at least one BCL-2 inhibitor and at least one hypomethylating agent, the methods comprising: a) measuring the expression of at least CD7 in a plurality of cells in a sample from the subject; b) identifying the presence of at least one CD7+ cell based on the expression measured in step (a); and c) identifying that the subject will not be responsive to the AML treatment when the presence of at least one CD7+ cell is identified; or identifying that the subject will be responsive to the AML treatment when no CD7+ cells are identified.

The present disclosure also provides methods of identifying if a subject having AML will be responsive to an AML treatment comprising the administration of a combination of at least one BCL-2 inhibitor and at least one hypomethylating agent, the methods comprising: a) measuring the expression of at least CD7 in a plurality of cells in a sample from the subject; b) determining the number and/or percentage of CD7+ cells in the plurality of cells based on the expression measured in step (a); c) comparing the number and/or percentage to a predetermined cutoff value; and d) identifying that the subject will not be responsive to the AML treatment when the number and/or percentage of CD7+ cells is equal to or greater than the predetermined cutoff value; or identifying that the subject will be responsive to the AML treatment when the number and/or percentage of CD7+ cells is less than the predetermined cutoff value.

In some aspects, the preceding methods can further comprise providing a treatment recommendation to a subject identified as a subject that will not be responsive to treatment with a combination of at least one BCL-2 inhibitor and at least one hypomethylating agent, wherein the treatment recommendation comprises recommending administering at least one CD70-targeting agent, optionally wherein the treatment recommendation further comprises recommending administering the at least one CD70-targeting agent in combination with at least one BCL-2 inhibitor and at least one hypomethylating agent, and/or

In some aspects, the preceding methods can further comprise administering to a subject identified as a subject that will not be responsive to treatment with a combination of at least one BCL-2 inhibitor and at least one hypomethylating agent a treatment comprising at least one CD70-targeting agent, optionally in combination with at least one BCL-2 inhibitor and at least one hypomethylating agent.

The present disclosure provides methods of treating AML in a subject in need thereof, the methods comprising administering at least one CD70-targeting agent to the subject, wherein the subject has AML cells that are CD7+. The present disclosure provides methods of treating AML in a subject in need thereof, the methods comprising administering at least one CD70-targeting agent to the subject, wherein the subject has a number and/or percentage of CD7+ AML cells that equal to or greater than a predetermined cutoff value. In some aspects, the preceding methods can further comprise administering to the subject a combination of at least one BCL-2 inhibitor and at least one hypomethylating agent.

### KRAS-Based Methods

The following methods are based, inter alia, on the discovery (presented herein in the experimental examples) that AML subjects having mutated KRAS respond poorly to treatment with a combination of at least one BCL-2 inhibitor and at least one hypomethylating agent, while subjects who have wildtype KRAS respond well to treatment with a combination of at least one BCL-2 inhibitor and at least one hypomethylating agent, and that subject having KRAS mutations can benefit from a treatment regimen that includes the administration of at least one CD70-targeting agent.

Accordingly, the present disclosure provides methods of treating acute myeloid leukemia (AML) in a subject, the methods comprising: a) determining the mutational status of KRAS in a plurality of cells in a sample from the subject, and b) administering to the subject a first treatment when no mutations in KRAS are identified, wherein the first treatment comprises a combination of at least one BCL-2 inhibitor and at least one hypomethylating agent; or administering to the subject a second treatment when at least one mutation in KRAS is identified, wherein the second treatment comprises a combination of at least one BCL-2 inhibitor, at least one hypomethylating agent and at least one additional agent, preferably wherein the additional agent comprises at least one CD70-targeting agent.

The present disclosure also provides methods of treating acute myeloid leukemia (AML) in a subject, the methods comprising: a) determining the mutational status of KRAS in a plurality of cells in a sample from the subject; b) determining the number and/or percentage of cells in the plurality of cells that exhibit at least one KRAS mutation based on step (a); c) comparing the number and/or percentage from step (b) to a predetermined cutoff value; and d) administering to the subject a first treatment when the number and/or percentage of cells that exhibit at least one KRAS mutation is less than the predetermined cutoff value, wherein the first treatment comprises a combination of at least one BCL-2 inhibitor and at least one hypomethylating agent; or administering to the subject a second treatment when the number and/or percentage of cells that exhibit at least one KRAS mutation is equal to or greater than the predetermined cutoff value, wherein the second treatment comprises a combination of at least one BCL-2 inhibitor, at least one hypomethylating agent and at least one additional agent, preferably wherein the additional agent comprises at least one CD70-targeting agent.

The present disclosure also provides methods of identifying if a subject having AML will be responsive to an AML treatment comprising the administration of a combination of at least one BCL-2 inhibitor and at least one hypomethylating agent, the methods comprising: a) determining the mutational status of KRAS in a plurality of cells in a sample from the subject; and b) identifying that the subject will not be responsive to the AML treatment when the presence of at least one mutation in KRAS is identified; or identifying that the subject will be responsive to the AML treatment when no mutations in KRAS are identified.

The present disclosure also provides methods of identifying if a subject having AML will be responsive to an AML treatment comprising the administration of a combination of at least one BCL-2 inhibitor and at least one hypomethylating agent, the methods comprising: a) determining the mutational status of KRAS in a plurality of cells in a sample from the subject; b) determining the number and/or percentage of cells in the plurality of cells that exhibit at least one KRAS mutation based on step (a); c) comparing the number and/or percentage from step (b) to a predetermined cutoff value; and d) identifying that the subject will not be responsive to the AML treatment when the number and/or percentage of cells that exhibit at least one mutation in KRAS is equal to or greater than the predetermined cutoff value; or identifying that the subject will be responsive to the AML treatment when the number and/or percentage of cells that exhibit at least one mutation in KRAS is less than the predetermined cutoff value.

In some aspects, the preceding methods can further comprise providing a treatment recommendation to a subject identified as a subject that will not be responsive to treatment with a combination of at least one BCL-2 inhibitor and at least one hypomethylating agent, wherein the treatment recommendation comprises recommending administering at least one CD70-targeting agent, optionally wherein the treatment recommendation further comprises recommending administering the at least one CD70-targeting agent in combination with at least one BCL-2 inhibitor and at least one hypomethylating agent; and/or

In some aspects, the preceding methods can further comprise administering to a subject identified as a subject that will not be responsive to treatment with a combination of at least one BCL-2 inhibitor and at least one hypomethylating agent a treatment comprising at least one CD70-targeting agent, optionally in combination with at least one BCL-2 inhibitor and at least one hypomethylating agent.

The present disclosure provides methods of treating AML in a subject in need thereof, the methods comprising administering at least one CD70-targeting agent to the subject, wherein the subject has at least one AML cell that exhibits a mutation in KRAS. The present disclosure provides methods of treating AML in a subject in need thereof, the methods comprising administering at least one CD70-targeting agent to the subject, wherein the subject has a number and/or percentage of AML cells that exhibit a mutation in KRAS that is equal to or greater than a predetermined cutoff value. In some aspects, the preceding methods can further comprise administering to the subject a combination of at least one BCL-2 inhibitor and at least one hypomethylating agent.

### PTPN11-Based Methods

The following methods are based, inter alia, on the discovery (presented herein in the experimental examples) that AML subjects having mutated PTPN11 respond poorly to treatment with a combination of at least one BCL-2 inhibitor and at least one hypomethylating agent, while subjects who have wildtype PTPN11 respond well to treatment with a combination of at least one BCL-2 inhibitor and at least one hypomethylating agent, and that subject having PTPN11 mutations can benefit from a treatment regimen that includes the administration of at least one CD70-targeting agent.

Accordingly, the present disclosure provides methods of treating acute myeloid leukemia (AML) in a subject, the methods comprising: a) determining the mutational status of PTPN11 in a plurality of cells in a sample from the subject; and b) administering to the subject a first treatment when no mutations in PTPN11 are identified, wherein the first treatment comprises a combination of at least one BCL-2 inhibitor and at least one hypomethylating agent; or administering to the subject a second treatment when at least one mutation in PTPN11 is identified, wherein the second treatment comprises a combination of at least one BCL-2 inhibitor, at least one hypomethylating agent and at least one additional agent, preferably wherein the additional agent comprises at least one CD70-targeting agent.

The present disclosure also provides methods of treating acute myeloid leukemia (AML) in a subject, the methods comprising: a) determining the mutational status of PTPN11 in a plurality of cells in a sample from the subject; b) determining the number and/or percentage of cells in the plurality of cells that exhibit at least one PTPN11 mutation based on step (a); c) comparing the number and/or percentage from step (b) to a predetermined cutoff value; and d) administering to the subject a first treatment when the number and/or percentage of cells that exhibit at least one PTPN11 mutation is less than the predetermined cutoff value, wherein the first treatment comprises a combination of at least one BCL-2 inhibitor and at least one hypomethylating agent; or administering to the subject a second treatment when the number and/or percentage of cells that exhibit at least one PTPN11 mutation is equal to or greater than the predetermined cutoff value, wherein the second treatment comprises a combination of at least one BCL-2 inhibitor, at least one hypomethylating agent and at least one additional agent, preferably wherein the additional agent comprises at least one CD70-targeting agent.

The present disclosure also provides methods of identifying if a subject having AML will be responsive to an AML treatment comprising the administration of a combination of at least one BCL-2 inhibitor and at least one hypomethylating agent, the methods comprising: a) determining the mutational status of PTPN11 in a plurality of cells in a sample from the subject; and b) identifying that the subject will not be responsive to the AML treatment when the presence of at least one mutation in PTPN11 is identified; or identifying that the subject will be responsive to the AML treatment when no mutations in PTPN11 are identified.

The present disclosure also provides methods of identifying if a subject having AML will be responsive to an AML treatment comprising the administration of a combination of at least one BCL-2 inhibitor and at least one hypomethylating agent, the methods comprising: a) determining the mutational status of PTPN11 in a plurality of cells in a sample from the subject; b) determining the number and/or percentage of cells in the plurality of cells that exhibit at least one PTPN11 mutation based on step (a); c) comparing the number and/or percentage from step (b) to a predetermined cutoff value; and d) identifying that the subject will not be responsive to the AML treatment when the number and/or percentage of cells that exhibit at least one mutation in PTPN11 is equal to or greater than the predetermined cutoff value; or identifying that the subject will be responsive to the AML treatment when the number and/or percentage of cells that exhibit at least one mutation in PTPN11 is less than the predetermined cutoff value.

In some aspects, the preceding methods can further comprise providing a treatment recommendation to a subject identified as a subject that will not be responsive to treatment with a combination of at least one BCL-2 inhibitor and at least one hypomethylating agent, wherein the treatment recommendation comprises recommending administering at least one CD70-targeting agent, optionally wherein the treatment recommendation further comprises recommending administering the at least one CD70-targeting agent in combination with at least one BCL-2 inhibitor and at least one hypomethylating agent; and/or

In some aspects, the preceding methods can further comprise administering to a subject identified as a subject that will not be responsive to treatment with a combination of at least one BCL-2 inhibitor and at least one hypomethylating agent a treatment comprising at least one CD70-targeting agent, optionally in combination with at least one BCL-2 inhibitor and at least one hypomethylating agent.

The present disclosure provides methods of treating AML in a subject in need thereof, the methods comprising administering at least one CD70-targeting agent to the subject, wherein the subject has at least one AML cell that exhibits a mutation in PTPN11. The present disclosure provides methods of treating AML in a subject in need thereof, the methods comprising administering at least one CD70-targeting agent to the subject, wherein the subject has a number and/or percentage of AML cells that exhibit a mutation in PTPN11 that is equal to or greater than a predetermined cutoff value. In some aspects, the preceding methods can further comprise administering to the subject a combination of at least one BCL-2 inhibitor and at least one hypomethylating agent.

### Treatments

In some aspects of the preceding methods, the first treatment can further comprise a combination of at least one BCL-2 inhibitor and at least one hypomethylating agent.

In some aspects of the preceding methods, the second treatment can further comprise a combination of at least one BCL-2 inhibitor and at least one hypomethylating agent.

In some aspects of the preceding methods, the AML treatment can further comprise the administration of a combination of at least one BCL-2 inhibitor and at least one hypomethylating agent.

### Biomarker Detection

The expression of the biomarkers described above, or any other biomarker describe herein, can be accomplished using any suitable method known in the art by the skilled artisan. Such methods include, but are not limited to, cytogenic analysis, routine karyotyping, fluorescent in situ hybridization (FISH), comparative genomic hybridization (CGH), array comparative genomic hybridization (aCGH), PCR, high-throughput sequencing, next generation sequencing (NGS), Northern Blot, reverse transcription PCR (RT-PCR), real-time PCR (qPCR), quantitative PCR, qRT-PCR, flow cytometry, mass spectrometry, microarray analysis, digital droplet PCR, Western Blot. Cellular Indexing of Transcriptomes and Epitopes by Sequencing (CITE-SEQ), any single-cell omics technology, or any combination thereof.

### Treatment Recommendations

The methods described herein can further comprise a step of providing a treatment recommendation to a clinician and/or subject. Accordingly, if a subject is identified as a subject who will not be responsive to a treatment comprising a CD70-targeting agent, the method can further comprise providing a treatment recommendation that comprises administering a treatment that does not include a CD70-targeting agent. If a subject is identified as a subject that will be responsive to treatment with a CD70-targeting agent, then the method can further comprise providing a treatment recommendation comprising administering at least one CD70-targeting agent to the subject.

### Hypomethylating Agents

As would be appreciated by the skilled artisan, hypomethylating agents are agents that inhibit DNA methylation. In some aspects of the methods presented herein, a hypomethylating agent can be selected from azacitidine and decitabine. In some aspects of the methods presented herein, a hypomethylating agent can be any hypomethylating agent known in the art.

In some aspects, a hypomethylating agent can be azacitidine:

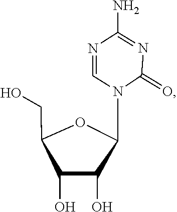

or a pharmaceutically acceptable salt, analog, derivative, salt or ester thereof. As would be appreciated by the skilled artisan, it is understood that azacitidine may be identified by any one of the following names: 5-Azacytidine, Azacytidine, Ladakamycin, 4-Amino-1-β-D-ribofuranosyl-s-triazin-2(1H)-one, U-18496, CC-486 and 4-Amino-1-β-D-ribofuranosyl-1,3,5-triazin-2(1H)-one. As would be appreciated by the skilled artisan, azacitidine may be identified as CAS No. 320-67-2.

In some aspects, a hypomethylating agent can be decitabine:

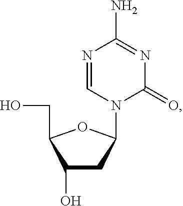

or a pharmaceutically acceptable salt, analog, derivative, salt or ester thereof. As would be appreciated by the skilled artisan, it is understood that decitabine may be identified by any one of the following names: 5-aza-2′-deoxycytidine, 4-Amino-1-(2-deoxy-β-D-erythro-pentofuranosyl)-1,3,5-triazin-2(1H)-one, 5-Aza-2′-deoxycytidine, 5-Azadeoxycytidine, 2-Desoxy-5-azacytidine and 2′-Deoxy-5-azacytidine. As would be appreciated by the skilled artisan, decitabine may be identified as CAS No. 2353-33-5.

### Substitution of Hypomethylating Agent

In any of the methods described herein, a hypomethylating agent can be replaced by cytarabine:

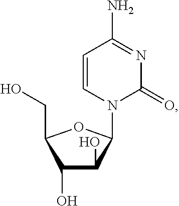

or a pharmaceutically acceptable salt, analog, derivative, salt or ester thereof. As would be appreciated by the skilled artisan, it is understood that cytarabine may be identified by any one of the following names: 4-amino-1-[(2R,3S,4S,5R)-3,4-dihydroxy-5-(hydroxymethyl)oxolan-2-yl] pyrimidin-2-one, Aracytidine and cytosine arabinoside. As would be appreciated by the skilled artisan, cytarabine may be identified as CAS No. 147-94-4.

### BCL-2 Inhibitors

In some aspects of the methods presented herein, a BCL-2 inhibitor can be selected from venetoclax and navitoclax. In some aspects of the methods presented herein, a BCL-2 inhibitor can be any BCL-2 inhibitor known in the art.

In some aspects, a BCL-2 inhibitor can be venetoclax:

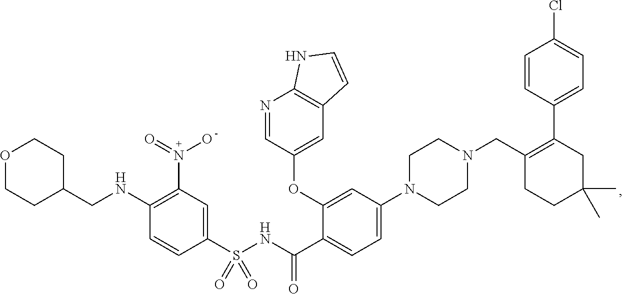

or a pharmaceutically acceptable salt, analog, derivative, salt or ester thereof. As would be appreciated by the skilled artisan, it is understood that venetoclax may be identified by any one of the following names: GDC-0199, ABT-199, RG-7601, 4-(4-{[2-(4-Chlorophenyl)-4,4-dimethyl-1-cyclohexen-1-yl]methyl}-1-piperazinyl)-N-({3-nitro-4-[(tetrahydro-2H-pyran-4-ylmethyl)amino]phenyl}sulfonyl)-2-(1H-pyrrolo[2,3-b]pyridin-5-yloxy)benzamide, Venclexta and Venclyxto. As would be appreciated by the skilled artisan, venetoclax may be identified as CAS No. 1257044-40-8.

In some aspects, a BCL-2 inhibitor can be navitoclax:

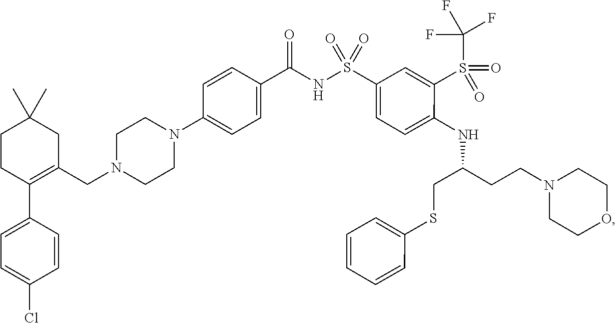 [SMILES: CC1(C)CCC(c2ccc(Cl)cc2)=C(CN2CCN(c3ccc(C(=O)NS(=O)(=O)c4ccc(N[C@H](CCN5CCOCC5)CSc5ccccc5)c(S(=O)(=O)C(F)(F)F)c4)cc3)CC2)C1]

or a pharmaceutically acceptable salt, analog, derivative, salt or ester thereof. As would be appreciated by the skilled artisan, it is understood that navitoclax may be identified by any one of the following names: ABT263, ABT-263 and 4-(4-([2-(4-Chlorophenyl)-5,5-dimethylcyclohex-1-en-1-yl]methyl)piperazin-1-yl)-N-(4-{[(2R)-4-(morpholin-4-yl)-1-(phenylsulfanyl)butan-2-yl]amino}-3-(trifluoromethancsulfonyl)benzene-1-sulfonyl)benzamide. As would be appreciated by the skilled artisan, navitoclax may be identified as CAS No. 923564-51-6.

In some aspects, a BCL-2 inhibitor can be BGB-11417.

In some aspects, a BCL-2 inhibitor can be ZN-d5.

### CD70-Targeting Agent

In some aspects, CD70-targeting agent can be at least one of: i) an anti-CD70 antibody; ii) an anti-CD70 immunotherapy, preferably wherein the immunotherapy comprises CAR-T and/or NK Cells that are directed specifically at CD70; iii) an agent that blocks CD70 signaling, preferably wherein the agent that blocks CD70 signaling prevents binding of CD27 and CD70.

In some aspects, a CD70-targeting agent can be cusatuzumab.

In some aspects of the methods of the present disclosure, immunotherapy can comprise administering a therapeutically effective amount of at least one antibody, at least one checkpoint inhibitor, at least one chimeric antigen receptor-modified T-Cell (CAR-T cell), or any combination thereof. Immunotherapy can comprise adoptive cell transfer therapy.

In some aspects, an immunotherapy can be an immunotherapy that specifically targets CD70. Accordingly, a non-limiting example of an immunotherapy that specifically targets at least one monocytic antigen can be a CAR-T cell that comprises a chimeric antigen receptor comprising an antigen binding domain that binds to CD70.

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity. An antibody that binds to a target refers to an antibody that is capable of binding the target with sufficient affinity such that the antibody is useful as a diagnostic and/or therapeutic agent in targeting the target. In one embodiment, the extent of binding of an anti-target antibody to an unrelated, non-target protein is less than about 10% of the binding of the antibody to target as measured, e.g., by a radioimmunoassay (RIA) or biacore assay. In certain embodiments, an antibody that binds to a target has a dissociation constant (Kd) of <1 μM, <100 nM, <10 nM, <1 nM, <0.1 nM, <0.01 nM, or <0.001 nM (e.g. 10⁸ M or less, e.g. from 10⁸ M to 10¹³ M. e.g., from 10⁹ M to 10¹³ M). In certain embodiments, an anti-target antibody binds to an epitope of a target that is conserved among different species.

A “blocking antibody” or an “antagonist antibody” is one that partially or fully blocks, inhibits, interferes, or neutralizes a normal biological activity of the antigen it binds. For example, an antagonist antibody may block signaling through an immune cell receptor (e.g., a T cell receptor) so as to restore a functional response by T cells (e.g., proliferation, cytokine production, target cell killing) from a dysfunctional state to antigen stimulation.

An “agonist antibody” or “activating antibody” is one that mimics, promotes, stimulates, or enhances a normal biological activity of the antigen it binds. Agonist antibodies can also enhance or initiate signaling by the antigen to which it binds. In some embodiments, agonist antibodies cause or activate signaling without the presence of the natural ligand. For example, an agonist antibody may increase memory T cell proliferation, increase cytokine production by memory T cells, inhibit regulatory T cell function, and/or inhibit regulatory T cell suppression of effector T cell function, such as effector T cell proliferation and/or cytokine production.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)2; diabodies; linear antibodies; single-chain antibody molecules (e.g. scFv); and multispecific antibodies formed from antibody fragments.

CAR-T cells are T cells that are genetically modified to stably express at least one chimeric antigen receptor (CAR). A CAR can comprise an extracellular domain, transmembrane domain and a cytoplasmic domain. A CAR can comprise an antigen binding domain. An antigen binding domain can be located in an extracellular domain. In some aspects of the methods of the present disclosure, the antigen binding domain binds to at least one AML cell surface protein. In some aspects of the methods of the present disclosure, the antigen binding domain binds to CD70. A CAR can also comprise an extracellular spacer (hinge) domain. An extracellular spacer can be located in an extracellular domain. A CAR can comprise a signaling domain. A signaling domain can be a T-cell activation domain. A signaling domain can be located in a cytoplasmic domain. A CAR can comprise at least one costimulatory domain. A CAR can comprise at least two costimulatory domains. A CAR can comprise at least three costimulatory domains. A costimulatory domain can be located in a cytoplasmic domain.

In some aspects of the methods of the present disclosure CAR-T cells can be autologous with respect to a subject. In some aspects, CAR-T cells can be allogeneic with respect to a subject.

In some aspects of the methods of the present disclosure, CAR-T cells may be administered either alone, or as a pharmaceutical composition in combination with diluents and/or with other components such as IL-2 or other cytokines or cell populations. Briefly, pharmaceutical compositions can comprise a plurality of CAR-T cells in combination with one or more pharmaceutically or physiologically acceptable carriers, diluents or excipients.

Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. CAR-T cells and related compositions can be administered to a subject intravenously.

A CAR-T cell can comprise a chimeric antigen receptor. A chimeric antigen receptor can comprise an antigen binding domain. An antigen binding domain can bind to CD70.

### Subjects

As used herein, the term “subject” includes human and non-human animals, as well as cell lines, cell cultures, tissues, and organs. In some aspects, the subject is a mammal. The mammal can be e.g., a human or appropriate non-human mammal, such as primate, mouse, rat, dog, cat, cow, horse, goat, camel, sheep or a pig. The subject can also be a bird or fowl. In some aspects, the subject is a human.

As used herein, the term “subject in need thereof” refers, both of which refer to a subject having a disease or having an increased risk of developing the disease. A “subject” includes a mammal. The mammal can be e.g., a human or appropriate non-human mammal, such as primate, mouse, rat, dog, cat, cow, horse, goat, camel, sheep or a pig. The subject can also be a bird or fowl. In some aspects, the mammal is a human. A subject in need thereof can be one who has been previously diagnosed or identified as having a disease or disorder disclosed herein. A subject in need thereof can also be one who is suffering from a disease or disorder disclosed herein. Alternatively, a subject in need thereof can be one who has an increased risk of developing such disease or disorder relative to the population at large (i.e., a subject who is predisposed to developing such disorder relative to the population at large). A subject in need thereof can have a refractory or resistant a disease or disorder disclosed herein (i.e., a disease or disorder disclosed herein that does not respond or has not yet responded to treatment). The subject may be resistant at start of treatment or may become resistant during treatment. In some aspects, the subject in need thereof received and failed all known effective therapies for a disease or disorder disclosed herein. In some aspects, the subject in need thereof received at least one prior therapy.

In some aspects of the methods of the present disclosure, the subject is a human.

In some aspects of the methods of the present disclosure, the subject is a subject having AML who has not received any treatment for AML.

In some aspects of the methods of the present disclosure, the subject is a subject having AML who has received previously received at least one AML treatment. In some aspects, the at least one treatment comprises a combination of at least one BCL-2 inhibitor and at least one hypomethylating agent.

In some aspects of the methods of the present disclosure, the subject is a subject having AML that has not received a stem cell transplant; or

In some aspects of the methods of the present disclosure, the subject is a subject having AML that has previously received at least one stem cell transplant.

### Samples

In some aspects of the methods of the present disclosure, a sample can comprise blood, a bone marrow biopsy, a bone marrow aspirate, a biopsy of a chloroma, a tissue biopsy, cerebrospinal fluid or any combination thereof.

In some aspects, a sample can be a bone marrow biopsy.

In some aspects, a sample can be a bone marrow aspirate.

In some aspects, a sample can be a biopsy of a chloroma.

In any of the methods described herein, a plurality of cells from a sample can comprise acute myeloid leukemia cells. In some aspects, acute myeloid leukemia cells comprise acute myeloid leukemia blast cells, leukemia stem cells, or any combination thereof.

### General Definitions

As used herein, the expressions “one or more of A. B, or C,” “one or more A, B, or C,” “one or more of A, B, and C,” “one or more A, B, and C,” “selected from the group consisting of A, B, and C”, “selected from A. B, and C”, and the like are used interchangeably and all refer to a selection from a group consisting of A. B, and/or C, i.e., one or more As, one or more Bs, one or more Cs, or any combination thereof, unless indicated otherwise.

It is to be understood that, unless otherwise stated, any description of a method of treatment includes use of the agents to provide such treatment as is described herein. It is to be further understood, unless otherwise stated, any description of a method of treatment includes use of the agents to prepare a medicament to treat such condition. The treatment includes treatment of human or non-human animals including rodents and other disease models used herein.

As used herein, the term “treating” or “treat” describes the management and care of a patient for the purpose of combating a disease, condition, or disorder and includes the administration of a compound of the present disclosure, or a pharmaceutically acceptable salt, polymorph or solvate thereof, to alleviate the symptoms or complications of a disease, condition or disorder, or to eliminate the disease, condition or disorder. The term “treat” can also include treatment of a cell in vitro or an animal model.

It is to be understood that a compound of the present disclosure, or a pharmaceutically acceptable salt, polymorph or solvate thereof, can or may also be used to prevent a relevant disease, condition or disorder, or used to identify suitable candidates for such purposes.

As used herein, the term “preventing,” “prevent,” or “protecting against” describes reducing or eliminating the onset of the symptoms or complications of such disease, condition or disorder.

As used herein, the term “pharmaceutically acceptable” refers to those compounds, anions, cations, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

It is understood that the agents described herein can be administered to a subject in at least one therapeutically effective amount. As used herein, the term “therapeutically effective amount”, refers to an amount of a pharmaceutical agent to treat, ameliorate, or prevent an identified disease or condition, or to exhibit a detectable therapeutic or inhibitory effect. The effect can be detected by any assay method known in the art. The precise effective amount for a subject will depend upon the subject's body weight, size, and health; the nature and extent of the condition; and the therapeutic or combination of therapeutics selected for administration. Therapeutically effective amounts for a given situation can be determined by routine experimentation that is within the skill and judgment of the clinician. A clinician can also determine the therapeutically effective amounts of the agents described herein using established dosing and administration protocols for the agents described herein.

It is to be understood that, for any agent, the therapeutically effective amount can be estimated initially either in cell culture assays. e.g., of neoplastic cells, or in animal models, usually rats, mice, rabbits, dogs, or pigs. The animal model may also be used to determine the appropriate concentration range and route of administration. Such information can then be used to determine useful doses and routes for administration in humans. Therapeutic/prophylactic efficacy and toxicity may be determined by standard pharmaceutical procedures in cell cultures or experimental animals. e.g., ED₅₀ (the dose therapeutically effective in 50% of the population) and LD₅₀ (the dose lethal to 50% of the population). The dose ratio between toxic and therapeutic effects is the therapeutic index, and it can be expressed as the ratio, LD₅₀/ED₅₀. Pharmaceutical compositions that exhibit large therapeutic indices are preferred. The dosage may vary within this range depending upon the dosage form employed, sensitivity of the patient, and the route of administration.

Dosage and administration are adjusted to provide sufficient levels of the active agent(s) or to maintain the desired effect. Factors which may be taken into account include the severity of the disease state, general health of the subject, age, weight, and gender of the subject, diet, time and frequency of administration, drug combination(s), reaction sensitivities, and tolerance/response to therapy. Long-acting pharmaceutical compositions may be administered every 3 to 4 days, every week, or once every two weeks depending on half-life and clearance rate of the particular formulation.

As used herein, the term “pharmaceutically acceptable salts” refer to derivatives of the agents described herein wherein the agent is modified by making acid or base salts thereof. Examples of pharmaceutically acceptable salts include, but are not limited to, mineral or organic acid salts of basic residues such as amines, alkali or organic salts of acidic residues such as carboxylic acids, and the like. The pharmaceutically acceptable salts include the conventional non-toxic salts or the quaternary ammonium salts of the parent agent formed, for example, from non-toxic inorganic or organic acids. For example, such conventional non-toxic salts include, but are not limited to, those derived from inorganic and organic acids selected from 2-acetoxybenzoic, 2-hydroxyethane sulfonic, acetic, ascorbic, benzene sulfonic, benzoic, bicarbonic, carbonic, citric, edetic, ethane disulfonic, 1,2-ethane sulfonic, fumaric, glucoheptonic, gluconic, glutamic, glycolic, glycollyarsanilic, hexylresorcinic, hydrabamic, hydrobromic, hydrochloric, hydroiodic, hydroxymaleic, hydroxynaphthoic, isethionic, lactic, lactobionic, lauryl sulfonic, maleic, malic, mandelic, methane sulfonic, napsylic, nitric, oxalic, pamoic, pantothenic, phenylacetic, phosphoric, polygalacturonic, propionic, salicylic, stearic, subacetic, succinic, sulfamic, sulfanilic, sulfuric, tannic, tartaric, toluene sulfonic, and the commonly occurring amine acids, e.g., glycine, alanine, phenylalanine, arginine, etc.

In some aspects, the pharmaceutically acceptable salt is a sodium salt, a potassium salt, a calcium salt, a magnesium salt, a diethylamine salt, a choline salt, a meglumine salt, a benzathine salt, a tromethamine salt, an ammonia salt, an arginine salt, or a lysine salt.

Other examples of pharmaceutically acceptable salts include hexanoic acid, cyclopentane propionic acid, pyruvic acid, malonic acid, 3-(4-hydroxybenzoyl)benzoic acid, cinnamic acid, 4-chlorobenzenesulfonic acid, 2-naphthalenesulfonic acid, 4-toluenesulfonic acid, camphorsulfonic acid, 4-methylbicyclo-[2.2.2]-oct-2-ene-1-carboxylic acid, 3-phenylpropionic acid, trimethylacetic acid, tertiary butylacetic acid, muconic acid, and the like. The present disclosure also encompasses salts formed when an acidic proton present in the parent agent either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like. In the salt form, it is understood that the ratio of the agent to the cation or anion of the salt can be 1:1, or any ratio other than 1:1, e.g., 3:1, 2:1, 1:2, or 1:3.

It is to be understood that all references to pharmaceutically acceptable salts include solvent addition forms (solvates) or crystal forms (polymorphs) as defined herein, of the same salt.

The term “refractory” as used herein, is used in its broadest sense to refer to instances in which the disease present in a subject does not respond to a particular therapy. i.e. the therapy provides no or decreased clinical benefit to that particular subject.

In some aspects, identifying that a subject will be responsive to an AML treatment comprises identifying that the subject will have a durable remission after receiving the AML treatment.

In some aspects, identifying that a subject will not be responsive to an AML treatment comprises identifying that the subject will be refractory to the AML treatment and/or that the subject will suffer a relapse after receiving the AML treatment.

As used herein, the term “combination therapy” or “co-therapy” includes the administration of an agent disclosed herein, or a pharmaceutically acceptable salt, polymorph or solvate thereof, and at least a second agent as part of a specific treatment regimen intended to provide the beneficial effect from the co-action of these therapeutic agents. The beneficial effect of the combination includes, but is not limited to, pharmacokinetic or pharmacodynamic co-action resulting from the combination of therapeutic agents.

As used herein, the term “temporal proximity” refers to that administration of one therapeutic agent occurs within a time period before or after the administration of another therapeutic agent, such that the therapeutic effect of the one therapeutic agent overlaps with the therapeutic effect of the other therapeutic agent. In some embodiments, the therapeutic effect of the one therapeutic agent completely overlaps with the therapeutic effect of the other therapeutic agent. In some embodiments, “temporal proximity” means that administration of one therapeutic agent occurs within a time period before or after the administration of another therapeutic agent, such that there is a synergistic effect between the one therapeutic agent and the other therapeutic agent. “Temporal proximity” may vary according to various factors, including but not limited to, the age, gender, weight, genetic background, medical condition, disease history, and treatment history of the subject to which the therapeutic agents are to be administered; the disease or condition to be treated or ameliorated; the therapeutic outcome to be achieved; the dosage, dosing frequency, and dosing duration of the therapeutic agents; the pharmacokinetics and pharmacodynamics of the therapeutic agents; and the route(s) through which the therapeutic agents are administered. In some embodiments, “temporal proximity” means within 15 minutes, within 30 minutes, within an hour, within two hours, within four hours, within six hours, within eight hours, within 12 hours, within 18 hours, within 24 hours, within 36 hours, within 2 days, within 3 days, within 4 days, within 5 days, within 6 days, within a week, within 2 weeks, within 3 weeks, within 4 weeks, with 6 weeks, or within 8 weeks. In some embodiments, multiple administration of one therapeutic agent can occur in temporal proximity to a single administration of another therapeutic agent. In some embodiments, temporal proximity may change during a treatment cycle or within a dosing regimen.

### EXEMPLARY EMBODIMENTS

1. A method of treating acute myeloid leukemia (AML) in a subject, the method comprising:
a) measuring the LDH level of the subject:
b) comparing the subject's LDH level to a predetermined cutoff value; and
c) administering to the subject a first treatment when the subject's LDH level is equal to or greater than a predetermined cutoff value, wherein the first treatment comprises at least one CD70-targeting agent; or administering to the subject a second treatment when the subject's LDH level is less than the predetermined cutoff value, wherein the second treatment does not comprise a CD70-targeting agent.
2. A method of identifying if a subject having AML will be responsive to an AML treatment comprising the administration of at least one CD70-targeting agent, the method comprising:
a) measuring the LDH level of the subject;
b) comparing the subject's LDH level to a predetermined cutoff value; and
c) identifying that the subject will be responsive to the AML treatment when the subject's LDH level is equal to or greater than the predetermined cutoff value.
3. A method of treating AML in a subject, the method comprising:
a) measuring the expression of at least CD56 in a plurality of cells in a sample from the subject;
b) identifying the presence of at least one CD56+ cell based on the expression measured in step (a); and
c) administering to the subject a first treatment when CD56+ cells are identified, wherein the first treatment comprises at least one CD70-targeting agent; or administering to the subject a second treatment when no CD56+ cells are identified, wherein the second treatment does not comprise a CD70-targeting agent.
4. A method of identifying if a subject having AML will be responsive to an AML treatment comprising the administration of at least one CD70-targeting agent, the method comprising:
a) measuring the expression of at least CD56 in a plurality of cells in a sample from the subject;
b) identifying the presence of at least one CD56+ cell based on the expression measured in step (a); and
c) identifying that the subject will be responsive to the AML treatment when CD56+ cells are identified.
5. A method of treating AML in a subject, the method comprising:
a) determining the mutational status of at least one of BRAF, JAK2, KRAS, SMC1A, STAG2, TET2, TP53, U2AF1 and PTPN11 in a plurality of cells in a sample from the subject; and
b) administering to the subject a first treatment when at least one mutation in at least one of BRAF, JAK2. KRAS. SMC1A, STAG2, TET2, TP53. U2AF1 and PTPN11 are identified, wherein the first treatment comprises at least one CD70-targeting agent; or
administering to the subject a second treatment when no mutations in BRAF, JAK2, KRAS, SMC1A, STAG2, TET2, TP53, U2AF1 and PTPN11 are identified, wherein the second treatment does not comprise a CD70-targeting agent.
6. A method of identifying if a subject having AML will be responsive to an AML treatment comprising the administration of at least one CD70-targeting agent, the method comprising:
a) determining the mutational status of at least one of BRAF, JAK2, KRAS, SMC1A, STAG2, TET2, TP53, U2AF1 and PTPN11 in a plurality of cells in a sample from the subject; and
b) identifying that the subject will be responsive to the AML treatment when the presence of at least one mutation in at least one of BRAF, JAK2, KRAS, SMC1A, STAG2, TET2, TP53, U2AF1 and PTPN11 is identified.
7. A method of treating AML in a subject, the method comprising:
a) determining the mutational status of at least one of NPM1, IDH1, IDH2, RUNX1, SF3B1, GATA2, BCOR, BCORL1 and EZH2 in a plurality of cells in a sample from the subject; and
b) administering to the subject a first treatment when at least one mutation in at least one of NPM1, IDH1, IDH2, RUNX1, SF3B1, GATA2, BCOR, BCORL1 and EZH2 are identified, wherein the first treatment does not comprise at least one CD70-targeting agent; or administering to the subject a second treatment when no mutations in NPM1, IDH1, IDH2, RUNX1, SF3B1, GATA2, BCOR, BCORL1 and EZH2 are identified, wherein the second treatment comprises at least one CD70-targeting agent.
8. A method of identifying if a subject having AML will be responsive to an AML treatment comprising the administration of at least one CD70-targeting agent, the method comprising:
a) determining the mutational status of at least one of NPM1, IDH1, IDH2, RUNX1, SF3B1, GATA2, BCOR, BCORL1 and EZH2 in a plurality of cells in a sample from the subject; and b) identifying that the subject will not be responsive to the AML treatment when the presence of at least one mutation in at least one of NPM1, IDH1, IDH2, RUNX1, SF3B1, GATA2, BCOR, BCORL1 and EZH2 is identified.
9. A method of treating AML in a subject, the method comprising:
a) determining the mutational status of at least one of FLT3 in a plurality of cells in a sample from the subject; and
b) administering to the subject a first treatment when the FLT3-ITD mutation is identified, wherein the first treatment comprises at least one CD70-targeting agent; or administering to the subject a second treatment when no FLT3-ITD mutation is identified, wherein the second treatment does not comprise a CD70-targeting agent.
10. A method of identifying if a subject having AML will be responsive to an AML treatment comprising the administration of at least one CD70-targeting agent, the method comprising:
a) determining the mutational status of FLT3 in a plurality of cells in a sample from the subject; and
b) identifying that the subject will be responsive to the AML treatment when the presence of the FLT3-ITD mutation is identified.
11. A method of treating AML in a subject, the method comprising:
a) determining the cytogenetic status of a plurality of cells in a sample from the subject; and
b) administering to the subject a first treatment when at least one cell is identified as having at least one of an inv(3) phenotype, a Del7q phenotype, a 17⁻ phenotype, a 7 centromere phenotype, and an EGR1⁻ phenotype wherein the first treatment comprises at least one CD70-targeting agent; or administering to the subject a second treatment when no cells having an inv(3) are identified, wherein the second treatment does not comprise a CD70-targeting agent.
12. A method of identifying if a subject having AML will be responsive to an AML treatment comprising the administration of at least one CD70-targeting agent, the method comprising:
a) determining the cytogenetic status of a plurality of cells in a sample from the subject, and
b) identifying that the subject will be responsive to the AML treatment when the presence of at least cell exhibiting at least one of an inv(3) phenotype, a Del7q phenotype, a 17⁻ phenotype, a 7 centromere phenotype, and an EGR1⁻ phenotype is identified.
13. A method of treating AML in a subject, the method comprising:
a) determining if the subject has monocytic AML; and
b) administering to the subject a first treatment when the subject is identified as having monocytic AML, wherein the first treatment comprises at least one CD70-targeting agent; or administering to the subject a second treatment when the subject is identified as not having monocytic AML, wherein the second treatment does not comprise a CD70-targeting agent.
14. A method of identifying if a subject having AML will be responsive to an AML treatment comprising the administration of at least one CD70-targeting agent, the method comprising:
a) determining if the subject has monocytic AML; and
b) identifying that the subject will be responsive to the AML treatment when the subject is identified as having monocytic AML.
15. The method of any one of the preceding embodiments, wherein the first treatment further comprises a combination of at least one BCL-2 inhibitor and at least one hypomethylating agent.
16. The method of any one of the preceding embodiments, wherein the second treatment further comprises a combination of at least one BCL-2 inhibitor and at least one hypomethylating agent.
17. The method of any one of embodiments 2, 4, 6, 10, 12 and 14, wherein the AML treatment further comprises the administration of a combination of at least one BCL-2 inhibitor and at least one hypomethylating agent.
18. The method of any one of embodiments 2, 4, 6, 10, 12 and 14, wherein the method further comprises:
a) providing a treatment recommendation to the subject, wherein the treatment recommendation comprises recommending administering at least one CD70-targeting agent, optionally wherein the treatment recommendation further comprises recommending administering the at least one CD70-targeting agent in combination with at least one BCL-2 inhibitor and at least one hypomethylating agent; and/or b) administering to the subject a treatment comprising at least one CD70-targeting agent, optionally in combination with at least one BCL-2 inhibitor and at least one hypomethylating agent.
19. The method of any one of the preceding embodiments, wherein the at least one CD70-targeting agent comprises:
i) an anti-CD70 antibody, preferably wherein the anti-CD70 antibody is cusatuzumab;
ii) an anti-CD70 immunotherapy, preferably wherein the immunotherapy comprises CAR-T and/or NK Cells that are directed specifically at CD70;
iii) an agent that blocks CD70 signaling, preferably wherein the agent that blocks CD70 signaling prevents binding of CD27 and CD70.
20. The method of any one of the preceding embodiments, wherein the at least one hypomethylating agent is selected from azacitidine and decitabine.
21. The method of any one of the preceding embodiments, wherein the at least one BCL-2 inhibitor is selected from venetoclax and navitoclax.
22. The method of any one of the preceding embodiments, wherein step (a) comprises performing cytogenic analysis, routine karyotyping, fluorescent in situ hybridization (FISH), comparative genomic hybridization (CGH), array comparative genomic hybridization (aCGH), PCR, high-throughput sequencing, next generation sequencing (NGS), Northern Blot, reverse transcription PCR (RT-PCR), real-time PCR (qPCR), quantitative PCR, qRT-PCR, flow cytometry, mass spectrometry, microarray analysis, digital droplet PCR, Western Blot, Cellular Indexing of Transcriptomes and Epitopes by Sequencing (CITE-SEQ), any single-cell omics technology or any combination thereof.
23. The method of any one of the preceding embodiments, wherein the subject is:
i) a subject having AML who has not received any treatment for AML; or
ii) a subject having AML who has received previously received at least one AML treatment, preferably wherein the at least one AML treatment comprises a combination of at least one BCL-2 inhibitor and at least one hypomethylating agent.
24. The method of any one of the preceding embodiments, wherein the subject is:
i) a subject that has not received a stem cell transplant; or
ii) a subject that has previously received at least one stem cell transplant.
25. The method of any one of the preceding embodiments, wherein identifying that a subject will be responsive to the AML treatment comprises identifying that the subject will have a durable remission after receiving the AML treatment.
26. The method of any one of the preceding embodiments, wherein the biological sample comprises blood, a bone marrow biopsy, a bone marrow aspirate, a biopsy of a chloroma, a tissue biopsy, cerebrospinal fluid or any combination thereof.
27. The method of any of the preceding embodiments, wherein the plurality of cells comprises acute myeloid leukemia cells.
28. The method of embodiment 24, wherein the acute myeloid leukemia cells comprise acute myeloid leukemia blast cells, leukemia stem cells, or any combination thereof.
29. A method of treating acute myeloid leukemia (AML) in a subject, the method comprising:
a) measuring the expression of at least CD2 in a plurality of cells in a sample from the subject:
b) identifying the presence of at least one CD2+ cell based on the expression measured in step (a); and
c) administering to the subject a first treatment when no CD2+ cells are identified, wherein the first treatment comprises at least one CD70-targeting agent; or
administering to the subject a second treatment when CD2+ cells are identified, wherein the second treatment does not comprise a CD70-targeting agent.
30. A method of identifying if a subject having AML will be responsive to an AML treatment comprising the administration of at least one CD70-targeting agent, the method comprising:
a) measuring the expression of at least CD2 in a plurality of cells in a sample from the subject;
b) identifying the presence of at least one CD2+ cell based on the expression measured in step (a); and
c) identifying that the subject will not be responsive to the AML treatment when the presence of at least one CD2+ cell is identified; or identifying that the subject will be responsive to the AML treatment when no CD2+ cells are identified.
31. A method of treating AML in a subject, the method comprising:
a) determining the mutational status of NF1 in a plurality of cells in a sample from the subject; and
b) administering to the subject a first treatment when no mutations in NF1 are identified, wherein the first treatment comprises at least one CD70-targeting agent; or administering to the subject a second treatment when at least one mutation in NF1 is identified, wherein the second treatment does not comprise a CD70-targeting agent.
32. A method of identifying if a subject having AML will be responsive to an AML treatment comprising the administration of at least one CD70-targeting agent, the method comprising:
a) determining the mutational status of NF1 in a plurality of cells in a sample from the subject; and
b) identifying that the subject will not be responsive to the AML treatment when the presence of at least one mutation in NF1 is identified; or identifying that the subject will be responsive to the AML treatment when no mutations in NF1 are identified.
33. A method of treating AML in a subject, the method comprising:
a) determining the chromosome 7 status in a plurality of cells in a sample from the subject; and
b) administering to the subject a first treatment when no chromosome 7 abnormalities are identified, wherein the first treatment comprises at least one CD70-targeting agent; or administering to the subject a second treatment when at least one chromosome 7 abnormality is identified, wherein the second treatment does not comprise a CD70-targeting agent.
34. A method of identifying if a subject having AML will be responsive to an AML treatment comprising the administration of at least one CD70-targeting agent, the method comprising:
a) determining the chromosome 7 status in a plurality of cells in a sample from the subject; and
b) identifying that the subject will not be responsive to the AML treatment when the presence of at least one chromosome 7 abnormality is identified; or identifying that the subject will be responsive to the AML treatment when no chromosome 7 abnormalities are identified.
35. A method of treating AML in a subject, the method comprising:
a) determining the mutational status of TP53 in a plurality of cells in a sample from the subject; and
b) administering to the subject a first treatment when no mutations in TP53 are identified, wherein the first treatment comprises at least one CD70-targeting agent; or administering to the subject a second treatment when at least one mutation in TP53 is identified, wherein the second treatment does not comprise a CD70-targeting agent.
36. A method of identifying if a subject having AML will be responsive to an AML treatment comprising the administration of at least one CD70-targeting agent, the method comprising:
a) determining the mutational status of TP53 in a plurality of cells in a sample from the subject; and
b) identifying that the subject will not be responsive to the AML treatment when the presence of at least one mutation in TP53 is identified; or identifying that the subject will be responsive to the AML treatment when no mutations in NF1 are identified.
37. A method of treating AML in a subject, the method comprising:
a) measuring the expression of at least CD7 in a plurality of cells in a sample from the subject;
b) identifying the presence of at least one CD7+ cell based on the expression measured in step (a); and
c) administering to the subject a first treatment when no CD7+ cells are identified, wherein the first treatment comprises a combination of at least one BCL-2 inhibitor and at least one hypomethylating agent; or administering to the subject a second treatment when CD7+ cells are identified, wherein the second treatment comprises a combination of at least one BCL-2 inhibitor, at least one hypomethylating agent and at least one additional agent, preferably wherein the additional agent comprises at least one CD70-targeting agent.
38. A method of identifying if a subject having AML will be responsive to an AML treatment comprising the administration of a combination of at least one BCL-2 inhibitor and at least one hypomethylating agent, the method comprising:
a) measuring the expression of at least CD7 in a plurality of cells in a sample from the subject;
b) identifying the presence of at least one CD7+ cell based on the expression measured in step (a); and
c) identifying that the subject will not be responsive to the AML treatment when the presence of at least one CD7+ cell is identified; or
identifying that the subject will be responsive to the AML treatment when no CD7+ cells are identified.
39. A method of treating AML in a subject, the method comprising:
a) determining the mutational status of KRAS in a plurality of cells in a sample from the subject; and
b) administering to the subject a first treatment when no mutations in KRAS are identified, wherein the first treatment comprises a combination of at least one BCL-2 inhibitor and at least one hypomethylating agent; or administering to the subject a second treatment when at least one mutation in KRAS is identified, wherein the second treatment comprises a combination of at least one BCL-2 inhibitor, at least one hypomethylating agent and at least one additional agent, preferably wherein the additional agent comprises at least one CD70-targeting agent.
40. A method of identifying if a subject having AML will be responsive to an AML treatment comprising the administration of a combination of at least one BCL-2 inhibitor and at least one hypomethylating agent, the method comprising:
a) determining the mutational status of KRAS in a plurality of cells in a sample from the subject; and
b) identifying that the subject will not be responsive to the AML treatment when the presence of at least one mutation in KRAS is identified; or identifying that the subject will be responsive to the AML treatment when no mutations in KRAS are identified.
41. A method of treating AML in a subject, the method comprising:
a) determining the mutational status of PTPN1 in a plurality of cells in a sample from the subject; and
b) administering to the subject a first treatment when no mutations in PTPN1 are identified, wherein the first treatment comprises a combination of at least one BCL-2 inhibitor and at least one hypomethylating agent; or administering to the subject a second treatment when at least one mutation in PTPN1 is identified, wherein the second treatment comprises a combination of at least one BCL-2 inhibitor, at least one hypomethylating agent and at least one additional agent, preferably wherein the additional agent comprises at least one CD70-targeting agent.
42. A method of identifying if a subject having AML will be responsive to an AML treatment comprising the administration of a combination of at least one BCL-2 inhibitor and at least one hypomethylating agent, the method comprising:
a) determining the mutational status of PTPN1 in a plurality of cells in a sample from the subject; and
b) identifying that the subject will not be responsive to the AML treatment when the presence of at least one mutation in PTPN1 is identified; or identifying that the subject will be responsive to the AML treatment when no mutations in PTPN1 are identified.
43. The method of embodiment 33 or embodiment 34, wherein the at least one chromosome 7 abnormality comprises chromosome 7 deletion.
44. The method of any one of the preceding embodiments, wherein the first treatment further comprises a combination of at least one BCL-2 inhibitor and at least one hypomethylating agent.
45. The method of any one of the preceding embodiments, wherein the second treatment further comprises a combination of at least one BCL-2 inhibitor and at least one hypomethylating agent.
46. The method of any one of embodiments 30, 32, 34 and 36, wherein the AML treatment further comprises the administration of a combination of at least one BCL-2 inhibitor and at least one hypomethylating agent.
47. The method of any one of embodiments 38, 40 and 42, wherein the method further comprises:
a) providing a treatment recommendation to the subject, wherein the treatment recommendation comprises recommending administering at least one CD70-targeting agent, optionally wherein the treatment recommendation further comprises recommending administering the at least one CD70-targeting agent in combination with at least one BCL-2 inhibitor and at least one hypomethylating agent; and/or
b) administering to the subject a treatment comprising at least one CD70-targeting agent, optionally in combination with at least one BCL-2 inhibitor and at least one hypomethylating agent.
48. The method of any one of the preceding embodiments, wherein the at least one CD70-targeting agent comprises:
i) an anti-CD70 antibody, preferably wherein the anti-CD70 antibody is cusatuzumab;
ii) an anti-CD70 immunotherapy, preferably wherein the immunotherapy comprises CAR-T and/or NK Cells that are directed specifically at CD70;
iii) an agent that blocks CD70 signaling, preferably wherein the agent that blocks CD70 signaling prevents binding of CD27 and CD70.
49. The method of any one of the preceding embodiments, wherein the at least one hypomethylating agent is selected from azacitidine and decitabine.
50. The method of any one of the preceding embodiments, wherein the at least one BCL-2 inhibitor is selected from venetoclax and navitoclax.
51. The method of any one of the preceding embodiments, wherein step (a) comprises performing cytogenic analysis, routine karyotyping, fluorescent in situ hybridization (FISH), comparative genomic hybridization (CGH), array comparative genomic hybridization (aCGH), PCR, high-throughput sequencing, next generation sequencing (NGS), Northern Blot, reverse transcription PCR (RT-PCR), real-time PCR (qPCR), quantitative PCR, qRT-PCR, flow cytometry, mass spectrometry, microarray analysis, digital droplet PCR, Western Blot, Cellular Indexing of Transcriptomes and Epitopes by Sequencing (CITE-SEQ), any single-cell omics technology or any combination thereof.
52. The method of any one of the preceding embodiments, wherein the subject is:
i) a subject having AML who has not received any treatment for AML; or
ii) a subject having AML who has received previously received at least one AML treatment, preferably wherein the at least one AML treatment comprises a combination of at least one BCL-2 inhibitor and at least one hypomethylating agent.
53. The method of any one of the preceding embodiments, wherein the subject is:
i) a subject that has not received a stem cell transplant; or
ii) a subject that has previously received at least one stem cell transplant.
54. The method of any one of the preceding embodiments, wherein identifying that a subject will be responsive to the AML treatment comprises identifying that the subject will have a durable remission after receiving the AML treatment.
55. The method of any one of the preceding embodiments, wherein identifying that a subject will not be responsive to the AML treatment comprises identifying that the subject will be refractory to the AML treatment and/or that the subject will suffer a relapse after receiving the AML treatment.
56. The method of any one of the preceding embodiments, wherein the biological sample comprises blood, a bone marrow biopsy, a bone marrow aspirate, a biopsy of a chloroma, a tissue biopsy, cerebrospinal fluid or any combination thereof.
57. The method of any of the preceding embodiments, wherein the plurality of cells comprises acute myeloid leukemia cells.
58. The method of embodiment 57, wherein the acute myeloid leukemia cells comprise acute myeloid leukemia blast cells, leukemia stem cells, or any combination thereof.

### EXPERIMENTAL EXAMPLES

### Example 1

The following is a non-limiting example of analysis showing the response of AML subjects to treatment with a combination of venetoclax and azacitidine, or a combination of venetoclax, azacitidine and cusatuzumab, based on their LDH levels.

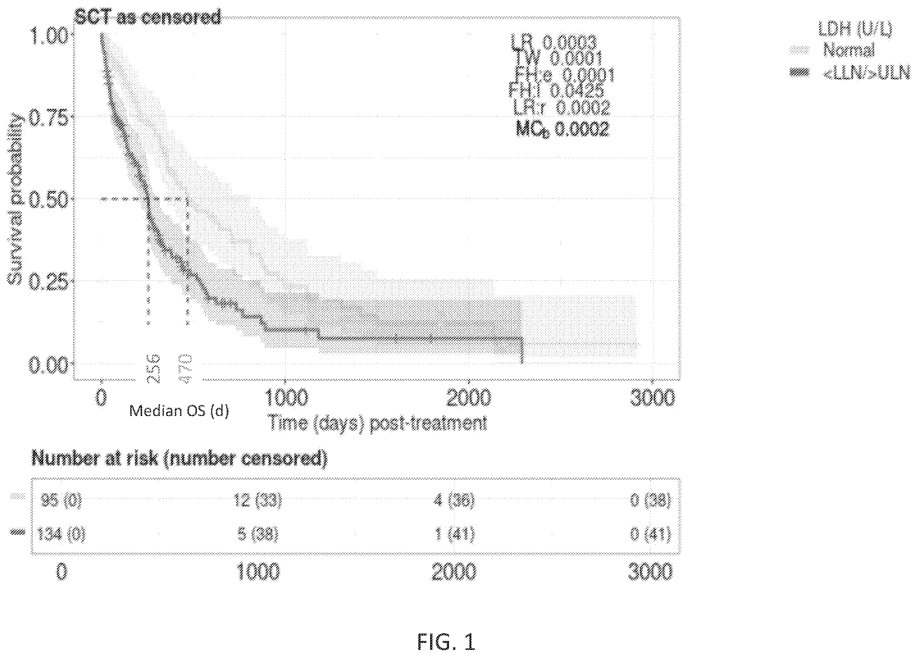

FIG. 1 shows the survival probability of AML subjects exhibiting normal LDH levels or elevated LDH levels after receiving a combination of venetoclax and azacitidine. Elevated LDH levels in FIG. 1 correspond to LDH levels that are greater than the Upper Limit of Normal (ULN) for the laboratory in which the LDH levels were measured.

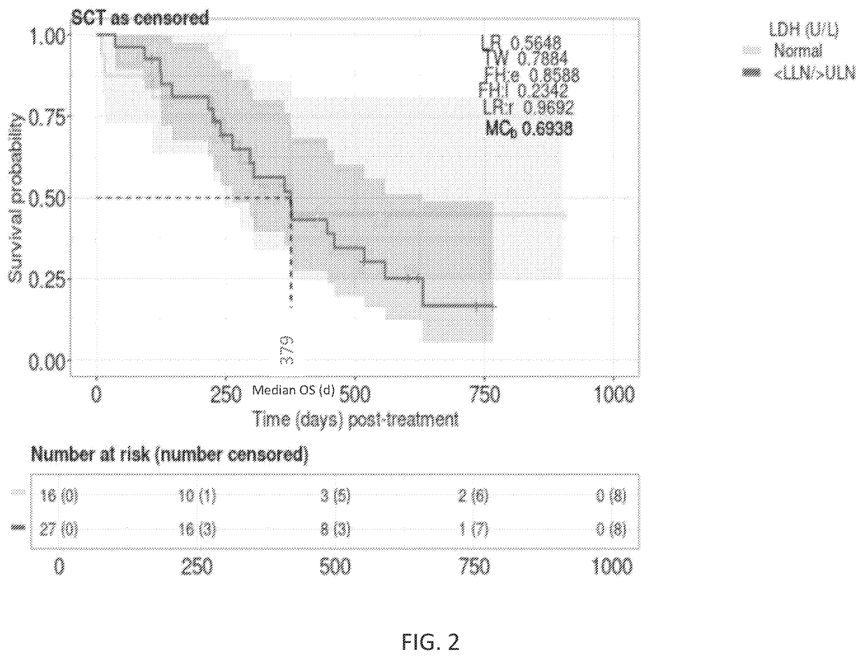

FIG. 2 shows the survival probability of AML subjects exhibiting normal LDH levels or elevated LDH levels after receiving a combination of venetoclax, azacitidine and cusatuzumab. Elevated LDH levels in FIG. 2 correspond to LDH levels that are greater than the Upper Limit of Normal (ULN) for the laboratory in which the LDH levels were measured.

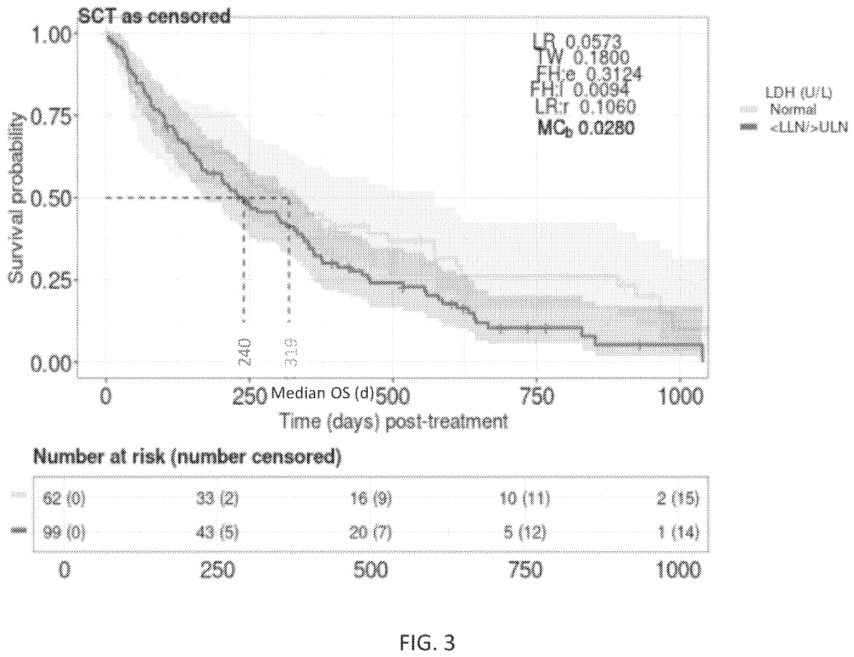

FIG. 3 shows the survival probability of AML subjects exhibiting normal LDH levels or elevated LDH levels after a treatment that includes at least cusatuzumab. Elevated LDH levels in FIG. 3 correspond to LDH levels that are greater than the Upper Limit of Normal (ULN) for the laboratory in which the LDH levels were measured.

Without wishing to be bound by theory, the results presented in FIGS. 1-3 demonstrate that subjects having AML who exhibit increased LDH levels benefit from treatment with a CD-70 target agent.

### Example 2

The following is a non-limiting example of the development of a risk stratification model for AML subjects that can be used to predict their response to treatment with a combination of a BCL-2 inhibitor (e.g. venetoclax) and a hypomethylating agent (e.g. azacitidine). The model allows for the identification of AML biomarkers that can be used to predict the response of a patient to treatment with a combination of a BCL-2 inhibitor (e.g. venetoclax) and a hypomethylating agent (e.g. azacitidine).

### Methods

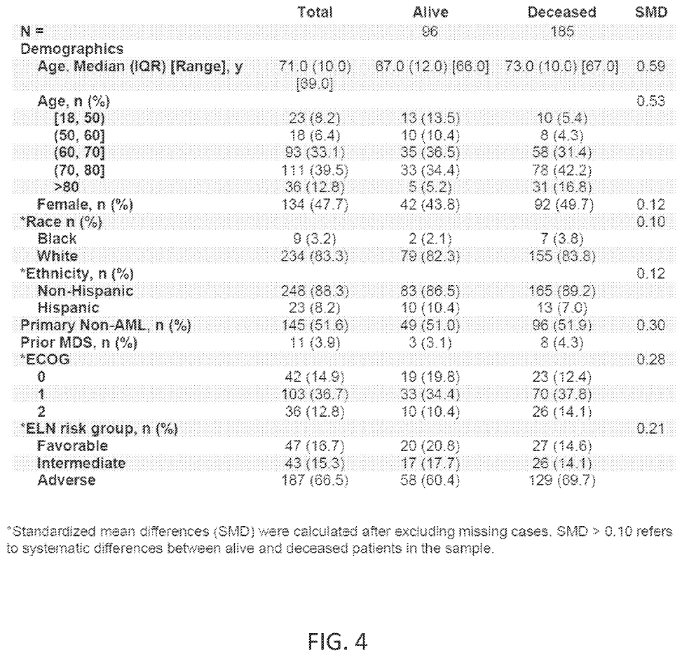

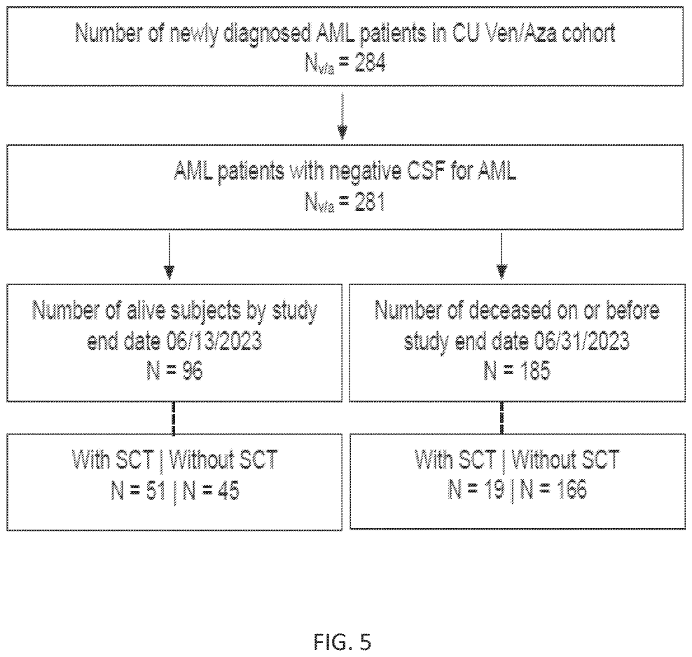

Patient populations and outcomes definitions: The patient cohort used for in the test set is summarized in FIG. 4 and details of cohort management are summarized in FIG. 5. The patient dataset included 281 real world and clinical trial treated adult patients >18 years old from the University of Colorado (CU) with newly diagnosed AML and treated with venetoclax and azacytidine (Ven/Aza) as front-line therapy. Patients were excluded who had APL and CNS involvement. Seventy patients undergoing allogeneic stem cell transplant (SCT) were censored at the time of transplant as SCT has profound effects on OS distinct from those of Ven/Aza. Important clinical parameters considered in the risk model development included patient demographics as well as diagnostic bone marrow aspirate next generation sequencing (NGS) for AML mutations, fluorescence in-situ hybridization (FISH), PCR, cytogenetic (CYT) and flow cytometric (FC) features. Complex data including NGS results and FC were simplified into binary values as described in Islam, N., et al. JCO Clin Cancer Inform 6, e2200030 (2022). Short-term treatment responses including complete remission (CR), complete remission with incomplete hematologic recovery (CRi), morphologic leukemia free state (MLFS), partial remission (PR), no response, and relapse were defined based on ELN definitions. Overall survival (OS) was defined as the time from Ven/Aza treatment initiation to all-cause mortality and event free survival (EFS) was defined as the time from Ven/Aza treatment initiation to the first documented “no response”, “partial remission”, “relapse”, and “death” from any cause.

### Statistical Analysis

Exploratory Analysis: University of Colorado patient data was structured and analyzed as described in Islam, N., et al. JCO Clin Cancer Inform 6, e2200030 (2022). Descriptive statistics were provided for baseline covariates and outcomes. Standardized mean differences (SMD) were reported along with summary statistics. Pairwise associations along with p-values (with respect to Chi-Squared test counterparts) between biomarkers were provided. Kaplan-Meier analyses were performed for time-to-event data assuming right censoring. Patients were censored at their last known reported alive date or the end of study period (i.e., Apr. 8, 2023). 95% confidence intervals (CIs) and median survival times were reported. KM survival curves were estimated based on the full analytical dataset assuming intent-to-treat (ITT), a subset excluding SCT patients, and the set treating SCT patients as censored at their corresponding transplant dates. Equality of survival curves were evaluated based on Log-rank (LR), Tarone-Ware (TW), Fleming-Harrington (FH), weighted multiple direction (mdir) LR, Max-Combo (MC), K-sample omnibus non-proportional hazard (KONP), and restricted mean survival times (RMST) tests <refs>. Corresponding p-values were reported. Univariate association filtering was used to exclude noise variables and reduce dimension. Zero-variation and low-count variables were dropped. Variables with a p-value<0.30 based on at least one univariate tests (as above) in any time-to-event responses were considered as candidates for multivariable models. The resulting combined list of covariates was retained for subsequent multivariable OS and EFS models.

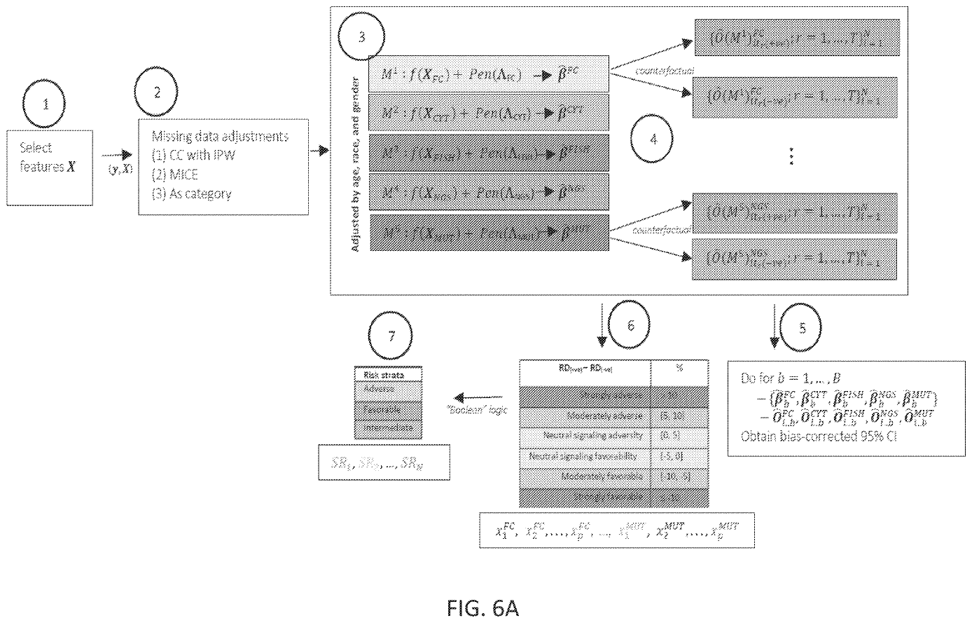

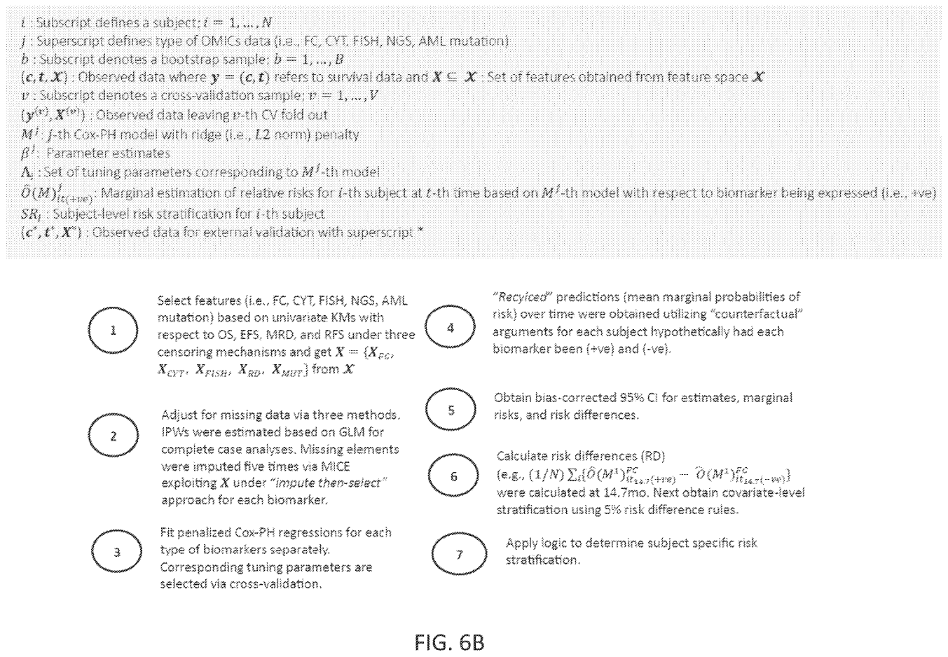

Outcome regressions and counterfactual effects estimation: The architecture of the modeling framework is illustrated in Supplementary FIGS. 6A-6B. Due to collinearity among confounders, multivariable Cox proportional hazard models with ridge penalty (mCOXr) were fitted separately on FC, NGS, FISH, AML mutations, and CYT variables. Demographic variables such as age, gender, and race were adjusted in all models to ensure that the models were not biased towards a certain group. An optimum shrinkage penalty value was selected via a 10-fold cross-validation (CV) method. 95% CIs of adjusted hazard ratios (aHRs) were constructed based on fractional random weight bootstrap (FRWB) approach of 2,000 runs. Multivariable penalized logistic regressions with ridge penalty (mLRr) were used to model CR/CRh based best response. Biomarker-specific mean predicted marginal probabilities of risk overtime were calculated via “recycled” predictions assuming counterfactual arguments with respect to the corresponding mCOXr models. At each time point, mean risks of a biomarker being positive and negative were calculated by averaging predicted marginal probabilities for all patients in the sample as if they had (hypothetically) been mutated (+ve) or wild-type (−ve) for genetic features and if they expressed a marker by FC. This approach used a fixed patient cohort for each biomarker to fairly compute marginal risks and accounted for differences in the mix and severity of risk factors among subjects. Percentile based 95% confidence intervals over time were reported exploiting estimates from 2,000 FRWB runs. The Kolmogrov-Smimov (KS) test was used to assess the differences in counterfactual risk distributions between a biomarker being positive (mutated) and negative (wild-type) (for genetic features) or expressed/not expressed for FC.

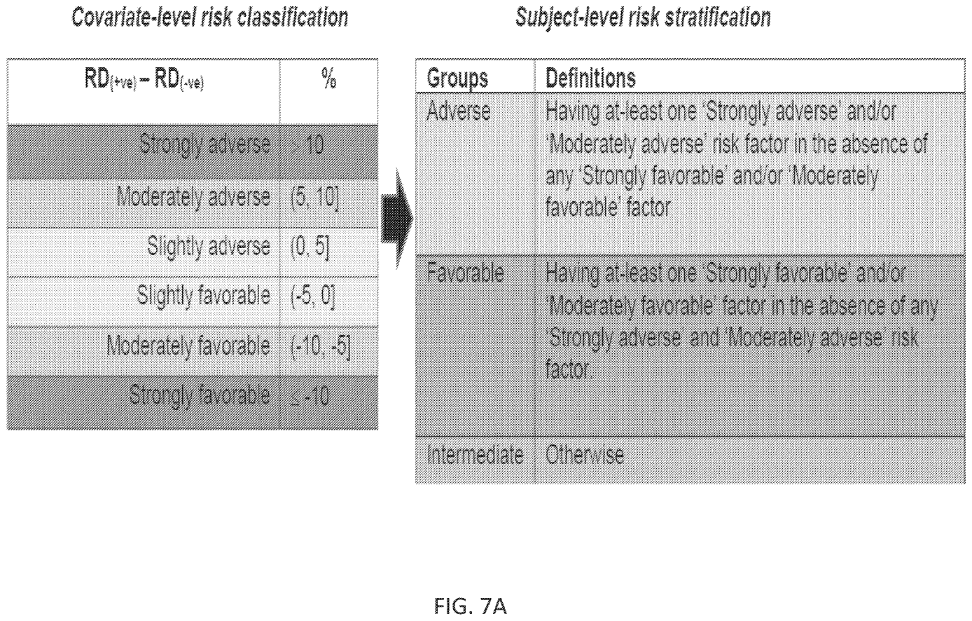

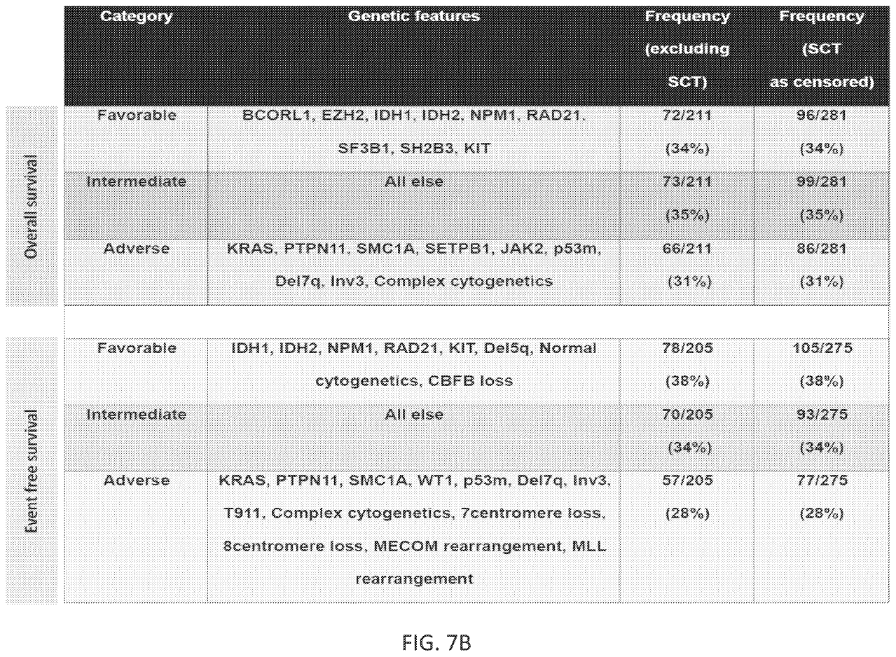

Risk stratification of baseline covariates and subjects: The overall risk model strategy is summarized in FIGS. 7A-7B Counterfactual risk differences were computed over time up to 3 years. For stratification, a specific time point such as 14.7 month was chosen based on the median OS of the VIALE-A study (see DiNardo, C. D., et al. N Engl J Med, 383, 2020, 617-629). Two level of classifications (covariate-level and subject-level) were been performed. First, the magnitude and direction of marginal risk differences (RD) between (+ve) and (−ve) at 14.7 month was used to classify each feature into “adverse” and “favorable”. Positive risk differences such as (0,5)%, (5,10)%, and >10% classified a feature into either slightly, moderately, or strongly “adverse” variable, respectively. Similarly, the negative differences categorized each into slightly, moderately, or strongly “favorable” feature. Subject-level stratification (“adverse”, “intermediate”, and “favorable”) was determined based on combining covariate-level classifications using a ‘Boolean’ (i.e., and/or) logic since a person can have multiple “favorable” and/or “adverse” features.

Evaluating generalizability. Internal validation was conducted with respect to predictability of proposed risk stratification and ELN-2022 risk group for OS and EFS. Internal validation was performed based on a 10-fold cross validation (CV). Models were trained utilizing information of 9 folds each time and computed prediction accuracy on the remaining fold. This step was repeated for each fold. Each CV run generated an independent list of risk stratification variables. Four validation strategies were adopted with respect to how risk stratification was performed—(a) keeping the original list of variables outlined in FIGS. 7A-7B, (b) classifying risk factors as in 7A-7B based on each CV run and then generating a combined list based on “maximum-votes” across 10 runs, (c) classifying risk factors as in FIGS. 7A-7B based on each CV run and then generating a combined list based on “maximum-votes” across 10 runs and over three missing data mechanisms (i.e., 30 runs), and (d) classifying risk factors as in FIGS. 7A-7B based on each training dataset. Note that (d) results in a different set of risk factors at each run and thus are not appropriate for validating predictive performance. The predictive performance was compared with respect to dynamic AUCs of cumulative case dynamic control ROC (cAUC), incident case dynamic control ROC (iAUC), and integrated Brier scores (IBS). Both the Kaplan-Meier method and model-based approach were utilized. In the former approach, a test patient was categorized into the “Adverse”, “Intermediate”, or “Favorable” group, and subject-specific survival probabilities were generated using the resulting KM estimates of the corresponding group based on the trained set. In the model-based approach, the mCOXr model was trained adjusting for age, gender, race, and either ELN-2022 or proposed risk stratification variable. Next, the performances were evaluated on test CV folds and results were concatenated.

Sensitivity analyses: To de-bias the effect of SCT on long-term survival, sensitivity analyses were performed re-running all regression models treating SCT patients as censored at their transplant dates. Primary analyses were performed using missing values as a separate category due to its simplicity as this approach does not require additional modeling to impute values or excluding missing cases. Note that the reported results (otherwise specified) were based on this approach. However, additional sensitivity analyses were performed to check robustness considering other missing data handling techniques.
(A) Running weighted models on complete cases (CC). This technique adjusted for the exclusion bias stemming from CC analysis incorporating inverse probability weights (IPW) into mCOXr and mLRr where the subject-specific weights were estimated from a separate, multivariable logistic regression model signaling the likelihood of having at-least one missing data element.
(B) Imputing missing data via multivariable regression models. This approach generated multiple imputations (5 samples) for each subject's every missing data element exploiting multivariate data sequentially (multivariate imputation by chained equations (MICE)), aHRs and aORs were averaged over 5 samples and 2,000 FRWB runs; percentile based 95% confidence intervals were reported.

Overall 15 different models were explored. Marginal contributions of genetic and phenotypic features were assessed in terms of absolute changes in median KMs of OS and EFS. Uncertainty in estimation was assessed via 2,000 FRW bootstrap based weighted KM curves.

All statistical tests, except marginal risk curves comparisons, were two-sided, with a significance level of 5%. All analyses were conducted using R, version 3.6.3.

### Results

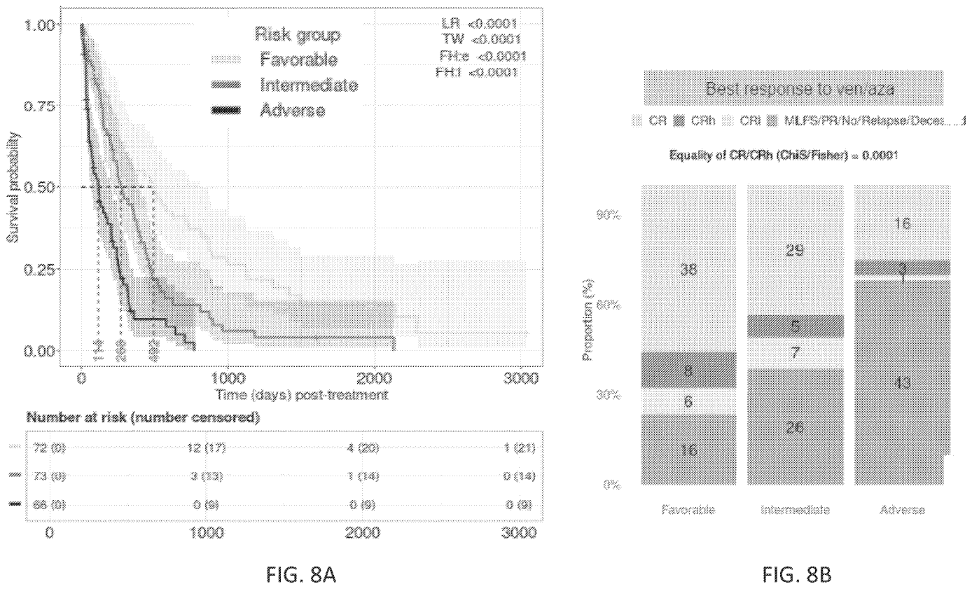

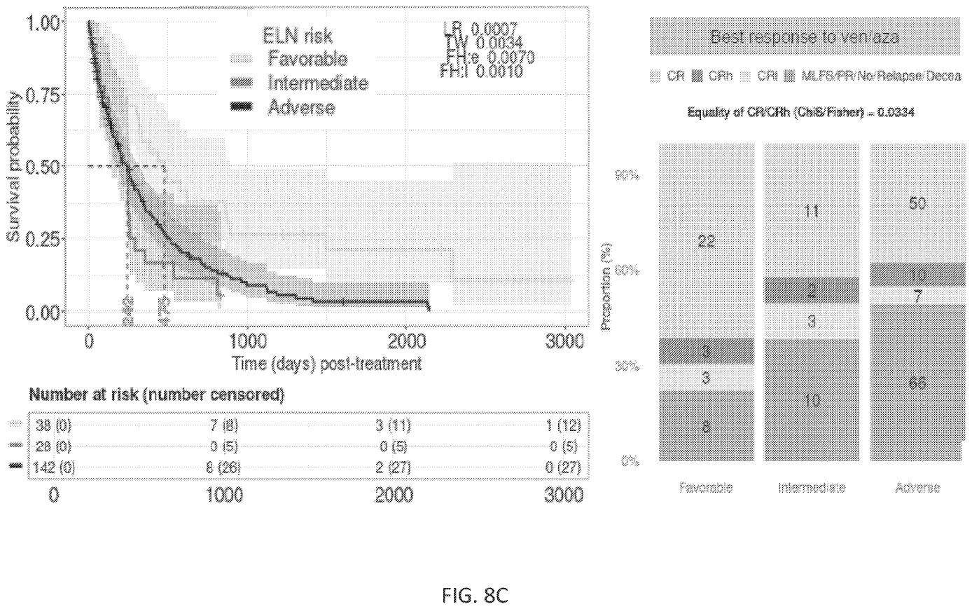

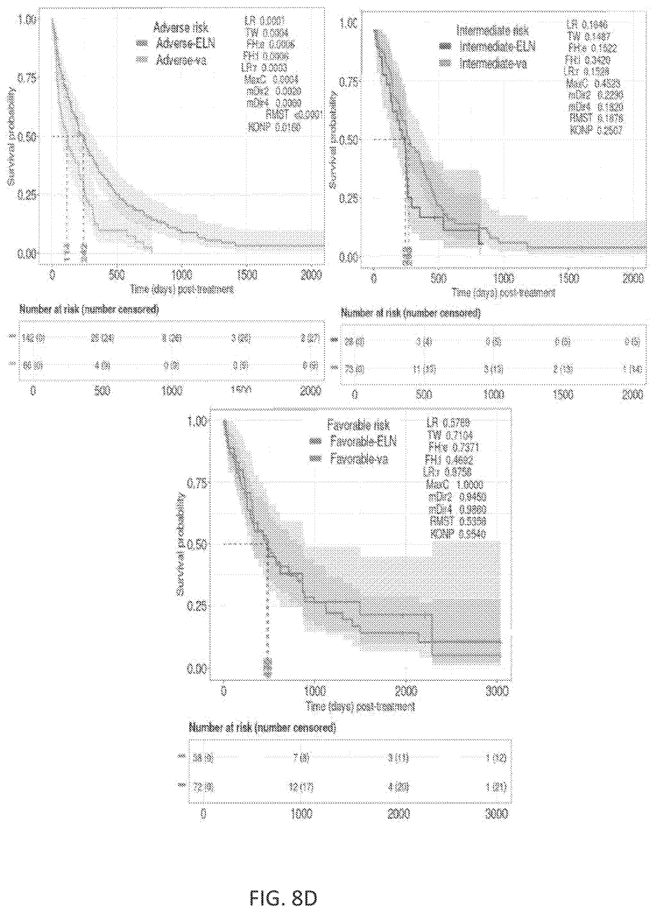

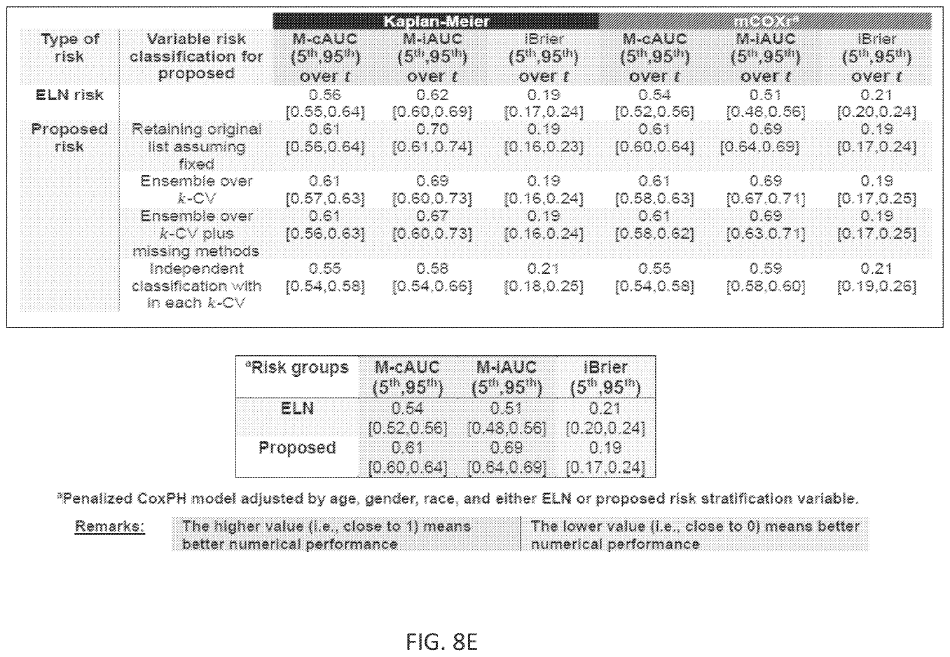

Genetic features associated with overall survival and best responses: A risk model for OS and best responses (CR, CRi, CRh and others) using commonly assessed AML genetic features was developed. A counterfactual model was used to identify individual covariates that were divided into strongly adverse, moderately adverse, slightly adverse, slightly favorable, moderately favorable and strongly favorable as described in the methods above, FIGS. 6A-6B and FIGS. 7A-7B. Subsequently, a subject specific risk classification model was created by discriminating patient populations into Favorable. Intermediate and Adverse risk groups as summarized in FIGS. 7A-7B. Important for this analysis was an analysis of 2-factor interactions between the covariates to determine confounding effects leading to the definition of subject specific risks accounting for these confounding effects. This risk model was then applied to the Ven/Aza treatment cohort with SCT patients excluded (n=211) to evaluate its association with OS for patients receiving Ven/Aza without outcomes being influenced by SCT with curative intent (FIG. 8A). The adverse risk group had a median OS of 114 days, the intermediate had a median OS of 268 days and the favorable group had a median OS of 492 days. The adverse group consisted of 66/211 patients (31%), the intermediate group 73/211 (35%) and the favorable group 72/211 patients (34%). When the same model was applied to best responses (FIG. 8B), the adverse risk group had a CR rate of 25%, intermediate 43% and favorable 56% with composite responses as defined by CR/CRh of 30%, 51% and 68% respectively (all statistically significant between risk groups). For comparison, the ELN 2022 risk criteria model was then applied to the same Ven/Aza treated cohort excluding patients undergoing SCT. These results are shown in FIG. 8C. Both the ELN 2022 adverse risk group and the intermediate group had a median OS of 242 days and the favorable group had a median OS of 475 days. The adverse group consisted of the 68% of patients, the intermediate group was 13% and the favorable group was 18%. When the same model was applied to best responses, the ELN 2022 adverse risk group had a CR rate of 38%, intermediate 42% CR and favorable 61% wCR with composite responses as defined by CR/CRh of 53%, 50% and 69% respectively. The Ven/Aza specific risk model categories were then directly compared to their corresponding ELN2022 categories. The results are shown in FIG. 8D. The adverse risk group for the Ven/Aza risk specific risk model was significantly smaller and had significantly worse outcomes relative to the ELN 2022 defined adverse risk group. The intermediate and favorable risk groups were relatively larger for the Ven/Aza specific risk model, however their OS was relatively similar with both models. Fleiss kappa analysis demonstrated a high level of discordance in patient assignment to the “intermediate” category between the two groups with the highest level of concordance found with the “favorable” risk category. Fleiss kappa for favorable, intermediate and adverse were 0.3, 0.06 and 0.20, respectively, with high values closer to 1 indicating more agreement. Additionally, AUCs and iBrier comparison for the Ven/Aza specific risk model were superior for the Ven/Aza specific risk model (FIG. 8E). Without wishing to be bound by theory, together these features confirm significant discordance between the ELN2022 and Ven/Aza specific risk models for Ven/Aza treated patients and point to the superior performance of the Ven/Aza specific model and the biomarkers identified from that model and disclosed herein.

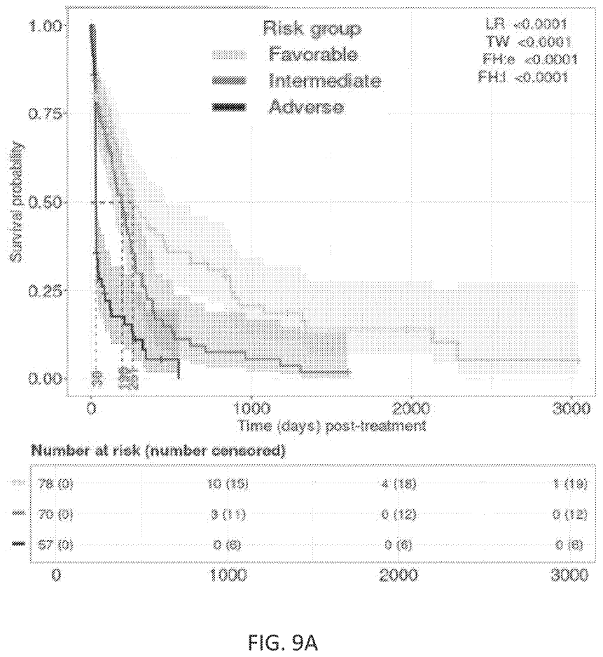

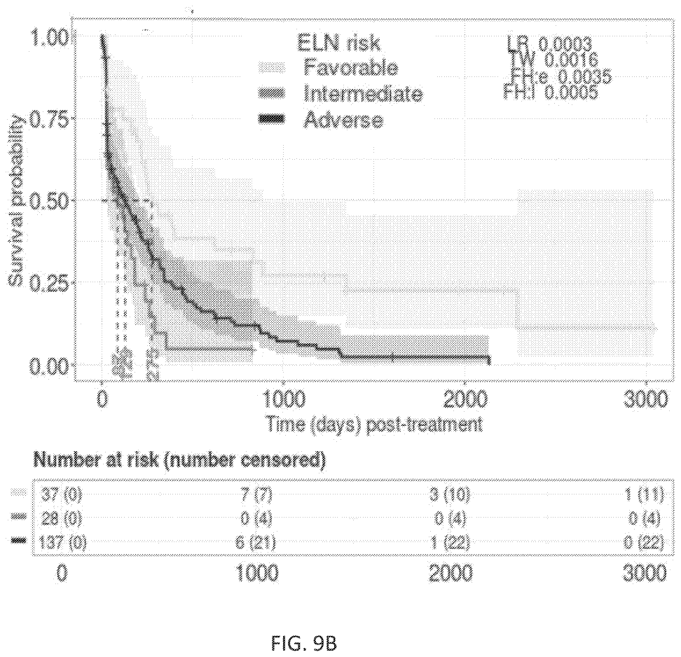

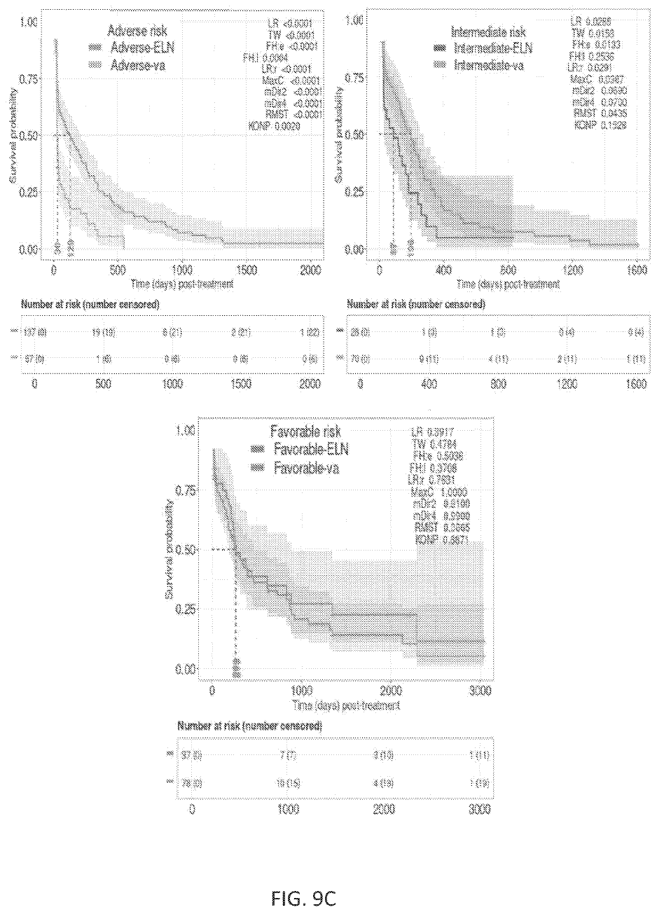

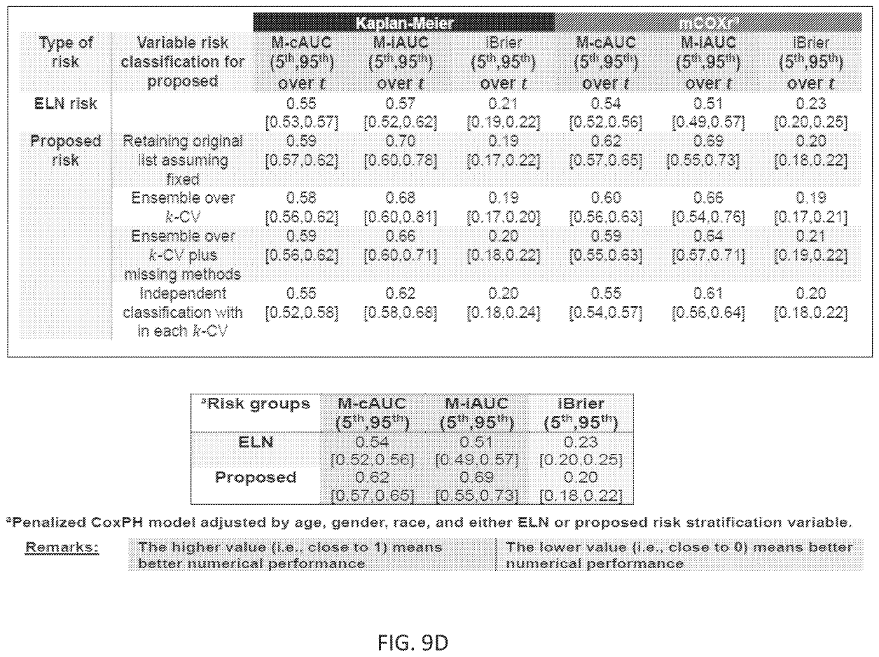

Genetic features associated with event free survival and application of the OS and EFS models to SCT patients: A EFS risk model was then defined as EFS is a critical long-term outcome in analysis of AML treatment effects. The same methodologies were used as described for the OS model, and while there were overlaps in features of the OS and EFS models, specific adverse and favorable risk factors were identified that differed between the models. These included absence of BCORL1, SFB1, and SH2B3 and presence of Del 5q, normal cytogenetics and loss of CBFB in the EFS favorable risk category relative to the favorable risk OS category. For the adverse risk group SETPB1 and jak2 were absent and t9:11, 7 centromere loss, 8 centromere loss, MECOM rearrangement and MLL rearrangement were all associated with adverse EFS. This Ven/Aza treatment EFS specific model effectively distinguished adverse, intermediate and favorable risk groups with median EFS of 30, 196 and 261 days respectively (FIG. 9A). These subgroups comprised 28%, 34% and 38% of the entire cohort respectively. For comparison, the ELN 2022 risk criteria model was then applied to the same Ven/Aza treated cohort as above for OS (FIG. 9B). Both the ELN 2022 adverse risk group had a median EFS of 129 days, the intermediate group had a lower median EFS of 87 days and the favorable group had a median EFS of 275 days. The adverse group consisted of the 68% of patients, the intermediate group was 14% and the favorable group was 18%. When the Ven/Aza specific risk model categories were then directly compared to their corresponding ELN2022 categories (FIG. 9C), the adverse Ven/Aza specific risk model had significantly worse EFS than its ELN 2022 counterpart. The Ven/Aza specific EFS intermediate risk group had a better EFS than its ELN counterpart while the favorable risk groups were similar (FIG. 9C). Fleiss kappa analysis demonstrated relatively low level of concordance of patient assignment across all three subgroups. Fleiss kappa for favorable, intermediate and adverse were 0.17, −0.236 and 0.12, respectively, with high values closer to 1 indicating more agreement. Again, the AUCs and iBrier comparison favored the Ven/Aza specific EFS risk model (FIG. 9D). Without wishing to be bound by theory, together these features confirm significant discordance between the ELN2022 and Ven/Aza specific risk models for Ven/Aza treated patients and point to the superior performance of the Ven/Aza specific model and the biomarkers identified from that model and disclosed herein.

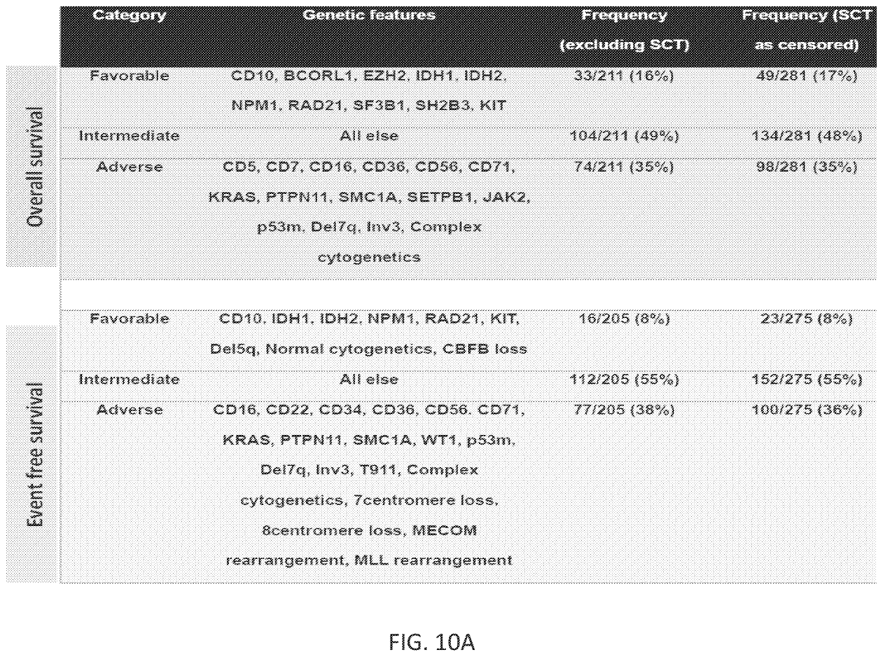

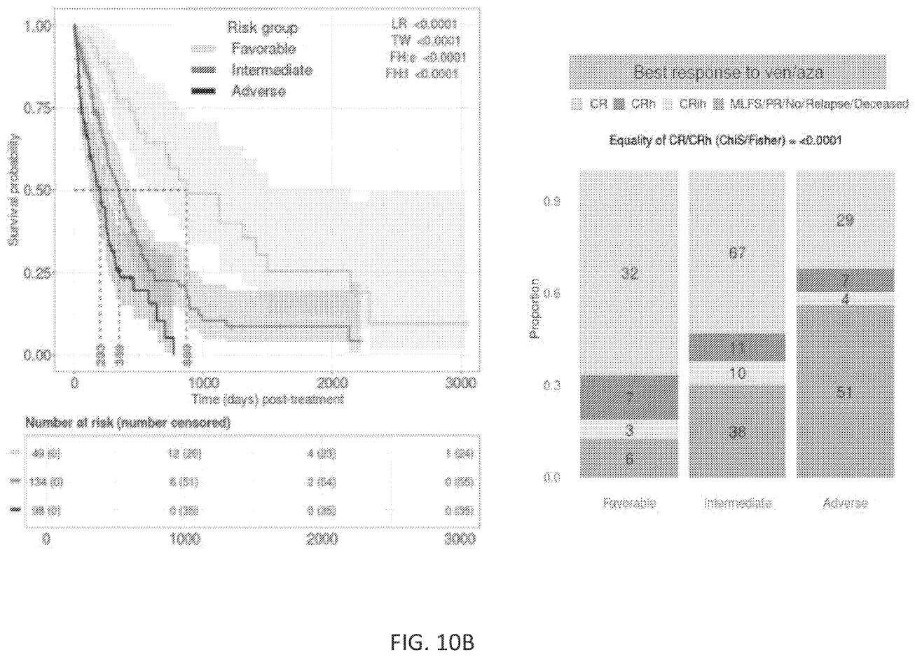

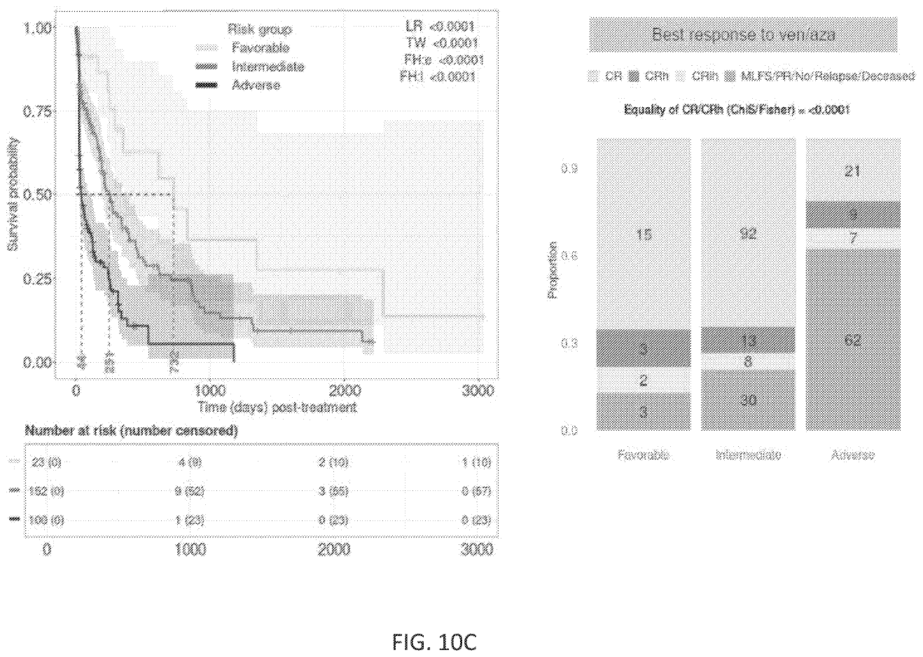

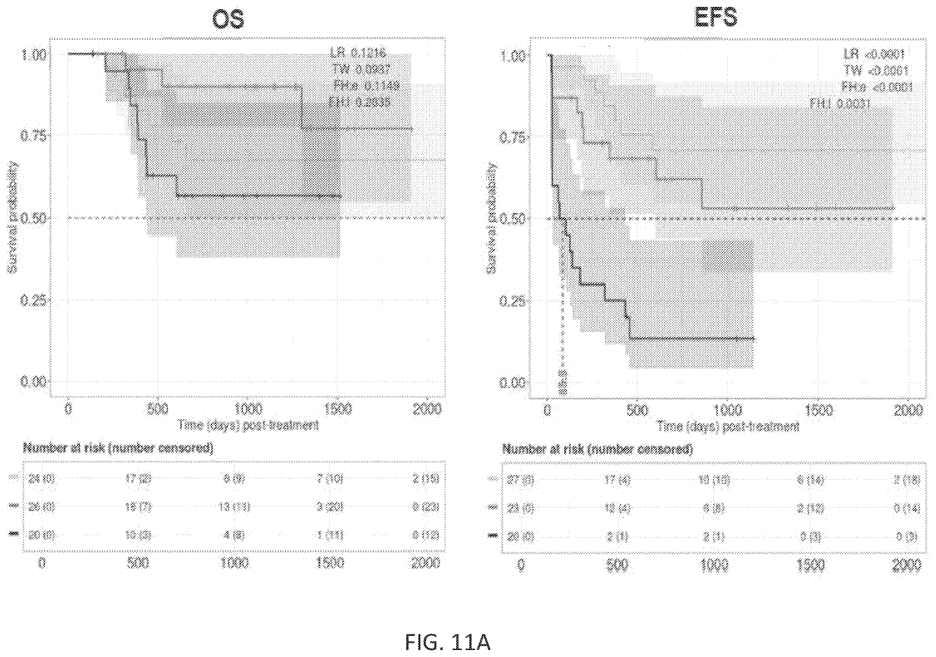

Further model refinements: Next further refinement of the OS model using additional AML features was explored. Since flow cytometry is widely used in the diagnostic evaluation of AML, we combined genetic and flow cytometric (phenotypic) data and performed the same type of risk model development for OS, best response and EFS as described above for genetic features alone. For the OS model CD10 expression on the AML was identified as a favorable feature while CD5, CD7, CD16m CD36m CD56 and CD71 were identified as unfavorable features (FIG. 10A). For the EFS risk model, similar phenotypic features were identified with the addition of CD22, and CD34 as adverse features (FIG. 10A). As with the genetic feature only model, the combined genetic plus phenotypic feature model effectively distinguished adverse, intermediate and favorable risk groups for OS, best response and EFS. Of note, the favorable risk group defined by genetic plus phenotypic features had a median OS of 869 days, thus identifying a relatively very favorable subgroup compared to any others we have identified to date. Similarly the CR rate of this very favorable subgroup was significantly better than the intermediate and adverse subgroups with a best response CR rate of 67% and a composite CR/CRh of 81% (FIG. 10B). The genetic plus phenotypic EFS model similarly yielded a significant discrimination between adverse, intermediate and favorable risk groups (FIG. 0C). Since the OS and EFS Ven/Aza specific models appeared strong for up front Ven/Aza treated patients we next tested whether they might also be applicable to OS and EFS for the relatively small subset of Ven/Aza treated patients who were ultimately able to undergo SCT (FIGS. 11A and 11B). The genetic and genetic plus risk factor models did not discriminate OS or EFS outcomes between the favorable and intermediate risk groups for either. However, the adverse risk group did relatively poorly compared to the others particularly for EFS where both models predicted EFS outcomes of <25%. Without wishing to be bound by theory, this demonstrates that the adverse risk features were sufficiently potent that they may have overcome attempts at curative therapy for the small group of Ven/Aza patients who sufficiently recovered following initial therapy to become SCT candidates.

### Summary of Example 2

In this example, a series of AML genetic features were identified that effectively predict adverse, intermediate and relatively favorable OS, best responses and EFS in newly diagnosed elderly/unfit patients treated front line with venetoclax and azacytidine. The model was specifically created using SCT censored data and applied it to SCT excluded cohort to keep effects of Ven/Aza outcomes as clear as possible without impact by SCT in both the model generation as well as model testing and validation as a) SCT is potentially curative thus profoundly impacting survival analysis and b) only small proportions of elderly/unfit patients ultimately undergo transplant. This new Ven/Aza specific risk model was generated using NGS and cytogenetics features from diagnostic marrows and was superior to the existing ELN 2022 criteria in distinguishing short- and long-term responses specifically in Ven/Aza treated elderly/unfit AML patients.

Adverse AML features in the Ven/Aza specific OS model include KRAS, PTPN11, SMC1A, SETPB1, Jak 2, p53 mutations by NGS, and Del7q, Inv3 and complex cytogenetics. In contrast favorable features include BCORL1. EZH2, NPM1, IDH1/IDH2, RAD21, SF3B11, SH2B3 and kit mutations by NGS. Several of these features vary from the ELN 2022 criteria and other risk models which is not surprising given that the ELN criteria are primarily based on patients receiving intensive induction chemotherapy, with a high proportion of patients undergoing SCT included in the risk stratification model, some features such as TP53 mutation appear to be poor prognostic features regardless of treatment and some including IDHs, and NPM1 are good prognostic features shared between the different models. Of note, the median OS (~4 months) and best responses (CR ~16%) of the adverse risk group remains strikingly low despite the improvement for all elderly/unfit AML with the advent of venetoclax/HMA therapy. This highlights an urgent ongoing need for new and improved therapies for this subgroup of AML patients. Additionally, the median OS of ~9 months in the intermediate risk group and ~16 months in the relatively favorable risk group also remains disappointing and so for all patients other than those that can ultimately undergo SCT, developing new therapies for all patients with elderly/unfit AML remains an urgent medical problem. In summary, this example describes the development of a model for assigning risks specifically for elderly/unfit AML patients treated with Ven/Aza. Accordingly, the biomarkers described herein identified in the model can be used to predict the response of a patient to treatment with a combination of a BCL-2 inhibitor and an HMA.

### Example 3

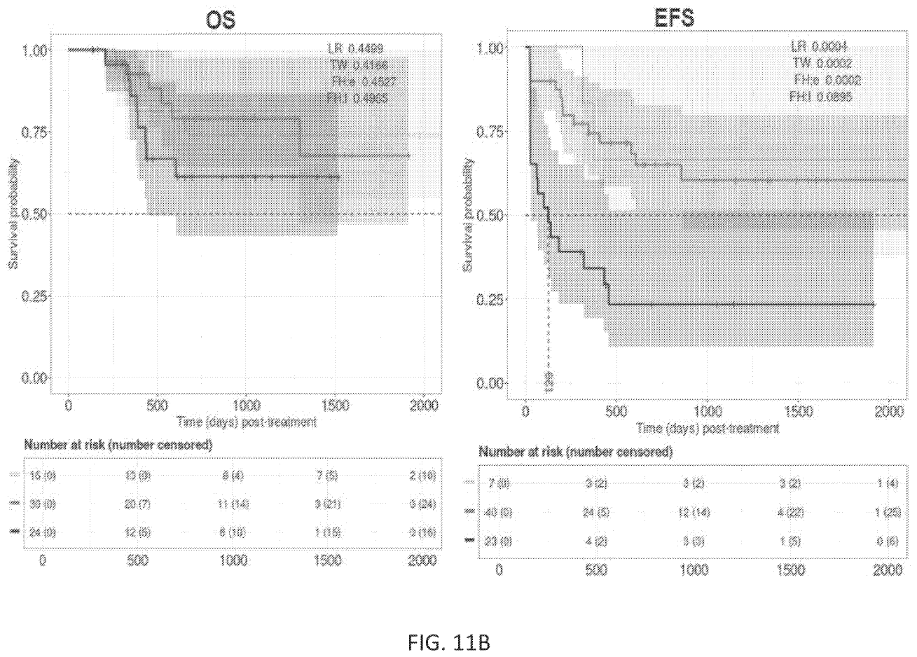

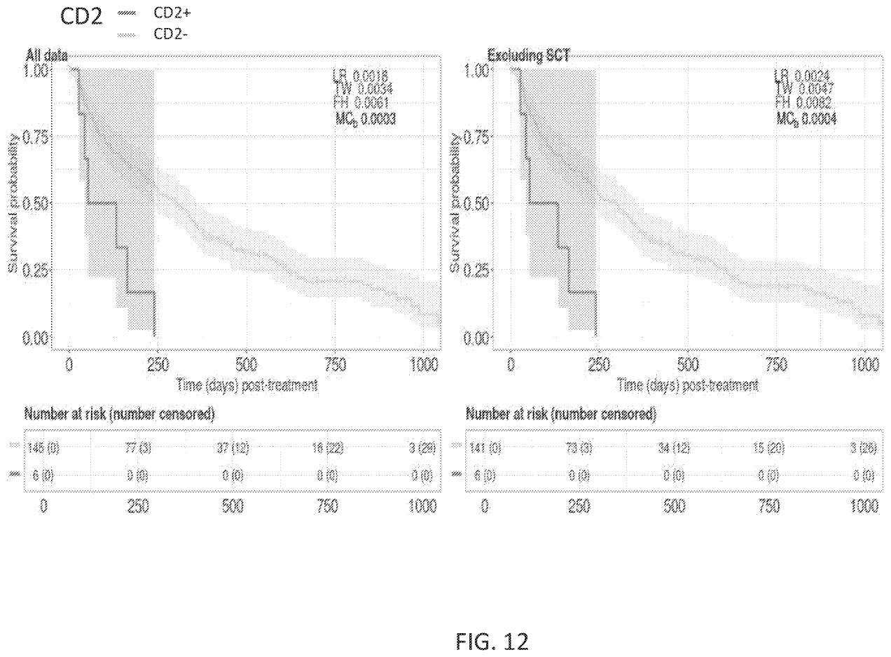

The following is a non-limiting example of analysis showing the response of AML subjects to treatment with a combination of venetoclax and azacitidine, or a combination of venetoclax, azacitidine and cusatuzumab, based on various different biomarkers. The results of this analysis are shown in FIGS. 12-18.

FIGS. 12-15 are a series of graphs showing the survival probability of AML subjects after receiving a combination of venetoclax, azacitidine and cusatuzumab. The left panel of each of FIGS. 12-15 are all subjects for which data was available, and the right panel is a subset of the data presented in the left panel minus the subjects who received at least one stem cell transplant.

FIG. 12 shows that subjects who exhibit a CD2− AML phenotype benefited more from treatment with the combination of venetoclax, azacitidine and cusatuzumab as opposed to subjects having a CD2+ AML phenotype.

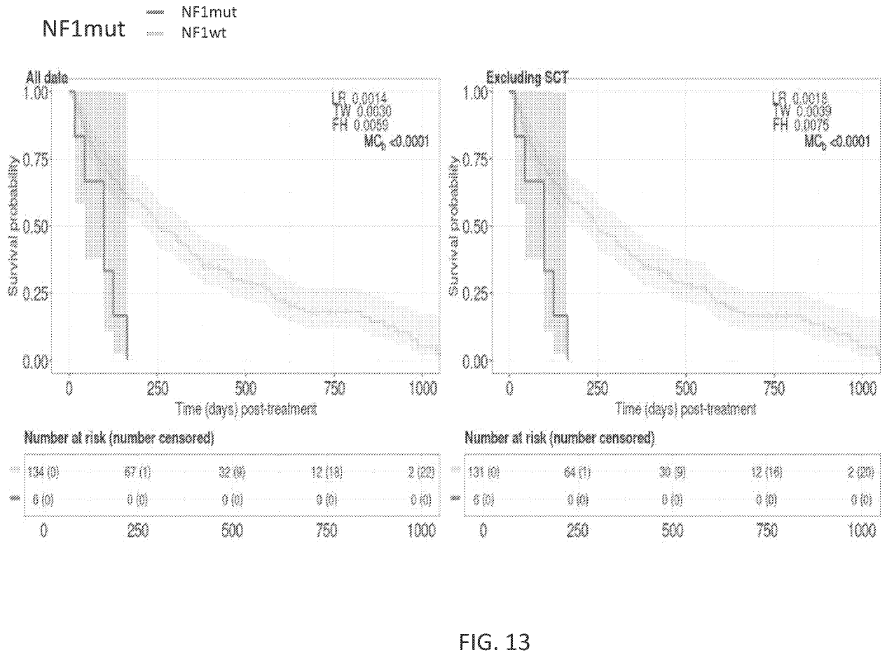

FIG. 13 shows that subjects who exhibit a wildtype NF1 benefited more from treatment with the combination of venetoclax, azacitidine and cusatuzumab as opposed to subjects having a mutated NFL.

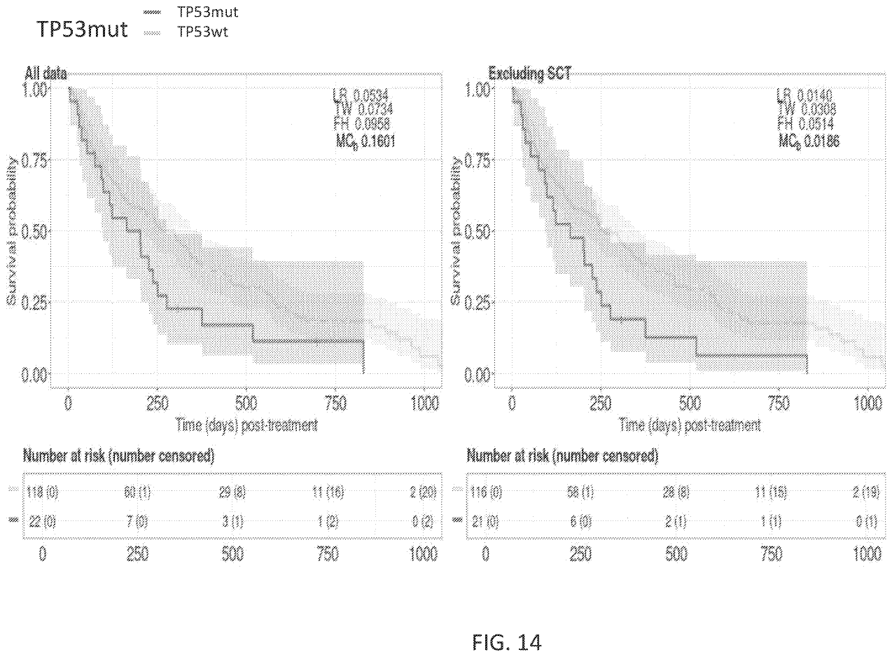

FIG. 14 shows that subjects who exhibit a wildtype TP53 benefited more from treatment with the combination of venetoclax, azacitidine and cusatuzumab as opposed to subjects having a mutated TP53.

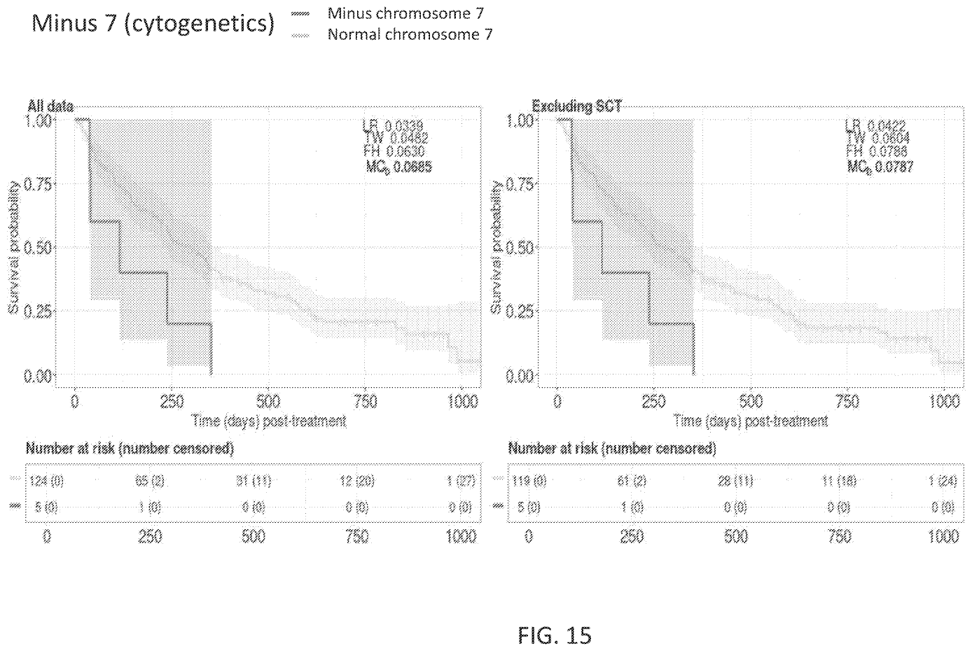

FIG. 15 shows that subjects who exhibit a normal chromosome 7 phenotype benefited more from treatment with the combination of venetoclax, azacitidine and cusatuzumab as opposed to subjects having a chromosome 7 deletion phenotype.

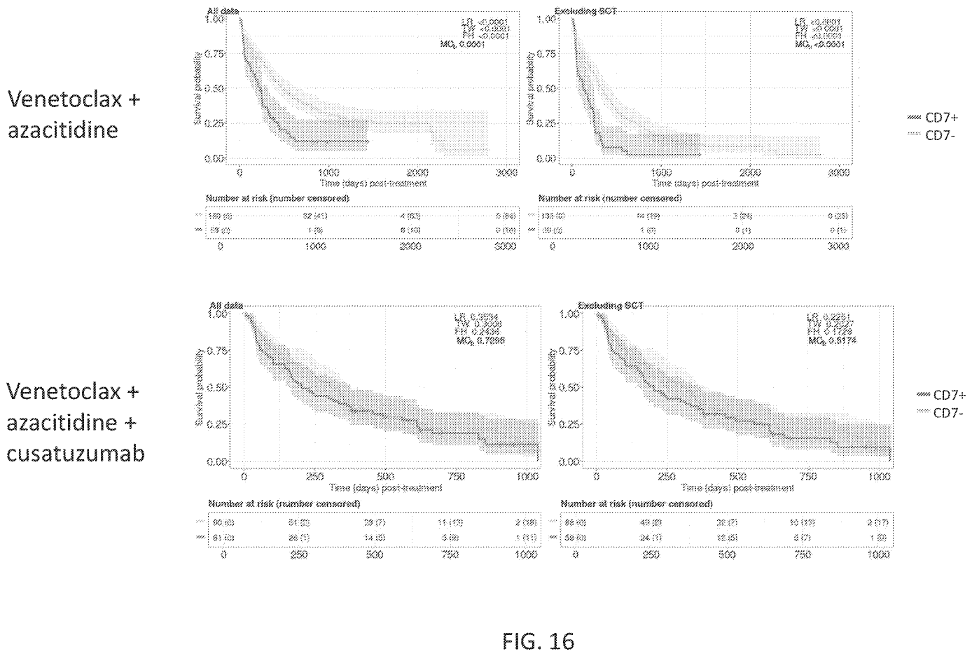

FIGS. 16-18 are a series of graphs showing the survival probability of AML subjects after receiving either a combination of venetoclax and azacitidine (top panels) or a combination of venetoclax, azacitidine and cusatuzumab (bottom panels).

FIG. 16 shows that subjects with a CD7+ AML phenotype did not benefit as much from treatment with venetoclax and azacitidine as compared to subjects with a CD7− phenotype. FIG. 5 also shows that the addition of cusatuzumab can be used to overcome the poor prognostic significant of CD7+ AML.

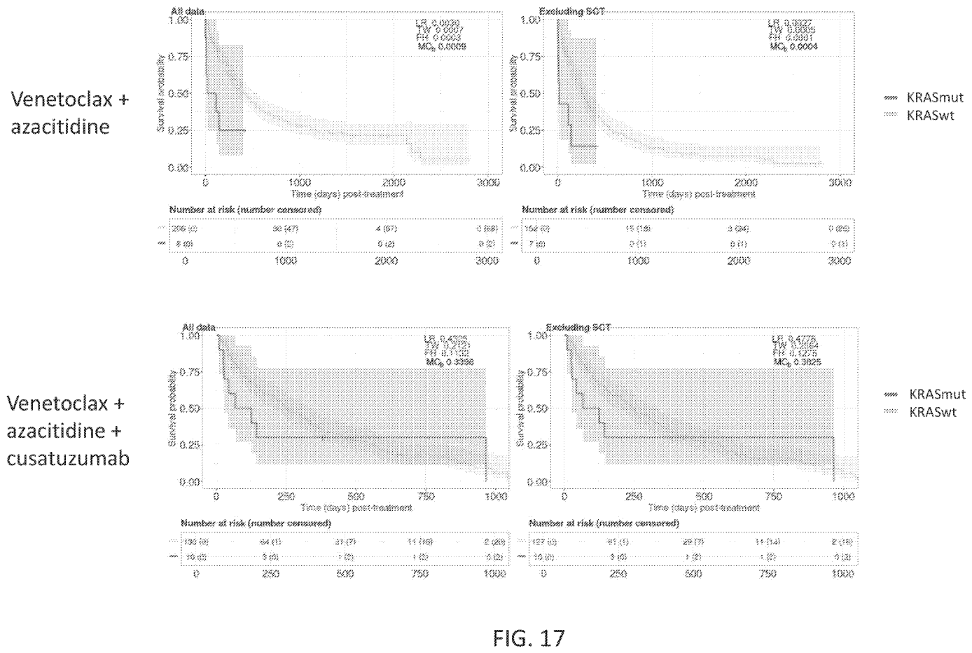

FIG. 17 shows that subjects with mutated KRAS did not benefit as much from treatment with venetoclax and azacitidine as compared to subjects with wildtype KRAS. FIG. 6 also shows that the addition of cusatuzumab can be used to overcome the poor prognostic significant of mutated KRAS.

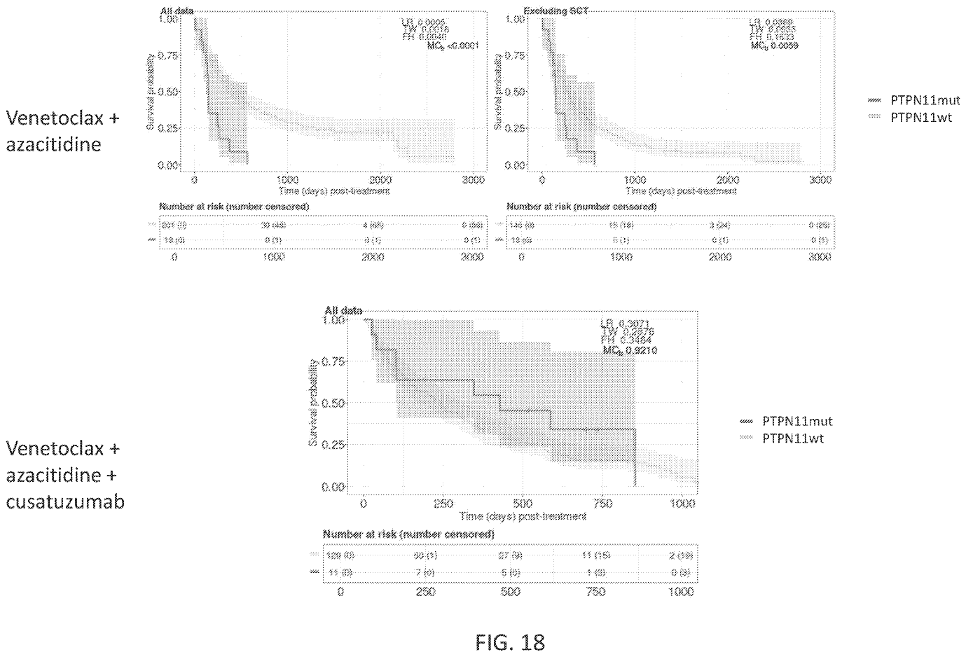

FIG. 18 shows that subjects with mutated PTPN11 did not benefit as much from treatment with venetoclax and azacitidine as compared to subjects with wildtype PTPN11. FIG. 7 also shows that the addition of cusatuzumab can be used to overcome the poor prognostic significant of mutated KRAS.

1. A method of identifying that a subject having AML will be responsive to an AML treatment comprising the administration of a combination of at least one BCL-2 inhibitor and at least one hypomethylating agent, wherein:
(i) the method comprises:
a) determining the mutational status of at least one of BCORL1, EZH2, IDH1, IDH2, NPM1, RAD21, SF3B1, SH2B3 and KIT in a plurality of cells in a sample from the subject; and
b) identifying that the subject will be responsive to the AML treatment when the presence of at least one mutation in at least one of BCORL1, EZH2, IDH1, IDH2, NPM1, RAD21, SF3B1, SH2B3 and KIT is identified,
(ii) the method comprises:
a) determining the cytogenetic status of a plurality of cells in a sample from the subject; and
b) identifying that the subject will be responsive to the AML treatment when the presence of at least cell exhibiting at least one of:
i) a Del5q phenotype;
ii) a CBFB Loss phenotype; and
iii) a Normal Cytogenetics phenotype
is identified; and/or
(iii) the method comprises:
a) measuring the expression of at least CD10 in a plurality of cells in a sample from the subject;
b) identifying the presence of at least one CD10+ cell based on the expression measured in step (a); and
c) identifying that the subject will be responsive to the AML treatment when the presence of at CD10+ cell is identified.

2-3. (canceled)

4. The method of claim 1, further comprising administering to the subject identified as being responsive to the AML treatment at least one amount of at least one BCL-2 inhibitor and at least one amount of at least one hypomethylating agent.

5-10. (canceled)

11. The method of claim 1, wherein the at least one hypomethylating agent is selected from azacitidine and decitabine.

12. The method of claim 1, wherein the at least one BCL-2 inhibitor is selected from venetoclax and navitoclax.

13. The method of claim 1, wherein step (a) comprises performing cytogenic analysis, routine karyotyping, fluorescent in situ hybridization (FISH), comparative genomic hybridization (CGH), array comparative genomic hybridization (aCGH), PCR, high-throughput sequencing, next generation sequencing (NGS), Northern Blot, reverse transcription PCR (RT-PCR), real-time PCR (qPCR), quantitative PCR, qRT-PCR, flow cytometry, mass spectrometry, microarray analysis, digital droplet PCR, Western Blot, Cellular Indexing of Transcriptomes and Epitopes by Sequencing (CITE-SEQ), any single-cell omics technology or any combination thereof.

14. The method of claim 1, wherein the subject is:
i) a subject having AML who has not received any treatment for AML; or
ii) a subject having AML who has received previously received at least one AML treatment, preferably wherein the at least one AML treatment comprises a combination of at least one BCL-2 inhibitor and at least one hypomethylating agent.

15. The method of claim 1, wherein the subject is:
i) a subject that has not received a stem cell transplant; or
ii) a subject that has previously received at least one stem cell transplant.

16. The method of claim 1, wherein:
identifying that a subject will be responsive to the AML treatment comprises identifying that the subject will have a durable remission after receiving the AML treatment; and/or
identifying that a subject will not be responsive to the AML treatment comprises identifying that the subject will be refractory to the AML treatment and/or that the subject will suffer a relapse after receiving the AML treatment.

17. (canceled)

18. The method of claim 1, wherein the biological sample comprises blood, a bone marrow biopsy, a bone marrow aspirate, a biopsy of a chloroma, a tissue biopsy, cerebrospinal fluid or any combination thereof.

19. The method of claim 1, wherein the plurality of cells comprises acute myeloid leukemia cells, optionally wherein the acute myeloid leukemia cells comprise acute myeloid leukemia blast cells, leukemia stem cells, or any combination thereof.

20. (canceled)

21. A method of identifying that a subject having AML will not be responsive to an AML treatment comprising the administration of a combination of at least one BCL-2 inhibitor and at least one hypomethylating agent, wherein:
(i) the method comprises:
a) determining the mutational status of at least one of KRAS, PTPN11, SMC1A, WT1, SETPB1, JAK2, and p53 in a plurality of cells in a sample from the subject;
b) identifying that the subject will not be responsive to the AML treatment when the presence of at least one mutation in at least one of KRAS, PTPN11, SMC1A, WT1, SETPB1, JAK2, and p53 is identified,
(ii) the method comprises:
a) measuring the expression of at least one of CD5, CD7, CD16, CD22, CD34, CD36, CD56, and CD71 in a plurality of cells in a sample from the subject;
b) identifying the presence of at least one cell that expresses at least one of CD5, CD7, CD16, CD22, CD34, CD36, CD56, and CD71 based on the expression measured in step (a);
c) identifying that the subject will not be responsive to the AML treatment when the presence of at cell expressing at least one of CD5, CD7, CD16, CD22, CD34, CD36, CD56, and CD71 is identified, and/or
(iii) the method comprises:
a) determining the cytogenetic status of a plurality of cells in a sample from the subject; and
b) identifying that the subject will not be responsive to the AML treatment when the presence of at least cell exhibiting at least one of:
i) a Del7q phenotype;
ii) a Inv3 phenotype;
iii) a Complex Cytogenetics phenotype;
iv) a translocation t(9; 11) phenotype;
v) a 7 centromere loss phenotype;
vi) an 8 centromere loss phenotype;
vii) a MECOM rearrangement phenotype; and
viii) a MLL rearrangement phenotype
is identified.

22. The method of claim 21, the method further comprising administering to the subject identified as not being responsive to AML treatment at least one alternative AML treatment, wherein the at least one alternative AML treatment comprises: i) does not comprise a BCL-2 inhibitor in combination with an HMA; or ii) comprises the administration of a combination of at least one BCL-2 inhibitor, at least one HMA, and at least one additional agent.

23. The method of claim 22, wherein the at least one additional agent is a CD70-targeting agent.

24. The method of claim 23, wherein the at least one CD70-targeting agent comprises:
i) an anti-CD70 antibody, preferably wherein the anti-CD70 antibody is cusatuzumab;
ii) an anti-CD70 immunotherapy, preferably wherein the immunotherapy comprises CAR-T and/or NK Cells that are directed specifically at CD70;
iii) an agent that blocks CD70 signaling, preferably wherein the agent that blocks CD70 signaling prevents binding of CD27 and CD70.

25. The method of claim 21, wherein step (a) comprises performing cytogenic analysis, routine karyotyping, fluorescent in situ hybridization (FISH), comparative genomic hybridization (CGH), array comparative genomic hybridization (aCGH), PCR, high-throughput sequencing, next generation sequencing (NGS), Northern Blot, reverse transcription PCR (RT-PCR), real-time PCR (qPCR), quantitative PCR, qRT-PCR, flow cytometry, mass spectrometry, microarray analysis, digital droplet PCR, Western Blot, Cellular Indexing of Transcriptomes and Epitopes by Sequencing (CITE-SEQ), any single-cell omics technology or any combination thereof.

26. The method of claim 21, wherein the subject is:
i) a subject having AML who has not received any treatment for AML; or
ii) a subject having AML who has received previously received at least one AML treatment, preferably wherein the at least one AML treatment comprises a combination of at least one BCL-2 inhibitor and at least one hypomethylating agent.

27. The method of claim 21, wherein the subject is:
i) a subject that has not received a stem cell transplant; or
ii) a subject that has previously received at least one stem cell transplant.

28. The method of claim 21, wherein:
identifying that a subject will be responsive to the AML treatment comprises identifying that the subject will have a durable remission after receiving the AML treatment; and/or
identifying that a subject will not be responsive to the AML treatment comprises identifying that the subject will be refractory to the AML treatment and/or that the subject will suffer a relapse after receiving the AML treatment.

29. The method of claim 21, wherein the biological sample comprises blood, a bone marrow biopsy, a bone marrow aspirate, a biopsy of a chloroma, a tissue biopsy, cerebrospinal fluid or any combination thereof.

30. The method of claim 21, wherein the plurality of cells comprises acute myeloid leukemia cells, optionally wherein the acute myeloid leukemia cells comprise acute myeloid leukemia blast cells, leukemia stem cells, or any combination thereof.
